(19) United States
(12) Patent Application Publication
(10) Pub. No.: US 2018/0104332 A1
(43) Pub. Date: Apr. 19, 2018

(54) INSULIN-LIKE GROWTH FACTOR INHIBITOR AND CHEMOTHERAPEUTIC AGENT FOR USE IN CANCER THERAPY

(71) Applicant: The University of Liverpool, Liverpool (GB)

(72) Inventors: Ainhoa Mielgo Iza, Liverpool (GB); Michael Schmid, Liverpool (GB)

(21) Appl. No.: 15/543,736

(22) PCT Filed: Jan. 15, 2016
(86) PCT No.: PCT/GB2016/050092

§ 371 Date: Jul. 14, 2017

(30) Foreign Application Priority Data
Jan. 16, 2015 (GB) 1500760.2
Feb. 26, 2015 (GB) 1503273.3

(51) Int. Cl. A61K 39/395; A61K 31/7068; A61P 35/00; C07K 16/22; A61K 31/337

(52) U.S. Cl. CPC A61K 39/3955; A61K 31/7068; A61K 2039/505; C07K 16/22; A61K 31/337; A61P 35/00

(57) ABSTRACT

The present invention relates to combinations and pharmaceutical compositions comprising an IGF inhibitor and a chemotherapeutic agent, and their use in treating a proliferative disorder such as cancer (for example a solid cancer such as pancreatic cancer). The invention also provides an IGF inhibitor for use in treating such proliferative disorders in combination with a chemotherapeutic agent, and a chemotherapeutic agent for use in treating such proliferative disorders in combination with an IGF inhibitor and a biomarker for identifying proliferative disorders which have increased responsiveness to the combined treatment.

The present invention relates to combinations and pharmaceutical compositions comprising an IGF inhibitor and a chemotherapeutic agent, and their use in treating a proliferative disorder such as cancer (for example a solid cancer such as pancreatic cancer). The invention also provides an IGF inhibitor for use in treating such proliferative disorders in combination with a chemotherapeutic agent, and a chemotherapeutic agent for use in treating such proliferative disorders in combination with an IGF inhibitor and a biomarker for identifying proliferative disorders which have increased responsiveness to the combined treatment.

### BACKGROUND

Proliferative disorders such as cancer pose a significant health problem worldwide. Current options for treating such disorders include surgical resection, external beam radiation therapy and/or systemic chemotherapy. Such treatments are partially successful for some forms of cancer but are less successful for others. Drug resistance is one of the biggest challenges in cancer therapeutics and is the cause of relapse in the majority of cancer patients, including pancreatic cancer patients (Zahreddine and Borden, 2013).

By way of example, gemcitabine is the standard chemotherapeutic treatment for pancreatic cancer (also known as pancreatic ductal adenocarcinoma (PDA)) and yet 95% of patients diagnosed with pancreatic cancer and treated with gemcitabine die within 5 years of commencing treatment. The low efficacy of gemcitabine in the treatment of PDA is in part due to the ability of pancreatic cancer cells to become resistant to gemcitabine.

Understanding the molecular mechanisms underlying the development of cancer cell resistance to chemotherapy is critical to the development of durable treatment strategies for such proliferative disorders.

Although multiple factors can contribute to the resistance of cancers such as PDA to chemotherapeutic therapies, one dominant player is the presence of a rich pro-tumoral microenvironment (Gore and Korc, 2014; Lunardi et al., 2014; McMillin et al., 2013; Mielgo and Schmid, 2013; Noy and Pollard, 2014). Tumour associated macrophages (TAMs) are key drivers of this pro-tumoral microenvironment and can promote tumour cell proliferation, drug resistance and metastasis in many cancers including PDA (Noy and Pollard, 2014). Thus, high numbers of TAMs often correlate with resistance to chemotherapy and metastasis leading to poor survival in pancreatic cancer patients (Ino et al., 2013; Kurahara et al., 2011). Previous studies have shown that inhibition of myeloid cell infiltration into the tumour restrains cancer progression (Schmid et al., 2011a; Schmid et al., 2013). However, TAMs can be polarised into M1 inflammatory/anti-tumoral macrophages that will activate an immune response against the tumour or M2 immunosuppressive/pro-tumoral macrophages that promote tumour immunity (Kurahara et al., 2011; Mielgo and Schmid, 2013; Ruffell et al., 2012).

TAMs are present in the tumour microenvironment of many cancers, especially solid cancers such as pancreatic cancer, lung cancer, breast cancer, melanoma, colorectal cancer, ovarian cancer, gastric cancer, thyroid cancer, liver cancer and prostate cancer. Therapeutics that specifically inhibit the pro-tumoral functions of TAMs, while sparing their anti-tumoral capacity, hold great promise in the goal of restraining cancer progression, metastasis and relapse.

There is a clear need for new therapeutic agents and methods for the treatment of proliferative disorders such as cancer.

### BRIEF SUMMARY OF THE DISCLOSURE

The inventors have investigated the mechanisms by which pro-tumoral TAMs impact PDA and breast cancer resistance to chemotherapy and tumour progression.

The invention is based on the surprising finding that M2-like macrophages express high levels of IGFs, such as IGF-1 and IGF-2 and are an important source of these growth factors in the tumour microenvironment. Surprisingly, the inventors have identified that secretion of IGF-1 and IGF-2 by M2-like macrophages leads to the activation of Insulin Receptor signaling and subsequent cancer cell resistance to chemotherapy. The inventors have also found that within the tumour microenvironment, M2-like macrophages and αSMA+ stromal cells are the main sources of IGFs.

Advantageously, the inventors have found that directly blocking IGF binding, as opposed to blocking the IGF receptors, counteracts this resistance to chemotherapy and restores sensitivity to chemotherapeutic agents, resulting in reduced tumour growth in pancreatic cancer mice treated with a combination of chemotherapeutic agent (gemcitabine) and an IGF inhibitor (an IGF-blocking antibody). Furthermore, the inventors have shown that directly blocking IGF binding also counteracts pancreatic cancer cell resistance to paclitaxel and 5′FU (also known as fluorouracil herein), two additional chemotherapeutic agents commonly used in the treatment of pancreatic cancer.

These novel findings indicate that combinations and pharmaceutical compositions comprising an IGF inhibitor (e.g. an IGF blocking antibody) and a chemotherapeutic agent (e.g. gemcitabine and/or 5′FU and/or nab-paclitaxel) are useful in the treatment of a proliferative disorder such as cancer (for example a solid cancer such as pancreatic cancer or breat cancer).

The inventors have also identified that murine pancreatic cancer cells exposed to M2-like macrophages upregulate expression of PD-L1 and CD80. Consistent with this, the inventors have found that human PDA tissue samples show an increase in PD-L1 expression compared to controls. Binding of PD-L1 or CD80 with its receptor PD-1 and CTLA-4 respectively leads to functional exhaustion of T cells (reviewed by Pardoll D, nature reviews cancer, 12, 252-264 (April 2012); the blockade of immune checkpoints in cancer immunotherapy). Accordingly, the observed increase in PD-L1 and/or CD80 expression in cancer cells of a subject may attenuate, e.g. suppress and/or inhibit the subject from eliciting an effective immune response.

Novel combinations and pharmaceutical compositions comprising an IGF inhibitor, a chemotherapeutic agent and optionally a PD-L1 inhibitor and/or a CD80 inhibitor, and their use in treating a proliferative disorder such as cancer in a subject are therefore provided. Such combinations and pharmaceutical compositions advantageously counteract the development of resistance to chemotherapy (and optionally resistance to immune response) in such subjects.

In one aspect, the invention provides a pharmaceutical composition comprising a first composition comprising an IGF inhibitor and a pharmaceutically acceptable excipient, adjuvant, diluent or carrier, and a second composition comprising a chemotherapeutic agent and a pharmaceutically-acceptable excipient, adjuvant, diluent or carrier, wherein the IGF inhibitor directly inhibits the biological activity of IGF.

Optionally, the first and second compositions are provided in a form which is suitable for sequential, separate and/or simultaneous administration.

Optionally, the pharmaceutical composition further comprises a PD-L1 and/or CD80 inhibitor.

In another aspect, the invention provides a pharmaceutical composition according to the invention for use in treating a proliferative disorder in a subject.

In another aspect, the invention provides the use of the pharmaceutical composition of the invention in the treatment of a proliferative disorder in a subject.

In another aspect, the invention provides a combination comprising an IGF inhibitor and a chemotherapeutic agent for use in treating a proliferative disorder in a subject, wherein the IGF inhibitor directly inhibits the biological activity of IGF.

Optionally, the combination further comprises a PD-L1 inhibitor and/or a CD80 inhibitor.

In another aspect, the invention provides an IGF inhibitor for use in treating a proliferative disorder in a subject in combination with a chemotherapeutic agent, wherein the IGF inhibitor directly inhibits the biological activity of IGF.

Optionally, the IGF inhibitor is for use in combination with (i) the chemotherapeutic agent and (ii) a PD-L1 inhibitor and/or a CD80 inhibitor.

In another aspect, the invention provides a chemotherapeutic agent for use in treating a proliferative disorder in a subject in combination with an IGF inhibitor, wherein the IGF inhibitor directly inhibits the biological activity of IGF.

Optionally, the chemotherapeutic agent is for use in combination with (i) the IGF inhibitor and (ii) a PD-L1 inhibitor and/or a CD80 inhibitor.

Optionally, in accordance with all aspects of the invention, the proliferative disorder is cancer.

Optionally, in accordance with all aspects of the invention, the cancer is selected from pancreatic cancer, lung cancer, breast cancer, melanoma, colorectal cancer, ovarian cancer, gastric cancer, thyroid cancer, liver cancer, and prostate cancer.

Optionally, in accordance with all aspects of the invention, the proliferative disorder is pancreatic cancer and/or breast cancer.

Optionally, in accordance with all aspects of the invention, a tumor sample isolated from a patient has increased levels of M2-like macrophages (preferably M2-like CD163+ macrophages) compared to a control sample (e.g. normal tissue) or compared to the predetermined reference level.

Optionally, in accordance with all aspects of the invention, said subject is susceptible to developing IGF-induced resistance to said chemotherapeutic agent.

Optionally, in accordance with all aspects of the invention, the IGF inhibitor inhibits at least one IGF selected from IGF-1 and IGF-2.

Optionally, in accordance with all aspects of the invention, the IGF inhibitor inhibits binding of at least one IGF to the insulin receptor, IGFR or hybrid receptors. Further optionally, the IGF inhibitor inhibits binding of IGF-1 to the insulin receptor, IGFR or hybrid receptors and/or inhibits binding of IGF-2 to the insulin receptor, IGFR or hybrid receptors.

Optionally, in accordance with all aspects of the invention, the IGF inhibitor is an anti-IGF antibody or an antigen binding fragment thereof.

Optionally, in accordance with all aspects of the invention, the chemotherapeutic agent is selected from the group consisting of a nucleoside analogue, a topoisomerase inhibitor, a platinum complex, an anti-mitotic agent and combinations thereof. Further optionally, the nucleoside analogue is gemcitabine or fluorouracil and the anti-mitotic agent is paclitaxel

Optionally, in accordance with all aspects of the invention, the IGF inhibitor is an anti-IGF antibody or antigen binding fragment thereof, and wherein the chemotherapeutic agent is gemcitabine, fluorouracil or paclitaxel

Optionally, in accordance with all aspects of the invention, the PD-L1 inhibitor inhibits binding of PDL1 to a PD-1 receptor.

Optionally, in accordance with all aspects of the invention, the PD-L1 inhibitor is an anti-PD-L1 antibody or an antigen binding fragment thereof.

Optionally, in accordance with all aspects of the invention, the CD80 inhibitor inhibits binding of CD80 to a CTLA-4 receptor.

Optionally, in accordance with all aspects of the invention, the CD80 inhibitor is an anti-CD80 antibody or an antigen binding fragment thereof.

In another aspect, the invention provides the use of M2-like macrophages and/or Insulin/IGF receptor activation (measured as expression of phospho-lnsulin/IGF receptors) as biomarkers to select a patient population responsive to or sensitive cancer treatment with an IGF inhibitor and a chemotherapeutic agent.

In another aspect, the invention provides the use of a M2-like macrophages and/or phosphor-lnsulin/IGFR as biomarkers to predict a patient population at risk of chemoresistance to a chemotherapeutic agent.

In another aspect, the invention provides a method of increasing the sensitivity rate (efficacy rate) of a combination of an IGF inhibitor and a chemotherapeutic agent to treat cancer in a patient population said method comprising selecting a sub population expressing M2-like macrophage and/or phospho-lnsulin/IGFR biomarkers.

In another aspect, the invention provides a method of identifying a subject having increased likelihood of responsiveness or sensitivity to a combination of an IGF inhibitor and a chemotherapeutic agent comprising:
a. Determining the level of M2-like macrophages in a tumor sample isolated from the subject; and
b. comparing the level of M2-like macrophages in the patient sample with the level of M2-like macrophages in a control sample (e.g. normal non cancerous tissue) or with a predetermined reference level for M2-like macrophages

wherein an increased level of M2-like macrophages in the tumor sample compared to the control sample or compared to the predetermined reference level is prognostic of an increased likelihood of responsiveness or sensitivity to an IGF inhibitor and a chemotherapeutic agent in the subject.

In another aspect, the invention provides a method of identifying a subject having responsiveness or sensitivity to an IGF inhibitor comprising:
a. Determining the level of M2-like macrophages in a tumor sample isolated from the subject; and
b. comparing the level of M2-like macrophages in the patient sample with the level of M2-like macrophages in a control sample or with a predetermined reference level for M2-like macrophages

wherein an increased level of M2-like macrophages in the tumor sample compared to the control sample or compared to the predetermined reference level is prognostic of an increased likelihood of responsiveness or sensitivity to an IGF inhibitor.

In another aspect, the invention provides a method of treating a subject having cancer comprising:
a. Determining the level of M2-like macrophages in a tumor sample isolated from the subject; and
b. comparing the level of M2-like macrophages in the patient sample with the level of M2-like macrophages in a control sample or with a predetermined reference level for M2-like macrophages
c. administering a therapeutically effective amount of a chemotherapeutic agent and an IGF inhibitor when there is an increased level of M2-like macrophages in the tumor sample compared to the control sample or compared to the predetermined reference level.

Optionally, in accordance with all aspects of the invention, the M2-like macrophage is CD163+.

Optionally, in accordance with all aspects of the invention, the predetermined reference level of M2-like macrophages is at least 20 M2-like macrophages in the tumour core sample.

Optionally, in accordance with all aspects of the invention, the cancer may be pancreatic cancer or breast cancer.

Optionally, in accordance with all aspects of the invention, the chemotherapeutic agent may be gemcitabine, fluorouracil or paclitaxel.

In another aspect, the invention provides a kit for identifying a patient population responsive to a combined treatment with an IGF inhibitor and a chemotherapeutic agent comprising:
a. a detectably labelled agent that specifically binds to a M2-like CD163+ maccophage; and
b. reagents for the assay.

In another aspect, the invention provides an assay device comprising a compound or agent capable of detecting a M2-like macrophage.

In another aspect, the invention provides a pharmaceutical composition as hereinbefore described with reference to the accompanying drawings.

In another aspect, the invention provides a pharmaceutical composition for use in treating a proliferative disorder in a subject as herein before described with reference to the accompanying drawings.

In another aspect, the invention provides the use of the pharmaceutical composition in the treatment of a proliferative disorder in a subject as herein before described with reference to the accompanying drawings.

In another aspect, the invention provides a combination comprising an IGF inhibitor and a chemotherapeutic agent for use in treating a proliferative disorder in a subject as herein before described with reference to the accompanying drawings.

In another aspect, the invention provides an IGF inhibitor for use in treating a proliferative disorder in a subject in combination with a chemotherapeutic agent, as herein before described with reference to the accompanying drawings.

In another aspect, the invention provides a chemotherapeutic agent for use in treating a proliferative disorder in a subject in combination with an IGF inhibitor as herein before described with reference to the accompanying drawings.

### BRIEF DESCRIPTION OF THE DRAWINGS

Embodiments of the invention are further described hereinafter with reference to the accompanying drawings:

FIG. 1 illustrates increased macrophage infiltration in biopsies from PDA patients.

FIG. 2 illustrates increased M2 macrophage infiltration in biopsies from PDA patients.

FIG. 3 illustrates that human macrophages stimulated with human pancreatic cancer cells behave like M2 tumour promoting (immunosuppressive) macrophages.

FIG. 4 illustrates that macrophages promote resistance of pancreatic cancer cells to gemcitabine.

FIG. 5 illustrates that macrophage derived factors promote chemoresistance of pancreatic cancer cells.

FIG. 6 illustrates that macrophages promote chemoresistance of pancreatic cancer cells.

FIG. 7 illustrates that macrophage (M2) derived factors specifically activate Insulin receptor in pancreatic cancer cells.

FIG. 8 illustrates expression of Insulin receptor ligands in human M2-like macrophages.

FIG. 9 illustrates expression levels of IGF-1 in human primary macrophages unstimulated or stimulated with human pancreatic cancer cells in two independent experiments.

FIG. 10 illustrates that IGF-1 expression is increased in murine primary macrophages stimulated with murine KPC tumour cells.

FIG. 11 illustrates that blocking of IGF counteracts macrophage-induced chemoresistance of mouse pancreatic KPC cancer cells and restores their sensitivity to gemcitabine.

FIG. 12 illustrates that blocking of IGF counteracts macrophage-induced chemoresistance of mouse KPC pancreatic cancer cells and restores their sensitivity to gemcitabine.

FIG. 13 illustrates that blocking of IGF restores KPC cells sensitivity to gemcitabine and that recombinant IGF-1 is sufficient to mediate resistance of pancreatic cancer cells to gemcitabine.

FIG. 14 illustrates that blocking of IGF restores tumour cells sensitivity to gemcitabine and that recombinant IGF-1 is sufficient to mediate resistance of pancreatic cancer cells to gemcitabine.

FIG. 15 illustrates that in vivo blockade of IGF in combination with gemcitabine reduces pancreatic tumour growth in mice with pancreatic cancer.

FIG. 16 illustrates that KPC tumour cells exposed to M2 like macrophages up-regulate PDL1 and CD80 expression. Murine pancreatic cancer cells isolated from the genetic KPC (Kras^{G12D/+}; p53^{R172H/+}; Pdx1-Cre) mouse model were cultured in the absence or presence of primary murine macrophage conditioned media for 24 hours. Cells were harvested and stained with anti-PDL1-PE/Cy7, anti-CD80-PE/Cy7 and anti-CD86-PE antibodies from Biolegend. Cells were analysed by flow cytometry for the percentage of cells expressing PDL1, CD80 and CD86. Expression of PDL1 and CD80 was found to be significantly increased in KPC cells when cultured with macrophage conditioned media. However, KPC cells did not seem to express any CD86 in the absence or presence of macrophage conditioned media.

FIG. 17 illustrates that human epithelial cells from malignant pancreatic ducts express high levels of PDL1. Tissue samples from normal pancreas and PDA were stained for PDL1 (using the anti-PDL1 antibody from abcam-ab58810). It was found that PDL1 is expressed in the epithelial malignant pancreatic cancer cells of biopsies from PDA patients.

FIG. 18 illustrates the rationale for combining IGF inhibitors, checkpoint inhibitors (e.g. a PDL-1 inhibitor and/or a CD80 inhibitor) and chemotherapy in the treatment of pancreatic cancer.

FIG. 19 illustrates that IGF blockade partially restores sensitivity of pancreatic cancer cells to 5-FU (fluorouracil).

FIG. 20 illustrates macrophage secreted factors induce chemoresistance in A: human pancreatic Suit-2 cancer cells, B: primary mouse pancreatic cells and C: primary mouse breast cancer cells following culture in the presence or absence of human primary macrophages or macrophage conditioned media (MCM) from human or mouse primary macrophages.

FIG. 21 illustrates increased presence of 1: phospho-Insulin receptor, 2: phospho-AXL receptor and 3: phospho- Ephrin receptor in human pancreatic cancer Suit-2 cells which were serum starved for 24 hours and exposed for 2 hours to human MCM compared to those left unexposed using a phospho-receptor tyrosine kinase array.

FIG. 22 illustrates an immunoblot analysis of phospho-Insulin receptor, Insulin receptor, phospho- IGF receptor 1, IGFR1 and tubulin in Suit-2 cells serum starved or exposed to MCM for 30 min or 3 hours.

FIG. 23 illustrates an immunoblot analysis of pan-phospho tyrosine, Insulin receptor and IGFR1 in Insulin and IGF receptor immunoprecipitates of Suit-2 cells treated with human MCM for 3 hours or left untreated.

FIG. 24 illustrates quantification of IGF-1 and IGF-2 mRNA expression levels in human primary macrophages unexposed or exposed to tumor conditioned media (TCM) from human pancreatic cancer Suit-2 cells.

FIG. 25 illustrates quantification of IGF-1, IGF-2 and Insulin mRNA expression levels in mouse primary macrophages.

FIG. 26 illustrates quantification of IGF-1 and IGF-2 mRNA expression levels in: primary murine macrophages cultured under standard conditions with M-CSF1, M1 polarized macrophages, M2 polarized macrophages, macrophages cultured in the presence of TCM from murine primary Py230 breast cancer cells and from murine primary

KPC pancreatic cancer cells.

FIG. 27 illustrates Immunoblot analysis of phospho-lnsulin/IGF receptor, phospho-Insulin receptor, Insulin receptor, phospho- IGF receptor 1, IGFR1 and tubulin, in human pancreatic cancer Suit-2 cells, murine primary KPC pancreatic cancer and

Py230 breast cancer derived cells, serum starved, exposed to MCM or exposed to recombinant IGF for 3 hours.

FIG. 28 illustrates that blockade of IGF impairs macrophage-mediated activation of Insulin/IGF receptor and downstream signalling molecules IRS1, IRS2 and AKT in cancer cells by Immunoblotting analysis of Suit-2 cells untreated or treated with MCM or MCM+IGF blocking antibody for 3 hours.

FIG. 29 illustrates quantification of cell death in Suit-2 cells treated with gemcitabine, MCM and IGF blocking antibody for 24 hours. Error bars represent s.d. (n=3); **two tailed p value≤0.01, *two tailed p value≤0.05 using a student's t-test.

FIG. 30 illustrates primary mouse KPC-derived pancreatic cancer cells were untreated or treated with gemcitabine, MCM, IGF blocking antibody or recombinant IGF for 24 hours and percentage of cell death was quantified by flow cytometry. Error bars represent s.d. (n=3); **two tailed p value≤0.01 using a student's t-test.

FIG. 31 illustrates representative flow cytometry dot blots of KPC-derived cells exposed to gemcitabine, MCM, IGF blocking antibody and recombinant IGF.

FIG. 32 illustrates KPC-derived cells which were cultured in the presence or absence of MCM or recombinant IGF and treated with 100 or 1000 nM nab-paclitaxel for 24 hours. Percentage of cell death was quantified by flow cytometry. Error bars represent s.d. (n=3); *two tailed p value≤0.05 using a student's t-test.

FIG. 33 illustrates human breast MDA-MB-231 cancer cells cultured in the presence or absence of MCM or recombinant IGF and treated with 500 nM nab-paclitaxel for 48 hours. Percentage of cell death was quantified by flow cytometry. Error bars represent s.d. (n=3); *two tailed p value≤0.05, **two tailed p value≤0.01 using a student's t-test.

FIG. 34 ilustrates that phospho-insulin/IGF receptors confocal microscopy images of frozen human PDA tissues immunofluorescently co- stained for the tumor epithelial marker CK11 (in green), phospho-lnsulin/IGF receptor (in red), and nuclei (in blue) and shows that phospho-insulin/IGF receptors are activated on cancer cells in biopsies from PDA pateients. Scale bar, 100 μm.

FIG. 35 illustrates immunohistochemical staining of phospho- Insulin/IGF receptor in normal human pancreas and biopsies from PDA patients. Scale bars, 100 μm and 50 μm.

FIG. 36 ilustrates a pie diagram representing the percentage of phospho-lnsulin/IGF receptor positive (red) and negative (green) tumors assessed in tissue microarrays (TMA) containing biopsies from 54 different PDA patients.

FIG. 37 illustrates confocal microscopy images of frozen human PDA tissues immunofluorescently co-stained for CD68 (in green), CK19 (in red) and nuclei (in blue). Scale bar, 100 μm.

FIG. 38 illustrates immunohistochemical staining of CD163 in normal human pancreas and biopsies from PDA patients. Scale bars, 100 μm and 50 μm.

FIG. 39 illustrates serial sections of biopsies from PDA patients immunohistochemically stained for phospho- Insulin/IGF receptor and CD163. Scale bar, 100 μm.

FIG. 40 illustrates a contingency table and results from statistical analysis showing a positive correlation between phospho-lnsulin/IGFR expression in tumors and increased M2-like macrophage infiltration. Chi-square =9.272; p=0.002.

FIG. 41 illustrates a contingency table and results from statistical analysis showing a positive correlation between tumors that are both phospho-lnsulin/IGFR positive and highly infiltrated by M2-like macrophages and advanced tumor stage. Fisher Exact Test; p=0.018.

FIG. 42 illustrates serial sections of biopsies from: (a) non-malignant breast tissue immunohistochemically stained for phospho-lnsulin/IGF receptor and CD163. Scale bars, 100 μm and 50 μm. (b) and (c) from breast cancer patients immunohistochemically stained for phospho-lnsulin/IGF receptor and CD163. Scale bars, 100 μm and 50 μm.

FIG. 43 illustrates a histogram depicting the quantification of CD68 and CD163 positive macrophages in normal breast and breast cancer tissue samples. n=6-8 fields counted.

Error bars represent s.d. (n=3); *two tailed p value≤0.05, ***two tailed p value≤0.005 using a student's t-test.

FIG. 44 illustrates a pie diagram representing the percentage of phospho-lnsulin/IGF receptor positive (red) and negative (green) tumors assessed in a tissue microarray containing biopsies from 75 different breast cancer patients.

FIG. 45 illustrates a positive correlation between phospho-lnsulin/IGFR expression in breast cancer tumors and increased M2-like macrophage infiltration.

FIG. 46 illustrates that intra-tumoral macrophages and α-SMA+ stromal cells are the main sources of IGF in pancreatic tumors in vivo (a) KPC-derived tumor cells were orthotopically implanted into the pancreas of syngeneic recipient mice, mimicking PDA. (b) Hematoxilin and Eosin (H&E) and immunohistochemical staining of Ki-67, αSMA, phospho-lnsulin/IGF receptor, CD68 and CD206 staining of naïve mouse pancreas and murine PDA tissue samples harvested at day 30 after tumor implantation. (c) KPC^{luc/zsGreen}(zsGreen)-derived tumor cells were orthotopically implanted into the pancreas of syngeneic recipient mice, mimicking PDA. Tumors were harvested at day 23 after implantation digested and tumor cells, non-immune stromal cells and M1-like and M2-like macrophages were sorted by flow cytometry. (d) Quantification of IGF-1, mRNA expression levels in CD45+/F4/80+/CD206− M1-like macrophages, CD45+/F4/80+/CD206+ M2-like macrophages, CD45−/GFP− non-immune stromal cells and CD45−/GFP+ tumor cells isolated from murine pancreatic tumors. Error bars represent s.d. (n=3). (e) Quantification of IGF-2, mRNA expression levels in CD45+/F4/80+/CD206− M1-like macrophages, CD45+/F4/80+/CD206+ M2-like macrophages, CD45−/GFP− non-immune stromal cells and CD45−/GFP+ tumor cells isolated from murine pancreatic tumors. Error bars represent s.d. (n=3). Error bars represent s.d. (n=3).

FIG. 47 illustrates a combination with gemcitabine with IGF blockade inhibites tumor growth in the syngeneic orthotopic pancreatic cancer model: (a) KPC^{luc/zsGreen}(zsGreen)-derived pancreatic tumor cells were orthotopically implanted into the pancreas of syngeneic recipient mice, and mice were treated, starting at day 7 after tumor implantation, twice a week i.p., with either control IgG antibody, gemcitabine (100 mg/Kg), IGF blocking antibody BI 836845 (100 mg/Kg) or a combination of gemcitabine with IGF blocking antibody. (b) Representative images of tumors and tumor weights are shown (n=6 mice per group). (c) Pancreatic tumors were digested and percentage of intra- tumoral F4/80+ macrophages, Ly6C+/Ly6G− inflammatory monoctyes, Gr1+/CD11b+ neutrophils and myeloid derived suppressor cells (MDSCs) and CD8+ cytotoxic T cells (CTCs), among CD45+ immune cells, were quantified by flow cytometry (n=3). (d) Percentage of intra-tumoral CD206− M1-like macrophages and CD206+M2-like macrophages, among F4/80+ macrophages, were quantified by flow cytometry (n=3). (e) Immunohistochemical staining of phospho-lnsulin/IGFR and cleaved caspase-3 in murine PDA tumors treated with IgG (control), gemcitabine, IGF blocking antibody BI 836845 or gemcitabine+BI 836845. (f) Quantification of cleaved caspase-3 positive apoptotic cells in PDA tumors treated with IgG (control), gemcitabine, IGF blocking antibody BI 836845 or gemcitabine+BI 836845 (n=6 fields counted).

FIG. 48 shows schematics depicting the role of M2-like macrophage and myofibroblasts-derived IGFs in activation of the Insulin/IGFR signaling survival pathway and in mediating chemoresistance of cancer cells.

FIG. 49 illustrates human pancreatic cancer Suit-2 cells cultured in the presence or absence of MCM and treated with 0, 100, 200 and 500 nM gemcitabine for 24 hours.

Percentage of cell death was quantified by flow cytometry. Error bars represent s.d. (n=3); *two tailed p value≤1.05, **two tailed p value≤0.01 using a student's t-test.

FIG. 50 illustrates CD206 and IL-12 RNA expression levels quantified in primary human macrophages that were cultured under standard conditions in the presence of macrophage colony-stimulating factor 1 (M-CSF1), polarized into M1 macrophages or polarized into M2 macrophages.

FIG. 51 illustrates CD206 and IL-12 RNA expression levels quantified in primary murine macrophages that were cultured under standard conditions in the presence of macrophage colony-stimulating factor 1 (M-CSF1), polarized into M1 macrophages or polarized into M2 macrophages.

FIG. 52 illustrates quantification of cell death in Suit-2 cells exposed to 200 nM gemcitabine, MCM and/or IGF blocking antibody (10 μg/ml). Error bars represent s.d. (n=3); **two tailed p value≤0.01 using a student's t-test.

FIG. 53 illustrates cell cycle analysis of primary mouse KPC derived pancreatic cancer cells exposed to MCM, IGF blocking antibody (10 ⊐g/ml) or recombinant IGF (100 ng/ml).

FIG. 54 illustrates confocal microscopy images of frozen human PDA biopsies immunofluorescently co-stained for CK11 (green), phospho-lnsulin/IGF receptor (red) and DNA (blue). Scale bar, 50 μm.

FIG. 55 illustrates immunohistochemical staining of phospho-lnsulin/IGF receptor, CD68 and CD163 in human normal pancreas and serial sections from biopsies of PDA patients.

FIG. 56 illustrates quantification of CD68 and CD163+macrophages in human normal pancreas and PDA samples (n=6-8 fields).

FIG. 57 illustrates clinical information from the 54 PDA samples analysed by immunohistochemistry for phospho-lnsulin/IGFR expression on cancer cells and CD163+ macrophage infiltration.

FIG. 58 illustrates clinical information from the 75 breast cancer samples analysed by immunohistochemistry for phospho-lnsulin/IGFR expression on cancer cells and CD163+ macrophage infiltration.

FIG. 59 illustrates immunofluorescent staining of EpCAM (green), αSMA (red) and nuclei (blue) in frozen tissues from naïve mouse pancreas and mouse pancreatic tumors from orthotopic syngeneic model. Scale bar, 100 μm.

FIG. 60 illustrates immunohistochemical staining of αSMA and CD68 in paraffin embedded tissues from naïve mouse pancreas and mouse pancreatic tumors from orthotopic syngeneic model. Scale bar, 50 μm.

FIG. 61 illustrates immunohistochemical staining of phospho-lnsulin/IGFR, CD206 and αSMA, in serial sections of pancreatic tumor tissues from the genetically engineerd KPC mouse model and in adjacent normal pancreas. Scale bar, 50 μm.

FIG. 62 illustrates: a) KPC-derived tumor cells orthotopically implanted into the pancreas of syngeneic recipient mice, mimicking PDA. Normal pancreas from naïve mice and pancreatic tumorswere harvested and digested on day 29 after implantation. Percentage of intra-tumoral F4/80+ macrophages, Gr1+/CD11b+ neutrophils and myeloid derived suppressor cells (MDSCs), CD4+ and CD8+ T cells, among CD45+ immune cells, were quantified by flow cytometry (n=2-4 mice). (b) Quantification of IGF-1, mRNA expression levels in F4/80+ and F4/80+ cells isolated from murine pancreatic tumors. Error bars represent s.d. (n=3) (c) Quantification of IGF-2, mRNA expression levels in F4/80+ and F4/80− cells isolated from murine pancreatic tumors. Error bars represent s.d. (n=3).

FIG. 63 ilustraes the percentage of intra-tumoral immune cells (among CD45+ cells) in Naïve and Tumor samples.

FIG. 64 illustrates the gating strategy used to sort CD45−/GFP+ KPC-derived tumor cells, CD45−/GFP− non immune stromal cells, CD45+F4/80+/CD206− M1-like macrophages and CD45+/F4/80+/CD206+ M2-like macrophages from pancreatic tumors.

FIG. 65 illustrates the quantification of αSMA mRNA expression levels in the CD45−/GFP− non immune stromal cell population isolated from pancreatic tumors using flow cytometry.

FIG. 66 illustrates a bright field microscopy image of primary pancreatic myofibroblasts isolated from naïve murine pancreas (top). Immunoblotting analysis of αSMA and GAPDH expression in primary pancreatic myofibroblasts (bottom).

FIG. 67 illustrates quantification of IGF-1 mRNA expression levels in primary murine pancreatic myofibroblasts untreated and treated with KPC-derived tumor conditioned media.

FIG. 68 illustrates quantification of IGF-2 mRNA expression levels in primary murine pancreatic myofibroblasts untreated and treated with KPC-derived tumor conditioned media.

FIG. 69 illustrates: (a) Primary mouse KPC-derived pancreatic cancer cells implanted orthotopically in the pancreas of syngeneic recipient mice. Mice were administered i.p., twice a week with IgG antibody, gemcitabine alone or gemcitabine with IGF blocking antibody (ab9572). Tumors were harvested at day 30 and representative images are shown. (b) Tumor weights are shown (n=9-12 mice per group).

FIG. 70 illustrates Immunohistochemical staining of cleaved caspase-3, phospho-Insulin/IGFR, CD206 and CD68 in naïve pancreas, and murine PDA tumors treated with IgG, gemcitabine or gemcitabine+ IGF blocking antibody. Right, Bargraph showing the percentage of apoptotic cancer cells (cleaved caspase-3 positive) quantified in tumor tissues harvested from mice treated with IgG control antibody, gemcitabine or gemcitabine+IGF blocking antibody.

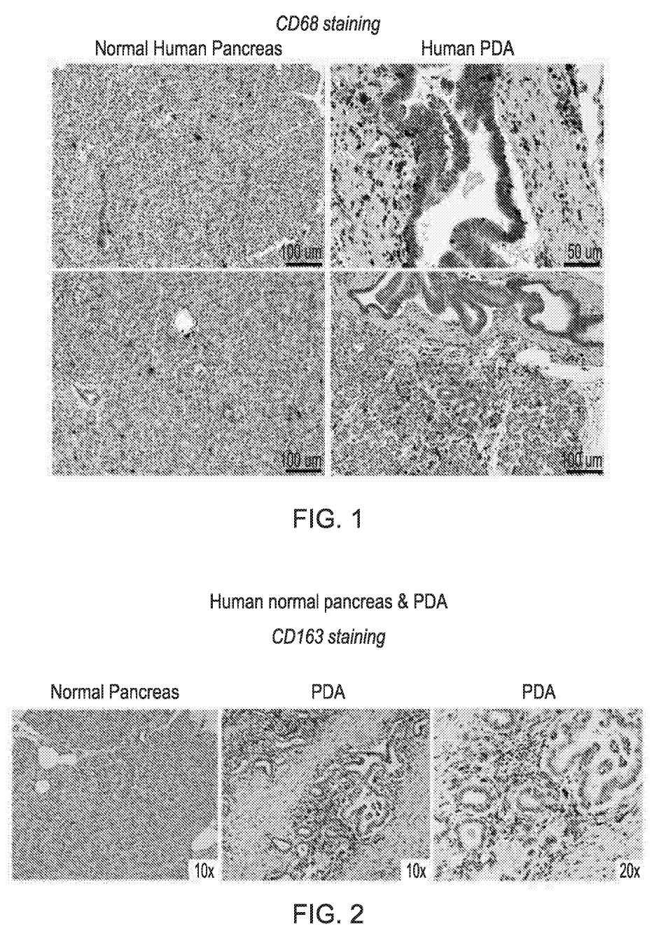

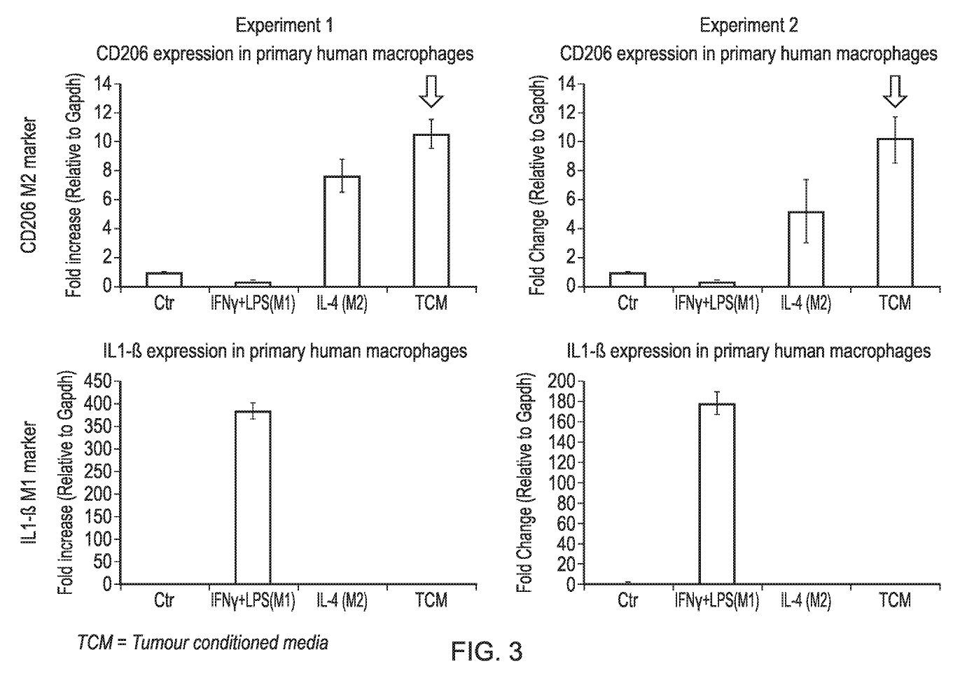

### DETAILED DESCRIPTION

The invention is based on the surprising finding that pancreatic TAMs (but not naïve macrophages) express high levels of Insulin growth factor 1 (IGF-1) and Insulin growth factor 2 (IGF-2) and that pancreatic TAMs are an important source of these growth factors in the tumour microenvironment.

Surprisingly, the inventors have also found that secretion of IGF-1 and IGF-2 by TAMs leads to the activation of Insulin Receptor signaling and the development of cancer cell resistance to chemotherapy (specifically, resistance of pancreatic cancer cells to gemcitabine and/or 5-FU).

Importantly, blockade of IGF activity e.g. IGF-1 and IGF-2 activity (but not IGFR activity) using an IGF blocking antibody restored sensitivity of pancreatic cancer cells to chemotherapy (specifically gemcitabine and/or 5′FU) in vitro and restrained tumour progression and reduced the immuno-suppressive microenvironment in a pre-clinical model of pancreatic cancer.

The findings presented herein suggest that: i) in PDA, IGF ligands rather signal through either Insulin receptor or hybrid Insulin/IGF-R1 receptors but not through IGF-R1 homodimers, which might explain why inhibition of IGFR has failed in pancreatic cancer clinical trials (Basu et al., 2011; Guha, 2013; Pollak, 2012), ii) inhibition of IGF ligands can sensitize pancreatic cancer cells to chemotherapy. Combinatorial treatments using means that directly block IGF ligand activity and chemotherapy therefore provide exciting new therapeutic opportunities to treat proliferative disorders such as PDA.

As TAMs are present in the tumour microenvironment of many cancers, therapeutics that can specifically inhibit the pro-tumoral functions of TAMs, while sparing their anti-tumoral capacity, hold great promise in the goal of restraining cancer progression, metastasis and relapse in more general terms.

The invention therefore provides a new approach to treating a proliferative disorder such as cancer using a combination of compounds, specifically an IGF inhibitor and a chemotherapeutic agent.

The inventors have also identified that murine pancreatic cancer cells exposed to M2-like macrophages upregulate expression of PDL-1 (also known as PDL-1, B7-H1 or CD274) and CD80 (also known as B7-1) and that human PDA tissue samples also show an increase in PDL-1 expression compared to controls. Binding of PDL-1 or CD80 with its receptor PD-1 (also known as CD279) and CTLA-4 (also known as CD152) respectively leads to functional exhaustion of T cells (reviewed by Pardoll D, nature reviews cancer, 12, 252-264 (April 2012); the blockade of immune checkpoints in cancer immunotherapy). Accordingly, the observed increase in PDL-1 and/or CD80 expression in cancer cells of a subject may attenuate, e.g. suppress and/or inhibit the subject from eliciting an effective immune response.

The invention therefore provides a new approach to treating a proliferative disorder such as cancer using a combination of compounds, specifically an IGF inhibitor, a chemotherapeutic agent and (i) a PDL-1 inhibitor and/or (ii) a CD80 inhibitor. The inventors have also surprisingly found that M2-like macrophages and myofibrobalsts are the main sources of Insulin-like growth factors in the tumour microenvironment but do not express insulin and that insulin-IGF receptor activation positively correlates with increased M2-like CD163+macrophage infiltration in tumours and with tumour progression. Thus, the present invention has identified M2-like macrophages and phospho-lnsulin/IGFR as biomarkers and their use as a stratification tool.

Compounds

The invention provides one or more compounds that are suitable for use in treating a proliferative disorder in a subject.

As used herein, the term “compound” is intended to include one or more compounds and refers to a substance that is suitable for use in treating a proliferative disorder in a subject.

By way of example, the compound may be an antibody or at least an antigen binding fragment thereof, an antisense molecule, a polypeptide, a nucleic acid, a carbohydrate, a lipid, a small molecular weight compound, an oligonucleotide, an oligopeptide, siRNA, a recombinant protein, a peptibody, or conjugates or fusion proteins thereof.

Preferably, the compound is an IGF inhibitor, a chemotherapeutic agent, a PDL-1 inhibitor and/or a CD80 inhibitor.

In one particular example, the compound of the invention (e.g. the compound capable of inhibiting IGF, e.g. the IGF inhibitor) is an antibody or at least an antigen binding fragment thereof.

Accordingly, an antibody may be provided, or at least an antigen binding fragment thereof, wherein the antibody or antigen binding fragment is capable of inhibiting IGF (e.g. is an IGF inhibitor). In such an example, the antibody or antigen binding fragment thereof may bind to IGF directly (e.g. specifically).

It is noted that any of the compounds of the invention may be an antibody or at least an antigen binding fragment thereof (i.e. it is not limited to compounds capable of inhibiting IGF). By way of example, but not by way of limitation, a compound that is capable of inhibiting PDL-1 (e.g. a PDL-1 inhibitor) may be an antibody or at least an antigen binding fragment thereof in the context provided herein. By way of a further example, but not by way of limitation, a compound that is capable of inhibiting CD80 (e.g. a CD80 inhibitor) may be an antibody or at least an antigen binding fragment thereof in the context provided herein. Accordingly, the term “IGF” as used below when describing antibodies may equally be replaced with the terms “CD80” or “PDL-1” where the context allows.

The term “antibody” is used herein in its broadest sense and refers to immunoglobulin molecules and immunologically active portions thereof, i.e., molecules that contain an antigen binding site which specifically binds an antigen, such as IGF (e.g. IGF-1 and/or IGF-2), preferably human IGF (e.g. human IGF-1 and/or IGF-2). A molecule which specifically binds to IGF is a molecule which binds IGF (e.g. IGF-1 and/or IGF-2), but does not substantially bind other molecules in a sample, e.g., a biological sample, which naturally contains IGF (e.g. IGF-1 and/or IGF-2).

The antigen may be such as CD80 or PDL-1, preferably CD80 or PDL-1. A molecule which specifically binds to CD80 or PDL-1 is a molecule which binds CD80 or PDL-1, but does not substantially bind other molecules in a sample, e.g., a biological sample, which naturally contains CD80 or PDL-1.

Immunoglobulins (Ig) are a class of structurally related proteins consisting of two pairs of polypeptide chains, one pair of light (L) (low molecular weight) chain (κor λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region.

The H chains of Ig molecules are of several classes: α, μ, σ, ε, and γ (of which there are several sub-classes). An assembled Ig molecule consisting of one or more units of two identical H and L chains, derives its name from the H chain that it possesses. Thus, there are five Ig isotypes: IgA, IgM, IgD, IgE and IgG (with four sub-classes based on the differences in the H chains, i.e., IgG1, IgG2, IgG3 and IgG4). Further detail regarding antibody structure and their various functions can be found in, Using Antibodies: A laboratory manual, Cold Spring Harbour Laboratory Press.

The antibody may be a polyclonal or a monoclonal antibody that binds IGF, CD80 or PDL-1. As used herein, the term “monoclonal antibody” refers to a population of antibody molecules that contain only one species of an antigen binding site capable of immunoreacting with a particular epitope of IGF (e.g. IGF-1 and/or IGF-2), CD80 or PDL-1. A monoclonal antibody composition thus typically displays a single binding affinity for a particular protein with which it immunoreacts. This particular protein may be a class of proteins (such as IGF; which includes IGF-1 and IGF-2) or may be a specific singular protein (such as IGF-1,IGF-2, CD80 or PDL-1).

The antibody may be humanised. A humanised monoclonal antibody to an IGF, CD80 or PDL-1 polypeptide may be produced as a fusion polypeptide in an expression vector suitably adapted for transfection or transformation of prokaryotic or eukaryotic cells. Said antibody may be humanised by recombinant methods to combine the complementarity determining regions of said antibody with both the constant (C) regions and the framework regions from the variable (V) regions of a human antibody.

Optionally, said antibody is provided with a marker including a conventional label or tag, for example a radioactive and/or fluorescent and/or epitope label or tag.

Alternatively, said antibody is a chimeric antibody. Chimeric antibodies are recombinant antibodies in which all of the V-regions of a mouse or rat antibody are combined with human antibody C-regions. Humanised antibodies are recombinant hybrid antibodies which fuse the complementarity determining regions from a rodent antibody V-region with the framework regions from the human antibody V-regions. The C-regions from the human antibody are also used. The complementarity determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not illicit an immune response. This results in a weaker immune response and a decrease in the clearance of the antibody. This is desirable when using therapeutic antibodies in the treatment of diseases. Humanised antibodies are designed to have less “foreign” antibody regions and are therefore thought to be less immunogenic than chimeric antibodies.

The terms “antibody”, “antigen binding fragment” and “at least an antigen binding fragment” are used interchangeably herein unless the context dictates otherwise.

As used herein, an “at least an antigen binding fragment” of an antibody may be an antibody fragment. The antibody fragment may be, but is not limited to, a fab fragment, a F(ab′)2 fragment, or a fragment produced by a fab expression library.

The antibody or antibody fragment may be a monoclonal antibody, a humanised antibody, a chimeric antibody or a single chain antibody, or an epitope binding fragment thereof.

The antibody, or at least an antigen binding fragment thereof (e.g. an antibody fragment), may specifically bind to an IGF polypeptide (e.g. IGF-1 and/or IGF-2), CD80 or PDL-1, preferably a human IGF, CD80 or PDL-1 polypeptide. The molecular structure of IGF-1 and IGF-2, CD80 or PDL-1 is discussed herein in more detail elsewhere.

The inventors have surprisingly found that a compound that is capable of inhibiting IGF, CD80 or PDL-1 (e.g. an IGF, CD80 or PDL-1 inhibitor) is useful in the treatment of a proliferative disorder in a subject in combination with a chemotherapeutic agent because it is capable of inhibiting, reducing and/or preventing resistance to chemotherapeutic agent(s) in the subject.

Compounds that are Capable of Inhibiting IGF, CD80 or PDL-1

As used herein, the phrase “capable of inhibiting IGF, CD80 or PDL-1” refers to (but is not limited to) compounds that have the ability to reduce, inhibit and/or prevent the biological activity of IGF, CD80 or PDL-1. As used herein, the phrases “IGF, CD80 or PDL-1 activity”, “IGF, CD80 or PDL-1 biological activity” and “the biological activity of IGF, CD80 or PDL-1” are used interchangeably to describe the overall mechanism by which IGF, CD80 or PDL-1 carries out their function within a cell (e.g. a cell having or at risk of having a proliferative disorder). A compound that is capable of inhibiting IGF, CD80 or PDL-1 activity may inhibit IGF, CD80 or PDL-1 activity directly (e.g. by interaction with the IGF, CD80 or PDL-1 polypeptide or by interaction with a nucleic acid encoding the IGF, CD80 or PDL-1 polypeptide).

Preferably, a compound that is capable of inhibiting IGF, CD80 or PDL-1 is an IGF, CD80 or PDL-1 inhibitor.

As used herein, an “inhibitor” may be an antibody, a polypeptide, a nucleic acid, a carbohydrate, a lipid, a small molecular weight compound, an oligonucleotide, an oligopeptide, siRNA, antisense, a recombinant protein, a peptibody, or conjugates or fusion proteins thereof.

As used herein, the phrase “IGF, CD80 or PDL-1 inhibitor” refers to an inhibitor that directly inhibits the biological activity of IGF, CD80 or PDL-1 (e.g. by binding to and/or interacting with IGF, CD80 or PDL-1 so as to inhibit the biological activity of IGF, CD80 or PDL-1; by sequestering circulating IGF, CD80 or PDL-1 in the cell; and/or by inhibiting upstream pathways that are required for IGF activity such as IGF, CD80 or PDL-1 expression and/or IGF, CD80 or PDL-1 post-translational modification). For example, the IGF, CD80 or PDL-1 inhibitor may inhibit binding of IGF, CD80 or PDL-1 to its receptor (e.g. the insulin receptor, the IGFR and Insulin/IGFR hybrids, the CD80 receptor or the PDL-1 receptor) through direct interaction with IGF, CD80 or PDL-1.

By way of example only, the IGF, CD80 or PDL-1 inhibitor may be an antibody (or at least an antigen binding fragment thereof) that, when bound to IGF, CD80 or PDL-1, inhibits IGF, CD80 or PDL-1 from binding to its receptor (e.g. the insulin receptor, the IGFR and Insulin/IGFR hybrids the CD80 receptor or the PDL-1 receptor).

As used herein, the terms “inhibit”, “down-regulate”, “prevent” and “reduce” refer to an alteration of the level of IGF , CD80 or PDL-1 biological activity such that the aforementioned level of activity is less than that observed in the absence of the inhibiting substance or compound (e.g. inhibitor). Inhibition may be reversible or irreversible.

A compound that is capable of inhibiting IGF (e.g. the IGF inhibitor) may be capable of inhibiting one or more IGFs (e.g. IGF-1 and/or IGF-2 i.e. it may be an IGF-1 and/or IGF-2 inhibitor).

As used herein, the term “IGF” refers to one or more insulin-like growth factors, which are proteins with high sequence similarity to insulin. The term “IGF” encompasses the two native ligands, IGF-1 and IGF-2, and also natural IGF variants such as brain IGF, otherwise known as des(1-3)IGF-1. The IGF referred to herein may be from any species, including human, murine, bovine, ovine, porcine, and equine.

IGF-1 (insulin-like growth factor 1; also known as somatomedin C) is encoded by the IGF1 gene in humans. IGF-1 undergoes alternative splicing. There are six different isoforms of IGF-1 which share the same mature peptide. Accordingly, the invention is considered equally applicable to all IGF-1 isoforms. The Genbank details for human IGF-1 are shown below:

IGF1
Nucleotide (coding mRNA)

| | |
|---|---|
| LOCUS | NM_001111283 7370 bp mRNA linear PRI |
| | 25 MAY 2014 |
| DEFINITION | Homo sapiens insulin-like growth factor 1 (somatomedin |
| | C) (IGF1), transcript variant 1, mRNA. |
| ACCESSION | NM_001111283 |
| VERSION | NM_01111283.1 GI:163659898 |

Peptide (Amino Acid)

| | |
|---|---|
| LOCUS | CAG46659 153 aa linear PRI 16 OCT. 2008 |
| DEFINITION | IGF1 [Homo sapiens]. |
| ACCESSION | CAG46659 |
| VERSION | CAG46659.1 GI:49456677 |

IGF-1 is an endocrine hormone with a similar molecular structure to insulin. It is mainly secreted by the liver as a result of growth hormone (GH) stimulation. It is required for achieving maximal growth. IGF-I has also been shown to have an involvement in regulating neural development including neurogenesis, myelination, synaptogenesis, and dendritic branching and neuroprotection after neuronal damage. Its primary action is mediated by binding to its specific receptor, the insulin-like growth factor 1 receptor (IGF1R), which is present on many cell types in many tissues. Mutagenesis of IGF-1 has shown Ala8, Asp12, Phe23 and Tyr24 in the B domain; Tyr31, Arg36, Arg37 in the C peptide and Met59, Tyr60 and Ala62 in the A domain to be important for high affinity binding to IGF1R (Meyts and Whittaker, 2002). The C peptide plays a major role in IGF binding to IGF1R.

IGF-1 also binds to the insulin receptor (albeit at significantly lower affinity than it binds to IGF1R). IGF-1 has been shown to bind and interact with all the IGF binding proteins (IGFBPs), of which there are seven: IGFBP1, IGFBP2, IGFBP3, IGFBP4, IGFBP5, IGFBP6, and IGFBP7. Some IGFBPs are inhibitory. For example, both IGFBP-2 and IGFBP-5 bind IGF-1 at a higher affinity than IGF-1 binds the IGF1R. Therefore, increases in serum levels of these two IGFBPs result in a decrease in IGF-1 activity.

IGF-2 (insulin-like growth factor 2) is encoded by the IGF2 gene in humans. IGF-2 undergoes alternative splicing. There are three different isoforms of IGF-2, one of which is known as the canonical isoform and the other two are highly similar. Accordingly, the invention is considered equally applicable to all IGF-2 isoforms. The Genbank details for human IGF-2 are shown below:

IGF2
Nucleotide (coding mRNA)

| | |
|---|---|
| LOCUS: | HUMGFIII 1045 bp mRNA linear PRI 8 NOV. 1994 |
| DEFINITION: | Human insulin-like growth factor II mRNA, complete |
| | cds. |
| ACCESSION: | M29645 |
| VERSION: | M29645.1 GI:183121 |

Peptide (Amino Acid)
insulin-like growth factor II precursor [Homo sapiens]
GenBank: AAA52544.1

| | |
|---|---|
| LOCUS | AAA52544 180 aa linear PRI 8 NOV. 1994 |
| DEFINITION | insulin-like growth factor II precursor [Homo sapiens]. |
| ACCESSION | AAA52544 |
| VERSION | AAA52544.1 GI:183122 |

IGF-2 is also a hormone with similar molecular structure to insulin. It is thought to be a primary growth factor necessary in early development, and is also required for development and function of organs such as the brain, liver and kidney. IGF-2 exerts its effects by binding to the IGF-1 receptor. IGF-2 also binds to the insulin receptor and to the IGF-2 receptor (also called the cation-independent mannose 6-phosphate receptor), which functions as an IGF-2 sequestering agent and thus prevents IGF-2 signaling.

As used herein, an IGF inhibitor that inhibits binding of IGF to its receptor may inhibit binding of IGF (e.g. IGF-1 and/or IGF-2) to one or more receptors selected from IGF1R, IGF2R, insulin receptor and hybrid receptors e.g. hybrid insulin/IGFR1 receptors. Preferably, the one or more receptors comprises the insulin receptor. This is because the inventors have surprisingly found that secretion of IGF-1 (and IGF-2) by TAMs leads to activation of insulin receptor signalling and the development of cancer cell resistance to chemotherapy (specifically, resistance of pancreatic cancer cells to gemcitabine and/or 5′FU), and importantly, that blockade of IGF-1 and IGF-2 activity (but not IGFR activity) using an IGF blocking antibody restored sensitivity of pancreatic cancer cells to gemcitabine and/or 5′FU.

The findings presented herein also indicate that especially high levels of IGF-1 are expressed by pancreatic TAMs and that the presence of recombinant IGF-1 alone is sufficient to induce resistance to gemcitabine in pancreatic cancer cells. Accordingly, a compound capable of inhibiting IGF-1 specifically, without inhibiting IGF-2 and/or other IGFs (e.g. an IGF-1 specific inhibitor such as an anti-IGF-1 antibody or antigen binding fragment thereof) is also considered to be a compound for use in accordance with the invention.

Compounds that are Chemotherapeutic Agents

As used herein, the phrase “chemotherapeutic agent” refers to (but is not limited to) compounds that are used in chemotherapy for the treatment of proliferative disorders such as cancer.

Several chemotherapeutic agents are known, some of which are clinically approved or awaiting approval as cancer therapies. Suitable examples include nucleoside analogues, topoisomerase inhibitors, platinum complexes, and combinations thereof. A specific example of a nucleoside analogue is gemcitabine, although many others are well known.

Another specific example of a nucleoside analogue is fluorouracil (also known as 5-FU). A specific example of a topoisomerase inhibitor is irinotecan, although many others are well known. A specific example of a platinum complex is oxaliplatin, although many others are well known.

Examples of chemotherapeutic agents include alkylating agents such as thiotepa and CYTOXAN(™) cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine;

acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g., calicheamicin, especially calicheamicin (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, ADRIAMYCIN(™) doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amisacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSKO polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL(®) paclitaxel (Bristol-Myers Squibb Oncology, Princeton, N.J.) and TAXOTERE(®) doxetaxel (Rhone-Poulenc Rorer, Antony, France); chlorambucil; GEMZAR(™) gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE(™)0 vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumours such as anti-oestrogens and selective oestrogen receptor modulators (SERMs), including, for example, tamoxifen (including NOLVADEX(™) tamoxifen), raloxifene, droloxifene, 4-hydroxytamoxifen, trioxifene, keoxifene, LY117018, onapristone, and FARESTON(™) toremifene; aromatase inhibitors that inhibit the enzyme aromatase, which regulates estrogen production in the adrenal glands, such as, for example, 4(5)-imidazoles, aminoglutethimide, MEGAS(™) megestrol acetate, AROMASIN(™) exemestane, formestane, fadrozole, RIVISOR(™) vorozole, FEMARA(™) letrozole, and ARIMIDEX(™) anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf, and H-Ras; ribozymes such as a VEGF expression inhibitor (e.g., ANGIOZYME(®) ribozyme) and a HER2 expression inhibitor; vaccines such as gene therapy vaccines, for example, ALLOVECTIN(™) vaccine, LEUVECTIN(™) vaccine, and VAXID(™) vaccine; PROLEUKIN(™) rIL-2; LURTOTECAN(™) topoisomerase I inhibitor; ABARELIX(™) rGnRH; and pharmaceutically acceptable salts, acids or derivatives of any of the above.

The selection of suitable chemotherapeutic agents may depend on the specific proliferative disorder being treated. By way of example, but not by way of limitation, suitable chemotherapeutic agents for use in the treatment of pancreatic cancer include gemcitabine, fluorouracil, FOLFIRINOX (=Leucovorin also known as folinic acid, fluorouracil, Irinotecan and Oxaliplatin) and nab-paclitaxel.

The inventors have identified that a compound that is capable of inhibiting IGF (e.g. an IGF inhibitor) is useful in the treatment of a proliferative disorder in a subject in combination with a chemotherapeutic agent and (i) a compound that is capable of inhibiting PDL-1 (e.g. a PDL-1 inhibitor) and/or (ii) a compound that is capable of inhibiting CD80 (e.g. a CD80 inhibitor). Advantageously, this combination is capable of inhibiting, reducing and/or preventing resistance to chemotherapeutic agent(s) and is capable of inhibiting, reducing and/or preventing suppression of an immune response in the subject.

Compounds that are Capable of Inhibiting PDL-1

As used herein, the phrase “capable of inhibiting PDL-1” refers to (but is not limited to) compounds that have the ability to reduce, inhibit and/or prevent the biological activity of PDL-1. As used herein, the phrases “PDL-1 activity”, “PDL-1 biological activity” and “the biological activity of PDL-1” are used interchangeably to describe the overall mechanism by which PDL-1 carries out its function within a cell (e.g. a cell having or at risk of having a proliferative disorder). A compound that is capable of inhibiting PDL-1 activity may inhibit PDL-1 activity directly (e.g. by interaction with the PDL-1 polypeptide or by interaction with a nucleic acid encoding the PDL-1 polypeptide), or may interfere with PDL-1 binding to its receptor (PD-1), either by directly blocking the interaction between PDL-1 and its receptor or by affecting PD-1 activity such that it inhibits PD-1-mediated PDL-1 activity.

Preferably, a compound that is capable of inhibiting PDL-1 is a PDL-1 inhibitor.

As stated above, an “inhibitor” may be an antibody, a polypeptide, a nucleic acid, a carbohydrate, a lipid, a small molecular weight compound, an oligonucleotide, an oligopeptide, siRNA, antisense, a recombinant protein, a peptibody, or conjugates or fusion proteins thereof.

As used herein, the phrase “PDL-1 inhibitor” refers to an inhibitor that directly inhibits the biological activity of PDL-1 (e.g. by binding to and/or interacting with PDL-1 so as to inhibit the biological activity of PDL-1; by sequestering circulating PDL-1 in the cell; and/or by inhibiting upstream or downstream pathways that are required for PDL-1 activity such as PDL-1 expression, PDL-1 post-translational modification and/or PDL-1 signalling via the PD-1 receptor). For example, the PDL-1 inhibitor may inhibit binding of PDL-1 to its receptor (e.g. the PD-1 receptor) through direct interaction with PDL-1 or through direct interaction with the PD-1 receptor.

By way of example only, the PDL-1 inhibitor may be an antibody (or at least an antigen binding fragment thereof) that, when bound to PDL-1, inhibits PDL-1 from binding to its receptor (e.g. the PD-1 receptor). An example of such an inhibitor is the anti-PDL-1 antibody MEDI 4736.

By way of an alternative example, the PDL-1 inhibitor may be an antibody (or at least an antigen binding fragment thereof) that, when bound to a PD-1 receptor, inhibits PDL-1 from binding to the PD-1 receptor. An example of such an inhibitor is the anti-PD-1 antibody MEDI 0680.

Further examples of agents that target immune-checkpoint pathways are provided in table 1 below, which is reproduced from Pardoll D, Nature Reviews cancer, 12, 252-264 (April 2012):

| | Biological | Antibody or Ig fusion | |
|---|---|---|---|
| Target | function | protein | Slate of clinical development* |
| CTLA4 | Inhibitory | Ipilimumab | FDA approved for melanoma. Phase II and |
| | receptor | | Phase III trials ongoing for multiple cancers |
| | | Tremelimumab | Previously tested in a Phase III trial of patients |
| | | | with melanoma; not currently active |
| PD1 | Inhibitory | MDX-1106 (also known as | Phase I/II trials in patients with melanoma and |
| | receptor | BMS-936558) | renal and lung cancers |
| | | MK3475 | Phase I trial in multiple cancers |
| | | CT-011^{‡} | Phase I trial in multiple cancers |
| | | AMP-224^{§} | Phase I trial in multiple cancers |
| PDL1 | Ligand for PD1 | MDX-1105 | Phase I trial in multiple cancers |
| | | Multiple mAbs | Phase I trials planned for 2012 |
| LAG3 | Inhibitory | IMP321^{∥} | Phase III trial in breast cancer |
| | receptor | Multiple mAbs | Preclinical development |
| B7-H3 | Inhibitory ligand | MGA271 | Phase I trial in multiple cancers |
| B7-H4 | Inhibitory tigand | | Preclinlcal development |
| TIM3 | Inhibitory | | Predinical development |
| | receptor | | |

CTLA4, cytotoxic T-lymphocyte-associated antigen 4; FDA, US Food and Drug Administration; Ig, immunoglobulin; LAG3, lymphocyte activation gene 3; mAbs, monoclonal antibodies; PD1, programmed cell death protein 1; PDL, PD1 ligand; TIM3, T cell membrane protein 3.
*As of January 2012.
^{‡}PD1 specificity not validated in any published material.
^{§}PDL2-Ig fusion protein.
^{∥}LAG3-Ig fusion protein.

As used herein, the terms “inhibit”, “down-regulate”, “prevent” and “reduce” refer to an alteration of the level of PDL-1 biological activity such that the aforementioned level of activity is less than that observed in the absence of the inhibiting substance or compound (e.g. inhibitor). Inhibition may be reversible or irreversible.

As used herein, the term “PDL-1” refers to programmed death ligand-1 (PDL-1), also known as cluster of differentiation 274 (CD274) or B7 homolog 1 (B7-H1). In humans, PDL-1 is encoded by the CD274 gene. The PDL-1 referred to herein may be from any species, including human, murine, bovine, ovine, porcine, and equine.

PDL-1 is a 40kDa type 1 transmembrane protein that is thought to play an important role in suppressing the immune system during particular events such as pregnancy, tissue allografts, autoimmune disease and other disease states such as hepatitis. PDL-1 binds to its receptor, the PD-1 receptor, which is found on activated T cells, B cells, and myeloid cells, to modulate activation or inhibition of these immune cells. By way of example, binding of PDL-1 or CD80 with its receptor PD-1 and CTLA-4 respectively leads to functional exhaustion of T cells (reviewed by Pardoll D, Nature Reviews Cancer 12, 252-264 (April 2012; The blockade of immune checkpoints in cancer immunotherapy). Accordingly, the PDL-1 expression in vivo can act to supress an immune response.

The Genbank details for human PDL-1 are shown below:

PD-L1 = CD274 molecule [Homo sapiens]

| | |
|---|---|
| GenBank: | AAH693811 |
| LOCUS | AAH69381 290 aa |
| DEFINITION | CD274 molecule [Homo sapiens]. |
| ACCESSION | AAH69381 |
| VERSION | AAH69381.1 GI:46854604 |

The Genbank details for human PD-1 are shown below:

PD-1 = CD279 molecule [Homo sapiens]

| | |
|---|---|
| GenBank: | AAH74740.1 |
| LOCUS | AAH74740 288 aa linear PRI 15 JUL. 2006 |
| DEFINITION | Programmed cell death 1 [Homo sapiens]. |
| ACCESSIOIN | AAH74740 |
| VERSION | AAH74740.1 GI:49902307 |

Compounds that are Capable of Inhibiting CD80

As used herein, the phrase “capable of inhibiting CD80” refers to (but is not limited to) compounds that have the ability to reduce, inhibit and/or prevent the biological activity of CD80. As used herein, the phrases “CD80 activity”, “CD80 biological activity” and “the biological activity of CD80” are used interchangeably to describe the overall mechanism by which CD80 carries out its function within a cell (e.g. a cell having or at risk of having a proliferative disorder). A compound that is capable of inhibiting CD80 activity may inhibit CD80 activity directly (e.g. by interaction with the CD80 polypeptide or by interaction with a nucleic acid encoding the CD80 polypeptide), or may interfere with CD80 binding to its receptor (CTLA-4), either by directly blocking the interaction between CD80 and its receptor or by affecting CD80 activity such that it inhibits CTLA-4-mediated CD80 activity.

Preferably, a compound that is capable of inhibiting CD80 is a CD80 inhibitor.

As stated above, an “inhibitor” may be an antibody, a polypeptide, a nucleic acid, a carbohydrate, a lipid, a small molecular weight compound, an oligonucleotide, an oligopeptide, siRNA, antisense, a recombinant protein, a peptibody, or conjugates or fusion proteins thereof.

As used herein, the phrase “CD80 inhibitor” refers to an inhibitor that directly inhibits the biological activity of CD80 (e.g. by binding to and/or interacting with CD80 so as to inhibit the biological activity of CD80; by sequestering circulating CD80 in the cell; and/or by inhibiting upstream or downstream pathways that are required for CD80 activity such as CD80 expression, CD80 post-translational modification and/or CD80 signalling via the CTLA-4 receptor). For example, the CD80 inhibitor may inhibit binding of CD80 to its receptor (e.g. the CTLA-4 receptor) through direct interaction with CD80 or through direct interaction with the CTLA-4 receptor.

By way of example only, the CD80 inhibitor may be an antibody (or at least an antigen binding fragment thereof) that, when bound to CD80, inhibits CD80 from binding to its receptor (e.g. the CTLA-4 receptor). An example of such an inhibitor is the anti-CD80 antibody [1G10] (ab25208) (Abcam).

The CD80 inhibitor may be an antibody (or at least an antigen binding fragment thereof) that acts as a CTLA-4 blocking antibody, e.g. when bound it inhibits CD80 from binding to the CTLA-4 receptor. An example of such an inhibitor is the anti-CTLA-4 antibody tremelimumab or Ipilimumab.

As used herein, the terms “inhibit”, “down-regulate”, “prevent” and “reduce” refer to an alteration of the level of CD80 biological activity such that the aforementioned level of activity is less than that observed in the absence of the inhibiting substance or compound (e.g. inhibitor). Inhibition may be reversible or irreversible.

As used herein, the term “CD80” refers to cluster of differentiation 80 (CD80), also known as B7-1. The CD80 referred to herein may be from any species, including human, murine, bovine, ovine, porcine, and equine. CD80 is found on activated B cells and monocytes and provides the costimulatory signal that is necessary for T cell activation and survival. It is the ligand for two different receptors found on T cells, namely CD28 and CTLA-4.

The Genbank details for human CD80 are shown below:

CD80 molecule [Homo sapiens]

| | |
|---|---|
| GenBank: | AAH42665.1 |
| LOCUS | AAH42665 288 aa linear PRI 8 SEP. 2006 |
| DEFINITION | CD80 molecule [Homo sapiens]. |
| ACCESSION | AAH42665 |
| VERSION | AAH42665.1 GI:27503576 |

The Genbank details for human CTLA-4 are shown below:

CTLA-4 (Cytotoxic T-lymphocyte-associated
protein 4) [Homo sapiens]

| | |
|---|---|
| GenBank: | AAH69566.1 |
| LOCUS | AAH69566 223 aa linear PRI 15 JUL. 2006 |
| DEFINITION | Cytotoxic T-Iymphocyte-associated protein 4 |
| | [Homo sapiens]. |
| ACCESSION | AAH69566 |
| VERSION | AAH69566.1 GI:46854814 |

M2-like Macrophages

The present inventors have surprisingly shown that M2 but not M1-like macrophages directly support chemoresistance of cancer cells by secreting Insulin-like growth factors (IGFs), which activate Insulin/IGF receptors on cancer cells. Immunohistochemical analysis of biopsies from pancreatic and breast cancer patients revealed that 57% and 75% of the patients respectively express activated Insulin/IGF receptors, and this positively correlates with increased M2-like macrophage infiltration and tumor progression. The preent inventors surprising found that, in vivo, both M2-like macrophages and aSMA+ stromal cells are the main sources of IGF production.

Accordingly, the present invention is predicated on the surprising finding that M2-like macrophages and phospho-lnsulin/IGFR can be used as biomarkers to identify patient poulations which may be sensitive to a combination of an IGF inhibitor and a chemotherapeutic agent.

In another aspect, the invention provides the use of a M2-like macrophage and phospho-Insulin/IGFR as biomarkers to select a patient population responsive to or sensitive cancer treatment with an IGF inhibitor and a chemotherapeutic agent.

In another aspect, the invention provides the use of a M2-like macrophage and phospho-Insulin/IGFR as biomarkers to predict select a patient population at risk of chemoresistance to a chemotherapeutic agent.

In another aspect, the invention provides a method of increasing the sensitivity rate (efficacy rate) of a combination of an IGF inhibitor and a chemotherapeutic agent to treat cancer in a patient population said method comprising selecting a sub population having a M2-like macrophage and/or phospho-lnsulin/IGFR biomarker.

In another aspect, the invention provides a method of identifying a subject having increased likelihood of responsiveness or sensitivity to a combination of an IGF inhibitor and a chemotherapeutic agent comprising:
a. Determining the level of M2-like macrophages and/or phospho-lnsulin/IGFR in a tumor sample isolated from the subject; and
b. comparing the level of M2-like macrophages and/or phospho-lnsulin/IGFR in the patient sample with the level of M2-like macrophages and/or phospho-lnsulin/IGFR in a control sample or with a predetermined reference level for M2-like macrophages

wherein an increased level of M2-like macrophages and/or phospho-lnsulin/IGFR in the tumor sample compared to the control sample or compared to the predetermined reference level is prognostic of an increased likelihood of responsiveness or sensitivity to an IGF inhibitor and a chemotherapeutic agent in the subject.

In another aspect, the invention provides a method of identifying a subject having responsiveness or sensitivity to an IGF inhibitor comprising:
a. Determining the level of M2-like macrophages and/or phospho-lnsulin/IGFR in a tumor sample isolated from the subject; and
b. comparing the level of M2-like macrophages and/or phospho-lnsulin/IGFR in the patient sample with the level of M2-like macrophages in a control sample or with a predetermined reference level for M2-like macrophages

wherein an increased level of M2-like macrophages and/or phospho-lnsulin/IGFR in the tumor sample compared to the control sample or compared to the predetermined reference level is prognostic of an increased likelihood of responsiveness or sensitivity to an IGF inhibitor.

In another aspect, the invention provides a method of treating a subject having cancer comprising:
a. Determining the level of M2-like macrophages and/or phospho-lnsulin/IGFR in a tumor sample isolated from the subject; and
b. comparing the level of M2-like macrophages and/or phospho-lnsulin/IGFR in the patient sample with the level of M2-like macrophages and/or phospho-lnsulin/IGFR in a control sample or with a predetermined reference level for M2-like macrophages
c. administering a therapeutically effective amount of a chemotherapeutic agent and an IGF inhibitor when there is an increased level of M2-like macrophages and/or phospho-Insulin/IGFR in the tumor sample compared to the control sample or compared to the predetermined reference level.

In another aspect, the invention provides a kit for identifying a patient population which would benefit from a combined treatment with an IGF inhibitor and a chemotherapeutic agent comprising:
a. a detectably labelled agent that specifically binds to a M2-like CD163+ macrophage and to phospho-lnsulin/IGFR; and
b. reagents for the assay.

In another aspect, the invention provides an assay device comprising a compound or agent capable of detecting a M2-like macrophage and phospho-lnsulin/IGFR.

Optionally, in accordance with all aspects of the invention, the M2-like macrophage is CD163+.

Optionally, in accordance with all aspects of the invention, the predetermined reference level of M2-like macrophages is at least 20 or at least 30 or at least 40 or at least 50 M2-like macrophages in the tumour core sample.

Optionally, in accordance with all aspects of the invention a scoring may be allocated to the levels of M2-like macrophages in a tumour sample isolated from a patient. The scorings may be as follows None=0 macrophages/core, Low=up to about 30 macrophages/core, Medium=31-50 macrophages/core, High=51 and above macrophages/core. Optionally, in accordance with all asepcts of the invention the predetermind reference level may differentiate no or low macrophage levels from medium/high levels.

Optionally, in accordance with all aspects of the invention a core sample may be approximately 1.5 mm.

Optionally, in accordance with all aspects of the invention, the chemotherapeutic agent is gemcitabine, fluorouracil, paclitaxel or nab-paclitaxel.

As used herein “patient” refers to an individual, e.g., a human, having a proliferative disorder such as cancer.

As used herein the term “labeled”, refers to direct labeling of the probe or antibody by coupling (i.e., physically linking) a detectable substance to the probe or antibody, as well as indirect labeling of the probe or antibody by reactivity with a detectable substance.

The term “prognosis” is used herein to refer to the prediction of the likelihood of cancer-attributable death or progression, including, for example, recurrence, metastatic spread, and drug resistance, of a neoplastic disease, such as cancer.

The term “prediction” or (and variations such as predicting) is used herein to refer to the likelihood that a patient will respond either favorably or unfavorably to a drug or set of drugs. In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent (e.g. a chemotherapeutic agent) or combination (e.g. an IGF inhibitor in combination with a chemotherapeutic agent), chemotherapy, etc. or whether long-term survival of the patient, following a therapeutic regimen is likely.

The term “increased resistance” to a particular therapeutic agent or treatment option, when used in accordance with the invention, means decreased response to a standard dose of the drug or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity of treatment.

“Responsive to cancer treatment ”and “sensitive toe cancer treatment” can be assessed using any endpoint indicating a benefit to the patient, including, without limitation, (1) inhibition, to some extent, of tumor growth, including slowing down or complete growth arrest; (2) reduction in the number of tumor cells; (3) reduction in tumor size; (4) inhibition (e.g., reduction, slowing down or complete stopping) of tumor cell infiltration into adjacent peripheral organs and/or tissues; (5) inhibition (e.g., reduction, slowing down or complete stopping) of metastasis; (6) enhancement of antitumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

The term “prediction” or (and variations such as predicting) is used herein to refer to the likelihood that a patient will respond either favorably or unfavorably to a drug or set of drugs (e.g. the combination of an IGF inhibitor and a chemotherapeutic agent). In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, chemotherapy, etc. , or whether long-term survival of the patient, following a therapeutic regimen is likely.

The level of M2-like macrophage in a sample may be determined by techniques known in the art, such as enzyme linked immunosorbent assays (ELISAs), immunoprecipitation, immunofluorescence, Immunohistochemistry, enzyme immunoassay (EIA), radioimmunoassay (RIA), and Western blot analysis. For in vivo detection of an M2-like macrophages a labeled anti-M2-like (e.g. CD163+) macrophage antibody may be introduced into a patient. Such an antibody can be labeled with a radioactive marker whose presence and location in a subject can be detected by standard imaging techniques.

Altenatively, M2-like macrophages could be used as a non-invasive biomarker. For example, M2-like macrophages could be detected in a biological sample (e.g.blood) and analysis could ebe undertaken to detmine if such M2-like macrophages express IGF.

The level of M2-like macrophage in a sample may also be determined by determining the level of M2-like macrophage activity in a sample.

Methods of the invention further comprise comparing the level or activity of M2-like macrophage in the patient (e.g. core) sample with the level or activity of M2-like macrophage in a control sample or with a predetermined reference level for M2-like macrophage.

In one embodiment, methods of the invention include contacting a control sample with a compound or agent capable of detecting an M2-like macrophage nucleic acid molecule, such as mRNA, or genomic DNA, and comparing the level of the M2-like macrophage nucleic acid molecule in the control sample with the level of M2-like macrophage molecule in the patient sample.

In another embodiment, the methods of the invention further include contacting the control sample with a compound or agent capable of detecting an M2-like macrophage, and comparing the level of M2-like macrophage in the control sample with the presence of M2-like macrophage in the test sample.

As used herein “reference level” or “control”, refers to a sample having a normal level of M2-like macrophage expression, for example a sample from a healthy subject not having or suspected of having a proliferative disorder such as cancer. Alternatively, the reference level may be comprised of an M2-like macrophage level from a reference database, which may be used to generate a pre-determined cut off value, i.e. a diagnostic score that is statistically predictive of a symptom (e.g. chemoresistance) or disease or lack thereof or may be a pre-determined reference level based on a standard population sample.

Alternatively, predictions may be based on the normalized expression level of M2-like macrophage. Expression levels are normalized by correcting the absolute expression level of M2-like macrophage in a sample by comparing its expression to the expression of a reference nucleic acid that is not a marker, e.g., an mRNA, such as an mRNA that is constitutively expressed. This normalization allows the comparison of the expression level in one sample to another sample, or between samples from different sources. This normalized expression can then optionally be compared to a reference level or control.

In one embodiment the diagnostic, predictiveor stratification methods involve determining the level of M2-like macrophage in a sample and determining the level of at least one further biomarker, for example a biomarker predictive or indicative associated with chemoresistance. Preferably, the at least one further biomarker may be selected from Insulin/IGFR. Preferably, the level of the at least one further biomarker is determined using any one of the above mentioned methods.

The level of at least one further biomarker may be determined in the same biological sample or a different biological sample to the level of M2-like macrophage.

Alternatively, the level of M2-like macrophage in a sample can be detected and quantified using mass spectrometry.

In one aspect the invention includes an assay device, for example a solid support such as an array or a chip, that has attached to a surface thereof a compound or agent capable of detecting an M2-like macrophage. Preferably, compound or agent capable of detecting an M2-like macrophage is an anti- M2-like macrophage antibody, more preferably an M2-like macrophage capture antibody. In one embodiment the assay device further comprises at least one additional compound or agent for detecting a further biomarker, preferablylnsulin/IGFR.

The methods of the invention allow a skilled person to make informed treatment decisions on the basis of M2-like macrophage. For example, the methods of diagnosis and prognosis described herein may further comprise a step of treating a patient or organ, on the basis of the diagnosis or prognosis. The step of treating a patient or organ may, by way of example only, be administration of a therapeutically effective amount of an IGF inhibitor and a chemotherapeutic agent. The combination may be administered simultaneously or sequentially.

Kits

The invention also includes kits for detecting the presence of M2-like macrophage in a biological sample. For example, the kit can include a compound or agent capable of detecting an M2-like macrophage in a biological sample. The compound or agent can be packaged in a suitable container. The kit can further comprise instructions for using the kit to detect M2-like macrophage.

In one aspect the invention provides a kit for diagnosing determining the treatment strategy for a patient with cancer comprising: a detectably labelled agent that specifically binds to M2-like macrophage; and
ii) reagents for performing a diagnostic assay.

The agent may be an antibody or a nucleic acid molecule. Suitably, the labelled agent may be an anti-CD163 agent that binds to macrophages.

For antibody-based kits, the kit can include: (1) a first antibody (e.g., attached to a solid support) which specifically binds to an M2-like macrophage corresponding to a marker of the invention; and, optionally, (2) a second, different antibody which binds to either the M2-like macrophage or the first M2-like macrophage antibody and is conjugated to a detectable agent.

For oligonucleotide-based kits, the kit can include: (1) a nucleotide probe, e.g., a detectably labeled primer, which hybridizes to an M2-like macrophage or (2) a pair of primers for amplifying a M2-like macrophage.

In one aspect the invention provides a kit for diagnosing ischaemia-reperfusion injury or

[symbol]

The kits can also include components necessary for detecting the detectable agent (e.g., an enzyme or a substrate). The kits can also contain a control sample or a series of control samples which can be assayed and compared to the test sample contained. Suitably, the kits and assay devices of the present invention maty be used to determining tunour stage,to predict tumour prognosis and/or to predict responsiveness to treatment.

Compound Combinations and Compositions

A compound of the invention can be for use or administration alone or in combination with at least one or more other compounds. Administration “in combination with” at least one or more other compounds includes separate, simultaneous (concurrent) and sequential (consecutive) administration in any order. “Combined use” and “combination” in the context of the invention also includes a product comprising both the compound and at least one or more other compounds, as discrete separate dosage forms, in separate containers or e. g. in blisters containing both types of drugs in discrete solid dosage units, e.g. in a form in which the dosage units which have to be taken together or which have to be taken within one day are grouped together in a manner which is convenient for the patient. Said product itself or as a part of a kit may contain instructions for the simultaneous, sequential or separate administration of the discrete separate dosage units, to a subject. Accordingly, the product may comprise at least two compounds (e.g. an IGF inhibitor and a chemotherapeutic agent; and optionally a PDL-1 inhibitor and/or a CD80 inhibitor) as discrete separate dosage forms, in a form which is suitable for sequential, separate and/or simultaneous administration.

The separate formulations of e.g. an IGF inhibitor and a chemotherapeutic agent (and optionally a PDL-1 inhibitor and/or a CD80 inhibitor) may therefore be administered sequentially, separately and/or simultaneously. For example, the separate formulations of an IGF inhibitor and a chemotherapeutic agent (and optionally a PDL-1 inhibitor and/or a CD80 inhibitor) of the combination product may be administered simultaneously (optionally repeatedly). For example, the separate formulations of an IGF inhibitor and a chemotherapeutic agent (and optionally a PDL-1 inhibitor and/or a CD80 inhibitor) of the combination product may be administered sequentially (optionally repeatedly). For example, the separate formulations of an IGF inhibitor and a chemotherapeutic agent (and optionally a PDL-1 inhibitor and/or a CD80 inhibitor) of the combination product may be administered separately (optionally repeatedly). Where the administration of the separate formulations of an IGF inhibitor and a chemotherapeutic agent (and optionally a PDL-1 inhibitor and/or a CD80 inhibitor) of the combination product is sequential or separate, the delay in administering the second formulation (and optionally third formulation) should not be such as to lose the beneficial effect of the combination therapy.

By way of example, a combination is provided comprising an IGF inhibitor and a chemotherapeutic agent (and optionally a PDL-1 inhibitor and/or a CD80 inhibitor). The combination is useful for the treatment of a proliferative disorder.

By way of an alternative example, a pharmaceutical composition is provided comprising a first composition comprising an IGF inhibitor and a pharmaceutically acceptable excipient, adjuvant, diluent and/or carrier, and a second composition comprising a chemotherapeutic agent and a pharmaceutically acceptable excipient, adjuvant, diluent and/or carrier. The first and second compositions may be provided in a form which is suitable for sequential, separate and/or simultaneous administration.

In one example, a pharmaceutical composition is provided comprising a first composition comprising an IGF inhibitor and a pharmaceutically acceptable excipient, adjuvant, diluent and/or carrier, a second composition comprising a chemotherapeutic agent and a pharmaceutically acceptable excipient, adjuvant, diluent and/or carrier, and a third composition comprising a PDL-1 inhibitor and/or a CD80 inhibitor and a pharmaceutically acceptable excipient, adjuvant, diluent and/or carrier. The first, second and third compositions may be provided in a form which is suitable for sequential, separate and/or simultaneous administration.

A compound of the invention may thus be part of a composition (e.g. a pharmaceutical composition) that comprises the compound and one or more other components. A composition may be a pharmaceutical composition that comprises a compound of the invention and a pharmaceutically acceptable excipient, adjuvant, diluent and/or carrier. Pharmaceutical compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected compound without causing any undesirable biological effects or interacting in a deleterious manner with any of the other components of the pharmaceutical composition in which it is contained.

Excipients are natural or synthetic substances formulated alongside an active ingredient (e.g. a compound of the invention), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

The compound combinations and/or compositions of the present invention may be used as a sole therapy or may involve additional surgery or radiotherapy or an additional chemotherapeutic agent or a therapeutic antibody in addition. Such chemotherapeutic agents may include one or more of the following categories of anti-tumour agents:
(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras signalling inhibitors such as farnesyl transferase inhibitors, for example sorafenib (BAY 43-9006)), inhibitors of cell signalling through AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindo1-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications W097/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment.

Treatment of a Subject

The inventors have surprisingly found that a compound that is capable of inhibiting IGF (e.g. an IGF inhibitor) is useful in the treatment of a proliferative disorder in a subject in combination with a chemotherapeutic agent because it is capable of reducing and/or preventing resistance to chemotherapeutic agent(s) in the subject.

Furthermore, the inventors have identified that the above combination may be supplemented with a compound capable of inhibiting PDL-1 (e.g. a PDL-1 inhibitor) and/or a compound capable of inhibiting CD80 (e.g. a CD80 inhibitor) because such compounds are capable of reducing and/or preventing suppression of the immune response in the subject.

As used herein, the terms “treat”, “treating” and “treatment” are taken to include an intervention performed with the intention of preventing the development or altering the pathology of a disorder or symptom. Accordingly, “treatment” refers to both therapeutic treatment and prophylactic or preventative measures, wherein the object is to prevent or slow down (lessen) the targeted pathological disorder or symptom.

As used here in the term “subject” refers to an individual, e.g., a human, pig, horse, mouse, cow, rat etc having or at risk of having a proliferative disorder e.g. cancer. Optionally, the subject is a subject having or at risk of having cancer, e.g. a solid cancer such as pancreatic cancer. The subject may be a patient i.e. a subject in need of treatment in accordance with the invention. The subject may have received treatment for the disorder. Alternatively, the subject has not been treated prior to treatment in accordance with the present invention.

As used herein, the terms “disease” and “disorder” are used interchangeably.

As used herein, the phrase “a proliferative disorder” is intended to include cancer, for example cancer of the lung, head and neck, brain, colon, rectum, oesophagus, stomach, liver, biliary tract, thyroid, kidney, cervix, ovary, uterus, skin, breast, bladder, prostate, pancreas and including haematological malignancies such as leukaemia, multiple myeloma and lymphoma. In particular, “a proliferative disorder” is intended to encompass solid tumours (cancers) (e.g. primary and recurrent solid tumours) such as pancreatic cancer, lung cancer, breast cancer, melanoma, colorectal cancer, ovarian cancer, gastric cancer, thyroid cancer, liver cancer and prostate cancer. A proliferative disorder of particular interest in the context of the invention is pancreatic cancer.

The compounds, combinations and/or compositions described herein can be administered to the subject by any conventional route, including injection or by gradual infusion over time. The administration may, for example, be topical, oral, parenteral, intravenous, intraperitoneal, intramuscular, intravascular, intracavity, intranasal, intracerebral, intratracheal, intralesional, intraperitoneal, intratumoural, rectal, subcutaneous, transdermal, epidural, percutaneous, or by infusion.

The compounds, combinations and/or compositions described herein may be in a form suitable for the above modes of administration. For example, suitable forms for oral administration include a tablet or capsule; suitable forms for nasal administration or administration by inhalation include a powder or solution; suitable forms for parenteral injection (including intravenous, subcutaneous, intramuscular, intravascular or infusion) include a sterile solution, suspension or emulsion; suitable forms for topical administration include an ointment or cream; and suitable forms for rectal administration include a suppository. Alternatively, the route of administration may be by direct injection into, for example, the tumour, or by regional delivery or by local delivery.

The compounds, combinations and/or compositions described herein are for administration in an effective amount. An “effective amount” (or “therapeutically effective amount”) is an amount that alone, or together with further doses, produces the desired (therapeutic) response. The (therapeutically) effective amount to be used will depend, for example, upon the therapeutic objectives, the route of administration, and the condition of the patient. For example, the dosage of the IGF inhibitor and/or the chemotherapeutic agent for a given patient will be determined by the attending physician, taking into consideration various factors known to modify the action of drugs including severity and type of disease, body weight, sex, diet, time and route of administration, other medications and other relevant clinical factors. The dosages and schedules may be varied according to the particular disease state and the overall condition of the patient. For example, it may be necessary or desirable to reduce the above-mentioned doses of the components of the combination treatment in order to reduce toxicity. Therapeutically effective dosages may be determined by either in vitro or in vivo methods.

The compositions of the present invention are advantageously presented in unit dosage form.

The compounds, combinations and/or compositions described herein are therefore administered to a subject in an effective amount to produce the desired response. Examples of such responses include, but are not limited to, anti-tumour effects, the response rate, the time to disease progression and the survival rate. Anti-tumour effects associated with treatment as described herein include but are not limited to, inhibition of tumour growth, tumour growth delay, regression of tumour, shrinkage of tumour, increased time to regrowth of tumour on cessation of treatment, slowing of disease progression. It is expected that when a compound, combination and/or composition described herein is administered to a subject in need of treatment for cancer, said treatment will produce an effect, as measured by, for example, one or more of: the extent of the anti-tumour effect, the response rate, the time to disease progression and the survival rate. Anti-cancer effects include prophylactic treatment as well as treatment of existing disease.

The invention therefore also provides a method of treating a proliferative disorder such as cancer, which comprises administration of a compound, combination and/or composition according to the invention to a subject having or suspected of having said proliferative disorder. Optionally, the cancer is selected from pancreatic cancer, lung cancer, breast cancer, melanoma, colorectal cancer, ovarian cancer, gastric cancer, thyroid cancer, liver cancer and prostate cancer.

Accordingly, in vivo methods of treatment are provided, which may be prophylactic and/or therapeutic.

Preferably, the combination(s) and/or composition(s) described herein will provide a beneficial or synergistic effect on the treatment or prophylaxis of a proliferative disorder such as cancer. A combination treatment is defined as affording a “synergistic effect” or a “synergistic treatment” if the effect is therapeutically superior, as measured by, for example, the extent of the response, the response rate, the time to disease progression or the survival period, to that achievable on dosing one or other of the components of the combination treatment at its conventional dose. For example, the effect of the combination treatment is synergistic if the effect is therapeutically superior to the effect achievable with the IGF inhibitor alone or chemotherapeutic agent alone. Further, the effect of the combination is synergistic if a beneficial effect is obtained in a group of subjects that does not respond (or responds poorly) to the IGF inhibitor or the chemotherapeutic agent alone. In addition, the effect of the combination treatment is defined as affording a synergistic effect if one of the components is dosed at its conventional dose and the other component is dosed at a reduced dose and the therapeutic effect, as measured by, for example, the extent of the response, the response rate, the time to disease progression or the survival period, is equivalent to or better than that achievable on dosing conventional amounts of either one of the components of the combination treatment. In particular, synergy is deemed to be present if the conventional dose of the IGF inhibitor or the chemotherapeutic agent may be reduced without detriment to one or more of the extent of the response, the response rate, the time to disease progression and survival data, in particular without detriment to the duration of the response, but with fewer and/or less troublesome side-effects than those that occur when conventional doses of each component are used.

Predictive Methods

The data provided herein indicate that PDA patients with high levels of IGF will not respond well to gemcitabine and/or 5′FU (i.e. such patients display IGF-induced resistance to chemotherapy). Accordingly, patient IGF levels may be used as a predictive biomarker for response to gemcitabine and/or 5′FU. Use of IGF as a predictive marker would advantageously allow PDA patients with high levels of IGF to be selected for combinatorial treatment in accordance with the invention (i.e. treatment with a combination of chemotherapy and IGF inhibitor, with an optional addition of (i) a PDL-1 inhibitor and/or (ii) a CD80 inhibitor).

The invention therefore provides a method for determining the resistance of a patient with a proliferative disorder to treatment with a chemotherapeutic agent, said method comprising:
(i) measuring IGF protein levels present within a microtumour environment within the patient; and
(ii) comparing the measurement of (i) to control IGF protein levels (e.g. derived from a non-microtumour environment within said patient)

wherein an increase in IGF protein levels in (i) compared to control IGF protein levels is predictive of resistance to treatment with a chemotherapeutic agent.

The invention also provides a method for determining whether a patient with a proliferative disorder will benefit from combinatorial treatment with a chemotherapeutic agent and an IGF inhibitor, said method comprising:
(i) measuring IGF protein levels; IGFR levels; and/or M2-like macrophage levels present within a microtumour environment within the patient; and
(ii) comparing the measurement of (i) to control IGF protein levels; IGFR levels; and/or M2-like macrophage levels (e.g. derived from a non-microtumour environment within said patient)

wherein an increase in IGF protein levels; IGFR levels; and/or M2-like macrophage levels in (i) compared to control IGF protein levels; IGFR levels; and/or M2-like macrophage levels predicts that said patient will benefit from combinatorial treatment with a chemotherapeutic agent and an IGF inhibitor.

### EXAMPLE 1

Introduction

Pancreatic ductal adenocarcinoma (PDA) is an extremely lethal disease of the exocrine pancreas for which novel therapeutic strategies are urgently needed. Drug resistance is one of the biggest challenges in cancer therapeutics and the cause of relapse in the majority of cancer patient, including pancreatic cancer patients. Therefore, understanding the molecular mechanisms of drug resistance is critical to the development of durable treatment strategies.

Although multiple factors can contribute to the resistance of PDA to therapies, one dominant player is the presence of a rich pro-tumoral microenvironment. Tumour associated macrophages (TAMs) are key drivers of this pro-tumoral microenvironment and promote PDA progression and resistance to chemotherapy. However, the molecular basis of how tumour associated macrophages promote resistance of pancreatic cancer cells to chemotherapy remains elusive. In these studies, the inventors found that tumour associated macrophages support resistance to pancreatic cancer cells to chemotherapy by secreting IGF. Mechanistically, our data suggest that macrophage derived IGF leads to Insulin receptor activation on pancreatic cancer cells, which consequently leads to resistance to chemotherapy. Importantly, blockade of IGF restored response of tumours to gemcitabine in pre-clinical models of pancreatic cancer and decreased PDA progression. Accordingly, biopsies from PDA patients show a strong correlation between phospho-Insulin levels, increased numbers of TAMs and poor response to gemcitabine. These findings suggest that specific inhibition of IGF in combination with gemcitabine holds great promise in the treatment of PDA.

Materials and Methods

Cell Culture, Generation of Primary Pancreatic Cancer Cells, Macrophages and Conditioned Media

KPC cells were isolated in our laboratory from PDA tumour tissues obtained from Kras^{G12D/+}; p53^{R172H/+}; Pdx1-Cre mice as described in (Torres et al., PLOS ONE, 2013). Human pancreatic Suit-2 cells and murine KPC cells were cultured in DMEM supplemented with 10% FBS. Murine and human primary isolated monocytes were differentiated into macrophages in M-CSF media and polarised to M1, M2 or cancer associated macrophages with IFN ⊐/LPS, IL-2 or tumour conditioned media respectively. To generated conditioned media, macrophages and pancreatic cancer cells were cultured in serum free DMEM for 24 h, supernatant was harvested, filtered with 0.45 ⊐m filter and stored at −20° C.

Purification of Myeloid Cells

Murine and human myeloid cells were purified from murine bone marrow or human buffy coats obtained from the University of Liverpool Blood Bank (healthy volunteers) using anti-CD11b magnetic bead affinity chromatography according to manufacturer's directions (Miltenyi Biotec). To assess the purity of the CD11b+ cell population, allophycocyanin-labelled anti-CD11 b antibodies was added to cells, and flow cytometry was performed. Cell were >95% CD11b+.

Treatment with Gemcitabine and IGF-1 Blocking Antibody

Suit-2 and KPC cells were cultured in serum free or DMEM with 2% FBS respectively, pretreated for 3 h with human or mouse macrophage conditioned media respectively, and IGF-1 IgG blocking antibody (abcam 9572) at 10 μg/ml or IgG antibody as control, followed by Gemcitabine at 200 nM. Cells were harvested after 24 hours, and subjected to flow cytometry. Cells were stained with annexinV conjugated to FITC and PI (eBioscience) following the manufacturer's protocol. Apoptosis was evaluated by flow cytometry (FACS Calibur, Becton Dickinson).

RTK Arrays and Immunoblotting

Cells were serum starved or treated with macrophage conditioned media for 30 min, 2 h or 3 h, harvested and lysed in RIPA buffer (150 mM NaCl, 10 mM Tris-HCl pH 7.2, 0.1% SDS, 1% Triton X-100, 5 mM EDTA) supplemented with a complete protease inhibitor mixture (SIGMA), a phosphatase inhibitor cocktail (Invitrogen), 1 mM PMSF and 0.2 mM Na₃VO₄. Cell lysates (300 μg) were analyzed with the Human Phospho-RTK Array Kit (R&D Systems). Protein samples were resuspended in 4× Laemmli sample buffer, and immunoblotting was performed on nitrocellulose membranes blocked in 5% BSA in Tris-buffered saline Tween-20. Membranes were incubated with antibodies in 5% milk or 5% bovine serum albumin overnight at 4° C.: anti-pInsulin/pIGFR1 (R&D), anti-pinsulin (LSBioScience), anti-tubulin (Sigma), anti-Insulin (abcam), anti-Insulin (R&D) anti-pIGF1R, anti-IGF1R diluted to 1:1000.

Orthotopic Pancreas Cancer Syngeneic Model

KPC primary pancreatic carcinoma cells (1×10⁶ tumour cells in 30 μl of matrigel) were implanted into the pancreas of six- to eight-week-old female isogenic PC recipient mice obtained from Cambridge, as previously described (Qiu et al., Methods Mol Biol. 2013ref). Tumours were established for 2 weeks before beginning treatment. Mice were administered i.p with Gemcitabine (125 mg/kg), IGF-1 blocking antibody (abcam 9572) 25 μg/mouse or IgG isotype control antibody every 2-3 days for 15 days before harvest. At endpoint, tumours were harvested, photographed, weighed, cryopreserved in OCT, fixed in formalin, solubilized for RNA purification, or collagenase-digested for flow cytometric analysis of immune cell populations (CD11 b, Gr1, CD206, CD8, Treg expression. Phospho-Insulin levels, immune cell infiltration and apoptosis was measured by phospho-insulin (R&D and LSBioscience), CD68 (DAKO), CD206 (Abcam), CD8 Abcam) and cleaved caspase 3 (Cell signalling) immunofluorescent and immunohistochemical staining, composition of immune cell populations was assessed on digested tumours by flow cytometry and CD206, collagen and IGF-1 expression levels were determined by qPCR. All in vivo experiments were performed with n=8-10 mice.

Gene Expression

Total RNA was isolated from cells and tissues using ISOGEN (Nippon Gene). cDNA was prepared from 1 μg RNA/sample, and qPCR was performed using gene specific QuantiTect Primer Assay primers from Qiagen. Relative expression levels were normalized to gapdh expression according to the formula <2̂−(Ct gene of interest−Ct gapdh)>. Fold increase in expression levels were calculated by comparative Ct method <2A−(ddCt)>.

Quantification of Immune Cells in Tissues by Flow Cytometry

To quantify the different immune cell populations in tissues, tumours were excised, minced and digested to single cell suspensions for 1 h at 37° C. in 5 ml of Hanks Balanced Salt Solution (HBSS, GIBCO) containing 1 mg/ml Collagenase type IV, 10 μg/ml Hyaluronidase type V and 20 units/ml DNase type IV (Sigma). Red blood cells were solublized with RBC Lysis Buffer (eBioscience) and then cells were incubated in FC-blocking reagent (BD Bioscience), followed by anti-CD45-PE/Cy7 (Biolegend), anti-CD11b-FITC (Biolegend), anti-Gr1-APC/Cy7 (Biolegend), anti-F4/80-APC (Biolegend), anti-CD206-PerCP/Cy5.5 (Biolegend), anti-CD8-PerCP/Cy5.5 (Biolegend), anti-CD3-FITC (Biolegend), anti-CD4-APC (Biolegend). To exclude dead cells, 0.5 μg/ml propidium iodide was added.

Immunohistochemical Analysis

Deparaffinization and antigen retrieval was performed using an automated DAKO PT-link. Paraffin-embedded human and mouse PDA tumours were immunostained using the DAKO envision+system-HRP. Tissue sections were incubated overnight at 4° C. with primary antibodies against phospho-Insulin (R&D) (1:50), phospho-Insulin (LSBios) (1:100), CD68 (DAKO) (1:1000), CD163 (abcam) (pre-diluted), F4/80 (affymetrix) (1:100), aSMA (abcam) (1:100), CD8 (abcam) (1:100). Staining was developed using diamino-benzidine and counterstained with hematoxylin.

Immunofluorescence Analysis

Human and mouse PDA frozen tissue sections were fixed with cold acetone, permeabilized in 0.1% Triton, blocked in 8% goat serum and incubated overnight at 4° C. with anti-phospho Insulin (R&D) (1:200), anti-phospho-Insulin (LSBio) (1:100), CD68 (DAKO, clone KP1) 1:200, CK19 (abcam) (1:200), CD8 (Biolegend) (1:100), F4/80 (Biolegend) (1:100), CD206 (abcam) (1:100), a SMA (abcam) (1:100), EpCAM (BD Pharmingen) (1:100) and cleaved caspase 3 (Cell signaling) (1:300). The appropriate fluorescently labelled secondary antibodies were co-incubated with the DNA dye TOPRO-3 (Invitrogen).

Statistical Methods

All error bars indicate standard error of the mean (SEM) of 3-5 replicates (in vitro studies) or 8-10 replicates (animal studies). Statistical significance was assessed with the Student t test for in vitro studies and ANOVA coupled with posthoc tests for in vivo studies using SigmaXL 6.02. A value of P<0.05 was considered significant and signified by an asterisk.

Institutional Approvals

All studies involving human tissues were approved by the University of Liverpool and were considered exempt according to national guidelines. All experiments on live animals were performed in accordance with institutional and national guidelines and regulations, under approval by the University of Liverpool BSU.

Results and Discussion

Pancreatic ductal adenocarcinoma (PDA) is one of the most lethal diseases worldwide. Current therapies are unfortunately in most cases ineffective. Drug resistance is one of the biggest challenges in cancer therapeutics and the cause of relapse in the majority of cancer patient, including pancreatic cancer patients (Zahreddine and Borden, 2013). Therefore, understanding the molecular mechanisms of resistance is critical to the development of durable treatment strategies.

Despite extensive efforts invested in the clinical development of therapies against PDA, current standard treatments only include chemotherapeutic agents highly toxic and of modest efficacy (Li et al., 2004). Thus, the development of more effective and less toxic therapies to treat PDA is an urgent need. Although multiple factors can contribute to the resistance of PDA to therapies, one dominant player is the presence of a rich pro-tumoral microenvironment (Gore and Korc, 2014; Lunardi et al., 2014; McMillin et al., 2013; Mielgo and Schmid, 2013; Neesse et al., 2013; Noy and Pollard, 2014; Weizman et al., 2014). Tumour associated macrophages (TAMs) are key drivers of this pro-tumoral microenvironment and can promote tumour cell proliferation, drug resistance and metastasis in many carcinomas including PDA (Noy and Pollard, 2014; Ruffell et al., 2014). Thus, high numbers of TAMs often correlate with resistance to chemotherapy and metastasis leading to poor survival in pancreatic cancer patients (Ino et al., 2013; Kurahara et al., 2011). Previous studies from our group and others have shown that inhibition of myeloid cell infiltration into the tumour restrains cancer progression (DeNardo et al., 2011; Schmid et al., 2011a; Schmid et al., 2011b; Schmid et al., 2013; Zhu et al., 2014) However, TAMs can be polarised into M1 inflammatory/anti-tumoral macrophages that will activate an immune response against the tumour or M2 immunosuppressive/pro-tumoral macrophages that promote tumour immunity (Kurahara et al., 2011; Mielgo and Schmid, 2013; Ruffell et al., 2012). Thus, therapeutics that can specifically inhibit the pro-tumoral functions of TAMs, while sparing their anti-tumoral capacity, hold great promise in the goal of restraining PDA progression, metastasis and relapse.

The inventors have investigated the mechanisms by which pro-tumoral TAMs impact PDA resistance to chemotherapy and tumour progression.

The inventors have demonstrated that pancreatic TAMs (but not naïve macrophages) express high levels of IGF-1 (and are the main source of these growth factors in the tumour microenvironment). Secretion of IGF by TAMs consequently lead to the activation of Insulin Receptor signaling and cancer cells resistance to chemotherapy. The inventors also found that phospho-Insulin receptor is highly expressed in epithelial malignant pancreatic ductal cells in biopsies from PDA patients and that increased phosphorylation of Insulin Receptor on cancer cells correlates with PDA progression and response to gemcitabine. Importantly, blockade of IGF ligands (but not IGFR) restored sensitivity of pancreatic cancer cells to chemotherapy in vitro and restrained tumour progression and reduced the immuno-suppressive microenvironment in a pre-clinical model of pancreatic cancer. These findings suggest that: i) in PDA, IGF ligands rather signal through either Insulin receptor or hybrid Insulin/IGF-R1 receptors but not through IGF-R1 homodimers, which might explain why inhibition of IGFR has failed in pancreatic cancer clinical trials (Basu et al., 2011; Guha, 2013; Pollak, 2012), ii) inhibition of IGF ligands can sensitize pancreatic cancer cells to chemotherapy. Thus, these studies strongly suggest that combinatorial treatments using IGF ligand blockade with chemotherapy may open new therapeutic opportunities to treat this devastating disease.

Summary Statements

TAMs promote resistance of pancreatic cancer cells to chemotherapy.

A phospho-screen revealed that TAMs specifically trigger the activation of Insulin Receptor (but not IGF-1 Receptor) in pancreatic cancer cells.

TAMs express high levels of IGF.

Phospho-Insulin is highly expressed in pancreatic tumour cells in biopsies from PDA patients.

In vitro blockade of IGF (ligands) inhibits macrophage-induced chemoresistance of pancreatic cancer cells.

In vivo blockade of IGF (ligand) in combination with Gemcitabine decreases tumour growth in a preclinical model of pancreatic cancer.

PD-L1 is highly expressed in pancreatic tumour cells in biopsies from PDA patients.

PD-L1 and CTLA-4 ligand (CD80) are upregulated in pancreatic cancer cells when exposed to TAMs.

These findings suggest that combinatorial treatment using:
1. IGF blocking agents with chemotherapy or
2. IGF blocking agents +Checkpoint inhibitors (i.e. PD-L1/PD-1, CTLA-4 inhibitors) with chemotherapy could hold great promise in the treatment of PDA.

### EXAMPLE 2

Introduction

In these studies, we found that M2 but not M1-like macrophages directly support chemoresistance of cancer cells by secreting Insulin-like growth factors (IGFs), which activate Insulin/IGF receptors on cancer cells. Immunohistochemical analysis of biopsies from pancreatic and breast cancer patients revealed that 57% and 75% of the patients respectively express activated Insulin/IGF receptors, and this positively correlates with increased M2-like macrophage infiltration and tumor progression. In vivo, we found that both M2-like macrophages but also αSMA+ stromal cells are the main sources of IGF production. Importantly, IGF blockade sensitized pancreatic tumors to gemcitabine treatment. These findings suggest that inhibition of IGFs in combination with chemotherapy could benefit cancer patients.

Materials and Methods

Cell Culture, Generation of Primary Pancreatic and Breast Cancer Cells, Macrophages and Conditioned Media

KPC cells were isolated in our laboratory from PDA tumor tissues obtained from KrasG12D/+; p53R172H/+; Pdx1-Cre mice as described previously (Olive et al., Science, 2009). Briefly, a 3 mm³ fragment of PDA was excised, washed in 10 ml PBS, and then finely diced with sterile razors. Cells were incubated in 10 ml collagenase type V solution (1 mg/ml in DMEM/F12) at 37C for 30-45 minutes with mixing. Cells were centrifuged (300+g) and resuspended in 0.05% Trypsin/EDTA for 5 min at 37C. Digest was quenched by adding DMEM+ 10% fetal bovine serum and 96 uM CaCl2. Cells were washed 3 times with DMEM/F12 medium and plated in Biocoat dishes (Collagen I) in the ductal cell medium described previously (Schreiber et al., Gastroenterology, 2004). Cells were maintained on collagen coated plates at a minimal passage number (<p8) to allow initial expansion prior to use for in vitro and in vivo experiments.

Py230 cells were obtained from spontaneously arising tumors in MMTV-PyMT C57BI/6 female mice by serial trypsinization and limiting dilution (Gibby et al., 2012). The mouse model used for obtaining these tumors has been described in detail previously (Guy et al., 1992) and (Davie et al., 2007). Cells were maintained in Ham's F12K nutrient culture media (Life Technologies) with 5% HyClone fetal clone II (GE Life Sciences) and supplemented with MITO serum extender (BD Biosciences). Py230 cells were cultured until confluent and then passaged. Once confluent, they formed well-differentiated colonies (Biswas et al., 2014; Gibby et al., 2012)

Human pancreatic cancer Suit-2 cells and breast cancer MDA-MB-231 cells were cultured in DMEM supplemented with 10% FBS. Human and murine primary isolated monocytes were differentiated into macrophages in DMEM +10% FBS +10 ng/ml recombinant murine M-CSF media or 50 ng/ml recombinant human M-CSF, respectively, followed by polarizing to M1 (20 or 50 ng/ml INFγ and 100 or 10 ng/ml LPS), M2 (20 or 50 ng/ml IL-4 ng/ml) respectively, or tumor associated macrophages (using tumour conditioned media).

To generate conditioned media from macrophages and pancreatic cancer cells, also referred to in these studies as macrophage and tumor derived factors, cells were cultured in serum free DMEM for 24 h, supernatant was harvested, filtered with 0.45pm filter and stored at −20° C.

Generation of Primary Macrophages

Primary murine macrophages were generated by flushing the bone marrow from the femur and tibia of C57BL/6 or mixed 129/SvJae/C57BI/6 (PC) mice followed by incubation for 5 days in DMEM containing 10% FBS and 10 ng/mL murine M-CSF (Peprotech). For some experiments, differentiated macrophages were polarized into an M1, M2, or tumor educated phenotype using INFg (20 ng/ml, Peprotech) and LPS (100 ng/ml, Sigma Aldrich); IL-4(20 ng/ml, Peprotech); or tumor conditioned media, respectively. Primary human macrophages were generated by purifying CD14+ monocytes from blood samples obtained from control healthy subjects using magnetic bead affinity chromatography according to manufacturer's directions (Miltenyi Biotec) followed by incubation for 5 days in DMEM containing 10% FBS and 50 ng/mL recombinant human M-CSF (Peprotech). Studies were approved by the National Research Ethics (Research Integrity and Governance Ethics committee- Reference: RETH000807). All individuals provided informed consent for blood donation on approved institutional protocols.

Treatment with Chemotherapy, IGF Blocking Antibody and Recombinant IGF

Suit-2, KPC, cells were cultured in serum free or DMEM with 2% FBS respectively, pretreated for 3 hours with human or isogenic mouse macrophage derived factors (MDF) or recombinant IGF (Peprotech 100-11) at 100 ng/ml, and IGF-1/2 IgG blocking antibody (abcam 9572) at 10 μg/ml or irrelevant IgG antibody as control, followed by Gemcitabine at 200 nM or 10-100nm of nab-paclitaxel. Cells were harvested after 24 hours, and subjected to flow cytometry. Cells were stained with annexinV conjugated to FITC and PI (eBioscience) following the manufacturer's protocol. Cell death was evaluated by flow cytometry (FACS Calibur, Becton Dickinson).

MDA-MB-231 cells were pretreated for 3 hours with human macrophage derived factors (MDF) or recombinant IGF (100 ng/ml) followed by 500 nM of nab-paclitaxel.

Py230 cells were pretreated for 3 hours with isogenic mouse macrophage derived factors (MDF) from M1 or M2 polarized macrophages followed by 100 nM of paclitaxel.

Cell Cycle Analysis

KPC derived pancreatic cancer cells were treated with isogenic mouse MDFs, IGF1/2 blocking antibody (abcam 9572) at 10 μg/ml or recombinant IGF (Peprotech 100-11) at 100 ng/ml. Cells were harvested, fixed with methanol, treated for 45 min with 10 μg/ml of RNase, resuspended in PBS containing 10 μg/ml of propidium iodide and subjected to flow cytometry.

RTK Arrays and Immunoblotting

Cells were serum starved or treated with macrophage conditioned media for 30 min, 2 h or 3 h, harvested and lysed in RIPA buffer (150 mM NaCl, 10 mM Tris-HCl pH 7.2, 0.1% SDS, 1% Triton X-100, 5 mM EDTA) supplemented with a complete protease inhibitor mixture (SIGMA), a phosphatase inhibitor cocktail (Invitrogen), 1 mM PMSF and 0.2 mM Na₃VO₄. Cell lysates (300 μg) were analyzed with the Human Phospho-RTK Array Kit (R&D Systems). Protein samples were resuspended in 4xLaemmli sample buffer, and immunoblotting was performed on nitrocellulose membranes blocked in 5% BSA or milk (depending on the antibody) in Tris-buffered saline Tween-20 (TBST). Membranes were incubated with primary antibodies overnight at 4° C.: anti-pInsulin/pIGFR1 (R&D, AF2507 used 1:400 in 5% Milk-TBST), anti-pinsulin (LSBioScience, LS-C177981 used 1:1000 and repare in 2.5% BSA-TBST), anti-tubulin (Sigma, T6199 used 1:5000 in 2.5% BSA-TBST), anti-Insulin (abcam, 137747 used 1:1000 in 2.5% BSA-TBST), anti-Insulin (R&D, AF1544 used 1:2000 in 2.5% BSA-TBST), anti-pIGF1R (Biorbyt, orb97626 used 1:1000 in 2.5% BSA-TBST), anti-IGF1R (R&D, AF305-NA used 1:1000 in 2.5% BSA-TBST).

Densitometry analysis of immunoblots was performed using ImageJ software and adjusted using the loading control.

Syngeneic Orthotopic Pancreatic Cancer Model.

All animal experiments were performed in accordance with current UK legislation under an approved project licence (reference number: 403725). Mice were housed under specific pathogen-free conditions at the Biomedical Science Unit at the University of Liverpool. Orthotopic pancreatic tumors were initiated by implanting 1×10⁶ primary KPC cells into the pancreas of immune-competent syngeneic mice. Syngeneic recipient mice were six to eight-week-old female mice descended from mice used to generate the KPC PDAC cell lines but lacking oncogeneic KrasG12D expression (PC mice). Tumors were established for 2 week before beginning treatment. Mice were administered i.p with Gemcitabine (100 mg/kg), IGF-1/2 blocking antibody (abcam 9572) 25 μg/mouse or IgG isotype control antibody every 2-3 days for 15 days before harvest. At endpoint, tumors were harvested, photographed, weighed, cryopreserved in OCT, fixed in formalin, solubilized for RNA purification, or collagenase-digested for flow cytometry analysis of immune cells and macrophages (CD45+ and F4/80+ cells respectively). Tumor tissues were analyzed by immunohistochemical and immunofluorescent staining for phospho-lnsulin/IGFR expression, EpCAM expression, macrophage infiltration, fibroblast activation and apoptosis using the following antibodies: phospho-insulin/IGF receptor antibody (R&D), EpCAM (BD Pharmingen), CD68 antibody (DAKO), CD206 (abcam), αSMA (abcam) and cleaved caspase 3 (Cell signaling). Expression of IGF-1, IGF-2, Arginase, IL-10, IFNγ and IL-12 in intra-tumoral isolated macrophages were determined by qPCR. In vivo experiments was performed with n=9-12 mice.

Gene Expression

Total RNA was isolated from purified cells as described for Qiagen Rneasy protocol. Total RNA from entire pancreatic tumor tissues was extracted using a high salt lysis buffer (Guadidine thiocynate 5 M, sodium citrate 2.5 uM, lauryl sarcosine 0.5% in H₂O) to improve RNA quality followed by purification using Qiagen Rneasy protocol.cDNA was prepared from1 μg RNA/sample, and qPCR was performed using gene specific QuantiTect Primer Assay primers from Qiagen. Relative expression levels were normalized to gapdh expression according to the formula <2̂−(Ct gene of interest−Ct gapdh) ((Schmittgen and Livak, 2008). In some cases values were multiplied by 100 for presentation purposes.

Quantification of Immune Cells in Tissues by Flow Cytometry

Single cell suspensions from murine livers were prepared by mechanical and enzymatic disruption in Hanks Balanced Salt Solution (HBSS) with 1 mg/mL Collagenase P (Roche). Cells suspension were centrifuged for 5 min. at 1200 rpm, resuspended in HBSS and filtered through a 500 μm polypropylene mesh (Spectrum Laboratories). Cell suspension was resuspended in 1 mL 0.05%Trypsin and incubated at a 37° C. for 5 minutes. Cells were filtered through a 70 μm cell strainer and resuspended in PBS+5% BSA. Cells were blocked for 10 minutes on ice with FC Block (BD Pharmingen, Clone 2.4G2) and then stained with Sytox viability marker (Life Technologies) and conjugated antibodies against anti-CD45-PE/Cy7 (Biolegend, clone 30-F11), anti-F4/80-APC (Biolegend, clone BM8). Flow Cytometry was performed on a FACSCanto II (BD Biosciences). Part of the single cell suspension was used to isolated tumor associated macrophages using anti-F4/80 magnetic microbeads (Miltenyi Biotechnology) following the manufacturer's protocol.

Immunohistochemical Analysis

Deparaffinization and antigen retrieval was performed using an automated DAKO PT-link. Paraffin-embedded human and mouse PDA tumors were immunostained using the DAKO envision+ system-HRP. Tissue sections were incubated for 1 hour at room temperature with primary antibodies against phospho-Insulin (R&D, AF2507, used 1:50 after high pH antigen retrieval), CD68 (DAKO, clone KP1, M081401-2 used 1:2000 after high pH antigen retrieval) CD163 (abcam, ab74604 pre-diluted after low pH antigen retrieval), αSMA (abcam, Ab 5694 used 1:100 after low pH antigen retrieval), CD206 (abcam, ab8919 used 1:50 after low pH antigen retrieval) followed by secondary-HRP conjugated antibody (from DAKO envision kit) for 30 minutes at room temperature. All antibodies were prepared in antibody diluent from Dako envision kit. Staining was developed using diamino-benzidine and counterstained with hematoxylin.

Immunofluorescence

Human and mouse PDA frozen tissue sections were fixed with cold acetone, permeabilized in 0.1% Triton, blocked in 8% goat serum and incubated overnight at 4° C. with anti-phospho Insulin/IGFR (R&D) (1:200), CD68 (DAKO, clone KP1) (1:200), CK19 (abcam) (1:200), αSMA (abcam) (1:100), EpCAM (BD Pharmingen) (1:100) and cleaved caspase 3 (Cell signaling) (1:300). The appropriate fluorescently labelled secondary antibodies were co- incubated with the DNA dye TOPRO-3 (Invitrogen).

Statistical Methods

All error bars indicate standard deviations of 3-5 replicates (in vitro studies) or 9-12 replicates (animal studies). Statistical significance was assessed with the Student t test or ANOVA coupled with posthoc tests using GraphPad Prism 5. A value of P<0.05 was considered significant and signified by an asterisk. A value of P<0.01 was considered highly significant and signified by two asterisks.

Institutional Approvals

All studies involving human tissues were approved by the University of Liverpool and were considered exempt according to national guidelines. Human pancreas carcinoma samples were obtained from the Liverpool Tissue Bank and patients consented to use the surplus material for research purposes. All experiments on live animals were performed in accordance with institutional and national guidelines and regulations, under approval by the University of Liverpool BSU.

Results and Discussion

Macrophage Derived IGFs Activate Insulin Receptor Signaling on Cancer Cells and Induce Chemoresistance

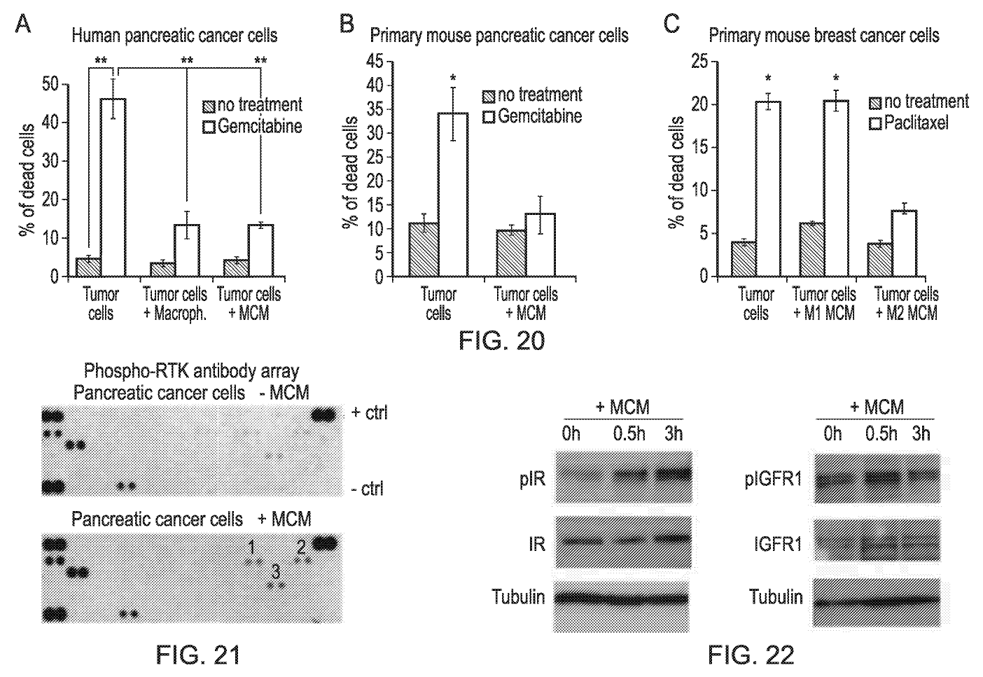

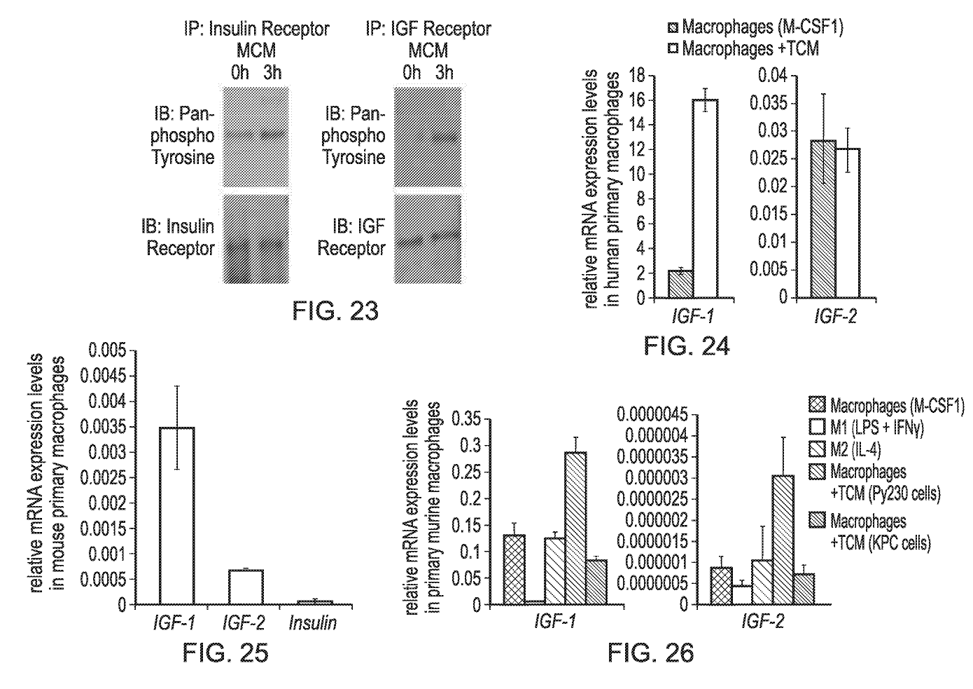

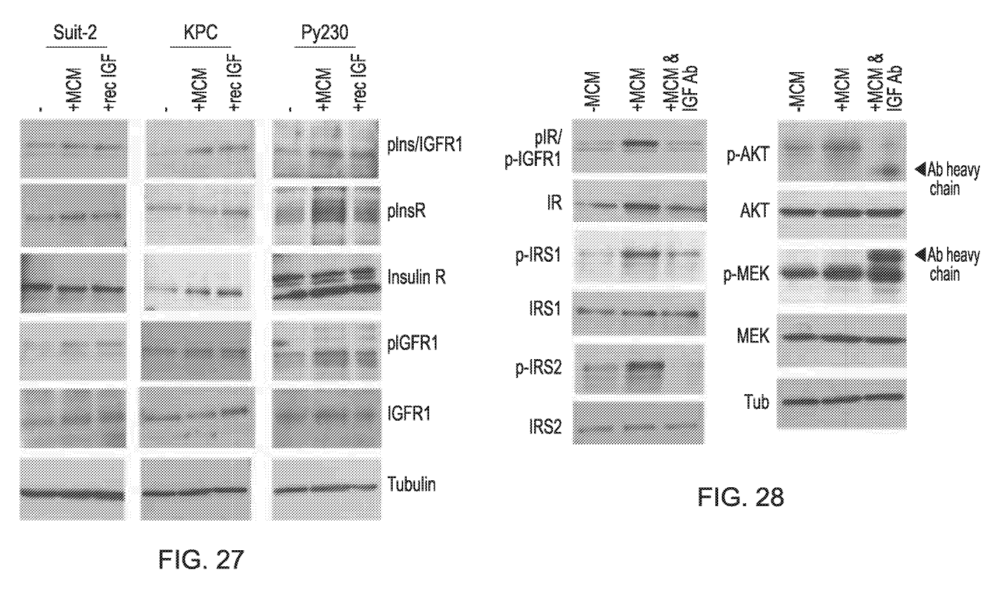

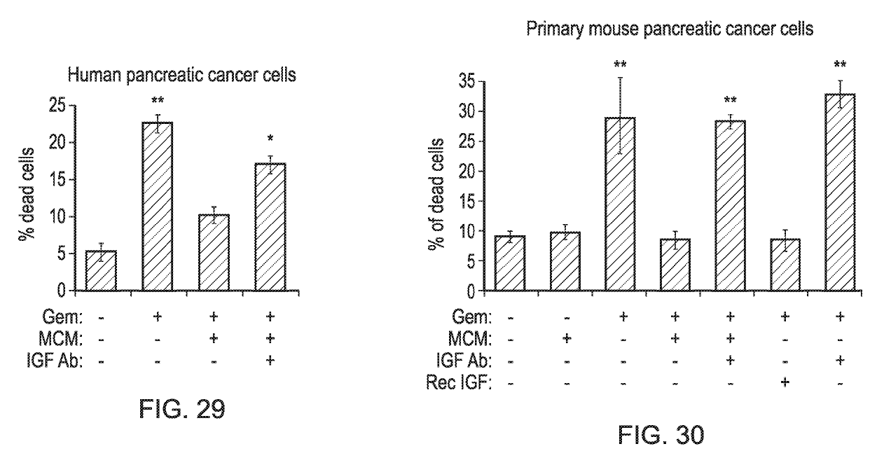

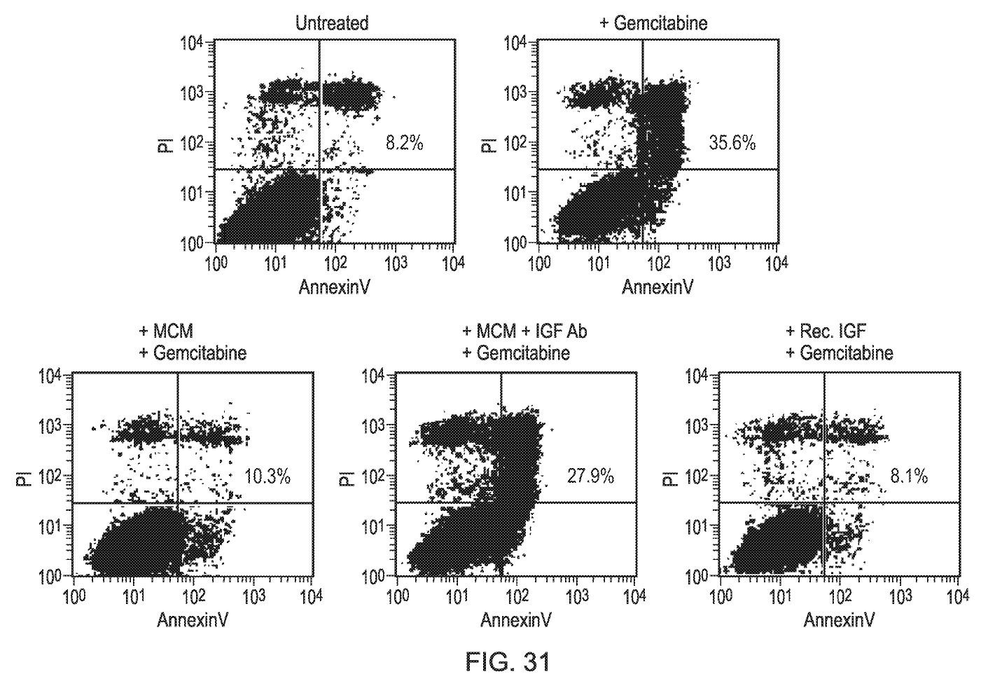

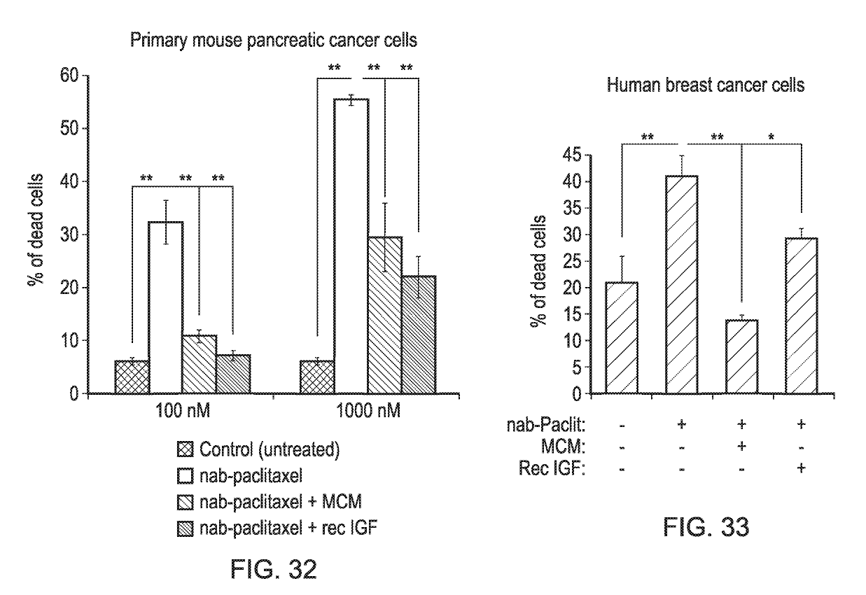

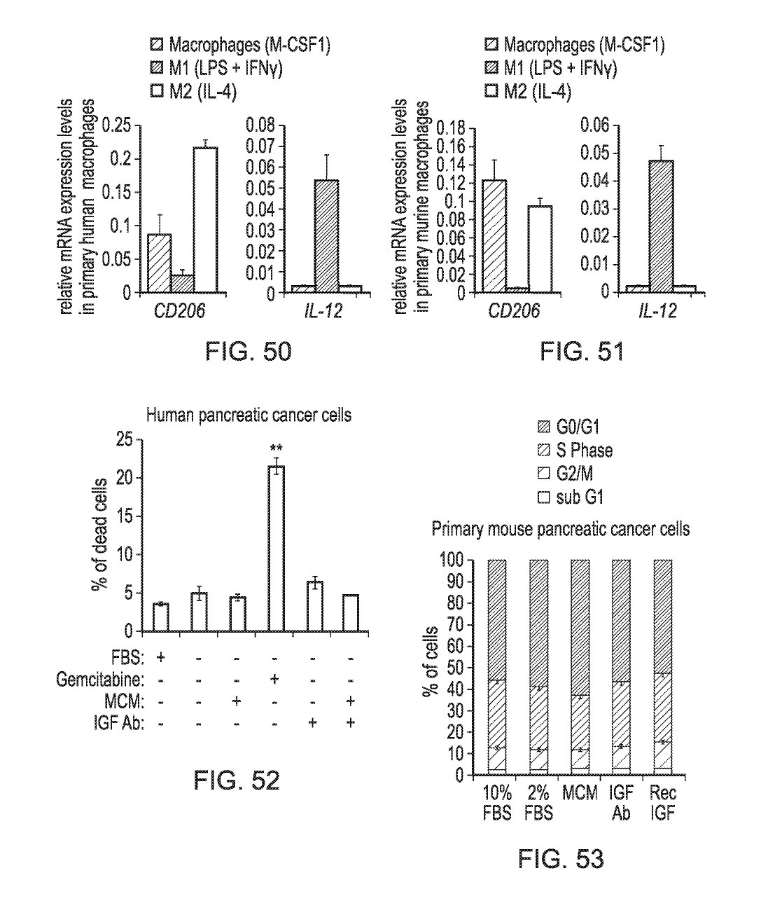

TAMs promote chemoresistance of cancer cells (De Palma and Lewis, 2013; Mantovani and Allavena, 2015; Mielgo and Schmid, 2013; Noy and Pollard, 2014; Ruffell and Coussens, 2015), however the molecular mechanism(s) by which macrophages induce cancer chemoresistance remain elusive. To understand whether macrophages can directly impact on cancer cells' response to chemotherapy, we co-cultured pancreatic and breast cancer cells with primary macrophages or macrophage conditioned media (MCM) in the presence or absence of gemcitabine or paclitaxel, and assessed tumor cell death by flow cytometry. Co-culture of breast and pancreatic cancer cells with primary macrophages or MCM resulted in resistance of cancer cells to chemotherapy (FIG. 20 and FIG. 49). MCM was generated from primary human and mouse macrophages cultured under standard conditionsin the presence of macrophage colony stimulating factor 1 (M-CSF1). We found that,cultured under these standard conditions, macrophages had rather an M2-like phenotype and expressed CD206 but not IL-12 (FIGS. 50 and 51), suggesting that M2-like macrophages are capable of inducing chemoresistance in pancreatic cancer cells (FIG. 20 and FIG. 49). To further confirm this, we polarized macrophages into M1 (with LPS and IFNγ) or M2 macrophages and cultured breast cancer cells with M1 or M2 MCM in the presence or absence of paclitaxel. Interestingly, only MCM from M2-like (but not M1-like) polarized macrophages was capable of inducing resistance of cancer cells to chemotherapy (FIG. 20c). Mechanistically, a phospho-receptor tyrosine kinase array and immunoblotting analysis of pancreatic cancer cells cultured in the presence or absence of MCM revealed that MCM induces the phosphorylation and activation of three receptor tyrosine kinases (RTKs), Insulin/IGF receptor (number 1), AXL receptor (number 2) and Ephrin receptor (number 3) (FIGS. 21 and 22). We only focus on the Insulin receptor because only blockade of the Insulin receptor signaling pathway was found to have an effect on macrophage- mediated chemoresistance. Activation of Insulin and IGF receptors by macrophages was further confirmed by detection of tyrosine phosphorylation on Insulin and IGF receptor immunoprecipitated from Suit-2 pancreatic cancer cells cultured in the presence or absence of MCM (FIG. 23). To understand, how macrophages activate Insulin/IGF receptor signaling in cancer cells, we assessed the expression levels of the three ligands Insulin, IGF-1 and IGF-2 in primary human and mouse macrophages. Interestingly, M2-like (but not M1-like) macrophages express IGF-1 and IGF-2 but not Insulin (FIGS. 24-26). In addition, expression of IGF-1 was significantly increased when macrophages were exposed to tumor conditioned media (TCM) from pancreatic or breast cancer cells, while IGF-2 levels remained generally unchanged, except for the murine macrophages treated with the TCM from Py230 breast cancer cells (FIGS. 24 and 26). Immunoblotting analysis of Suit-2 human pancreatic cancer cells, KPC-derived primary murine pancreatic cancer cells and Py230 primary murine breast cancer cells cultured in the presence or absence of MCM or recombinant IGF, further confirmed that MCM (similarly to recombinant IGF) activates Insulin/IGFR signaling in pancreatic and breast cancer cells (FIG. 27). Importantly, blockade of IGF ligands with an IGF neutralizing antibody was able to prevent activation of Insulin/IGF receptor and its downstream effectors IRS1, IRS2 and AKT by MCM (FIG. 28) and to inhibit macrophage-induced chemoresistance of cancer cells (FIGS. 29-31). In contrast, in the absence of MCM, IGF blockade did not further increase the response of cancer cells to chemotherapy confirming that, at least in this co-cultured system, macrophages (and not tumor cells) are the source of IGF that mediates chemoresistance (FIG. 30). In addition, recombinant IGF was sufficient to mediate resistance of pancreatic and breast cancer cells to gemcitabine and nab-paclitaxel (FIGS. 30-33). Treatment of tumor cells with MCM, IGF blocking antibody or recombinant IGF alone did not alter the survival or proliferation of cancer cells, (FIGS. 52 and 53). Taken together, these results indicae that, while under non-stress conditions, tumor cells do not depend on IGF to survive and proliferate, when challenged with chemotherapy, IGF secreted by macrophages activates the Insulin/IGF receptor signaling pathway, which becomes essential for cancer cells' resistance to chemotherapy.

Insulin/IGF Receptors are Activated on Tumor Cells in Biopsies from Pancreatic and Breast Cancer Patients and this Positively Correlates with Increased M2-like Macrophage Infiltration in Tumors and Tumor Progression.

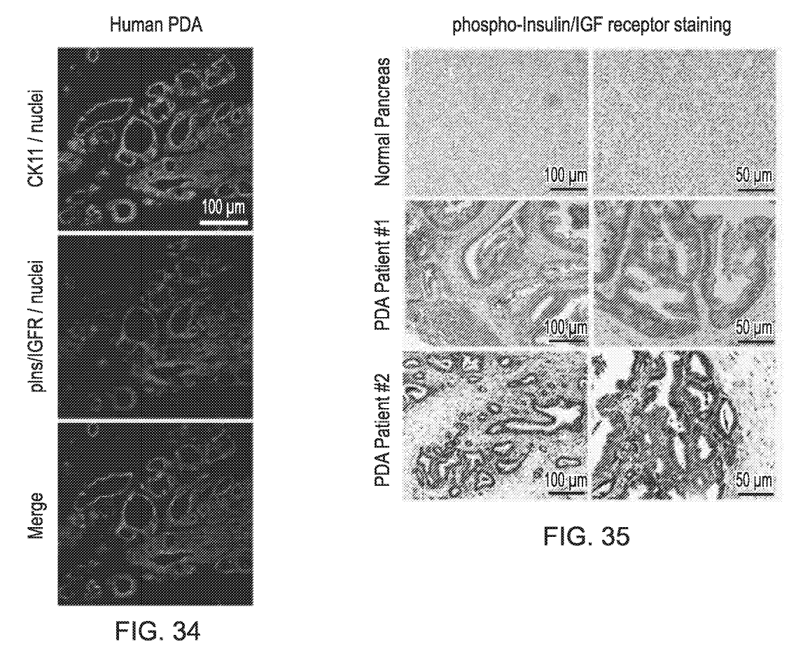

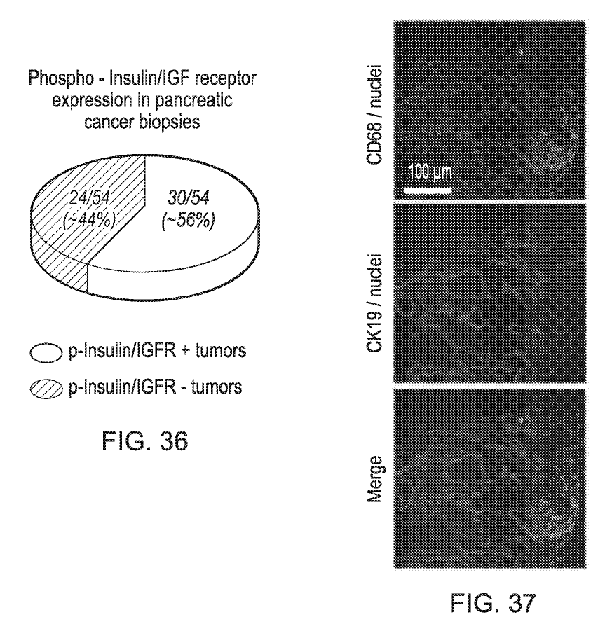

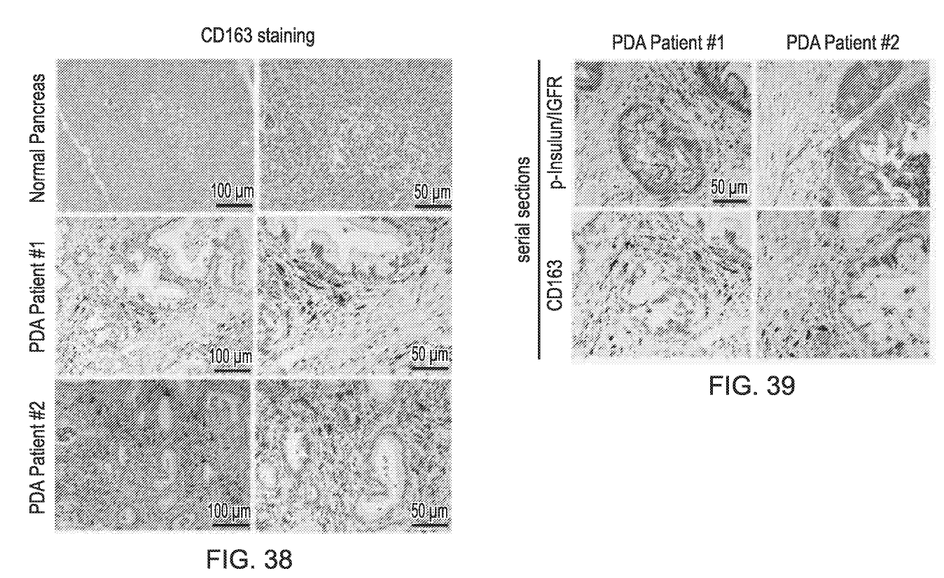

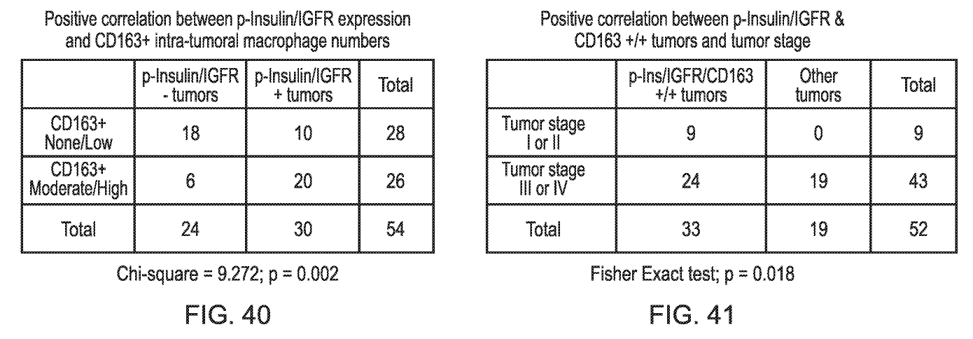

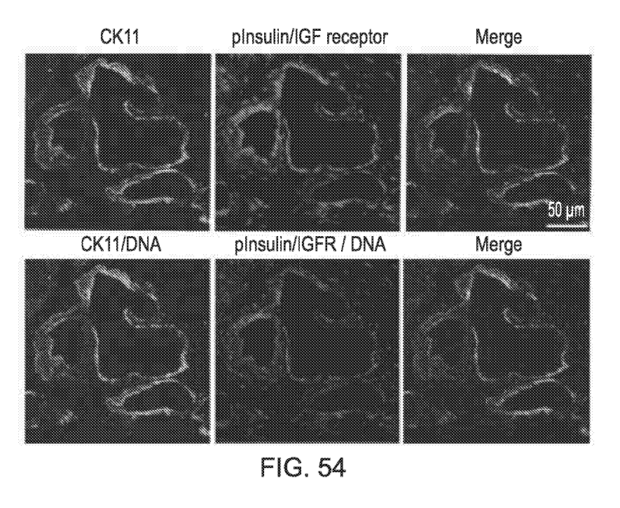

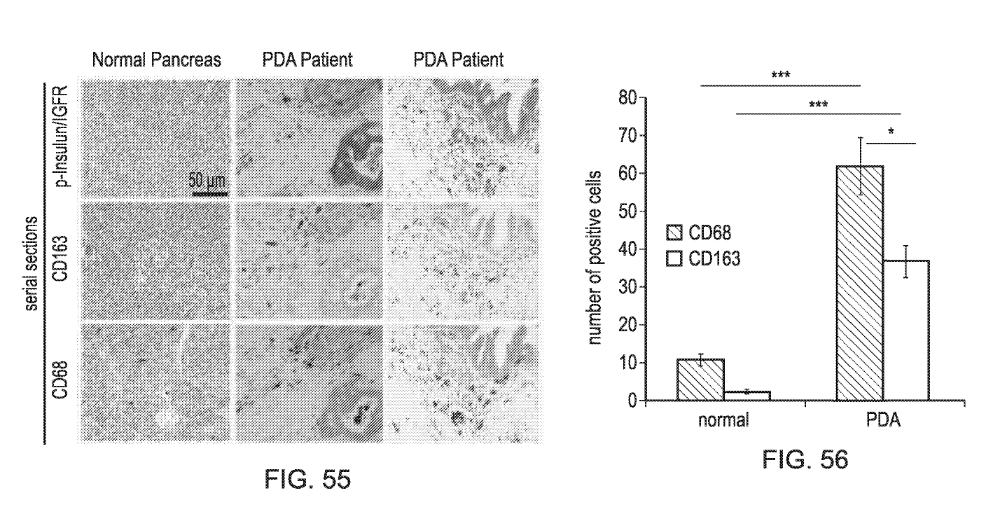

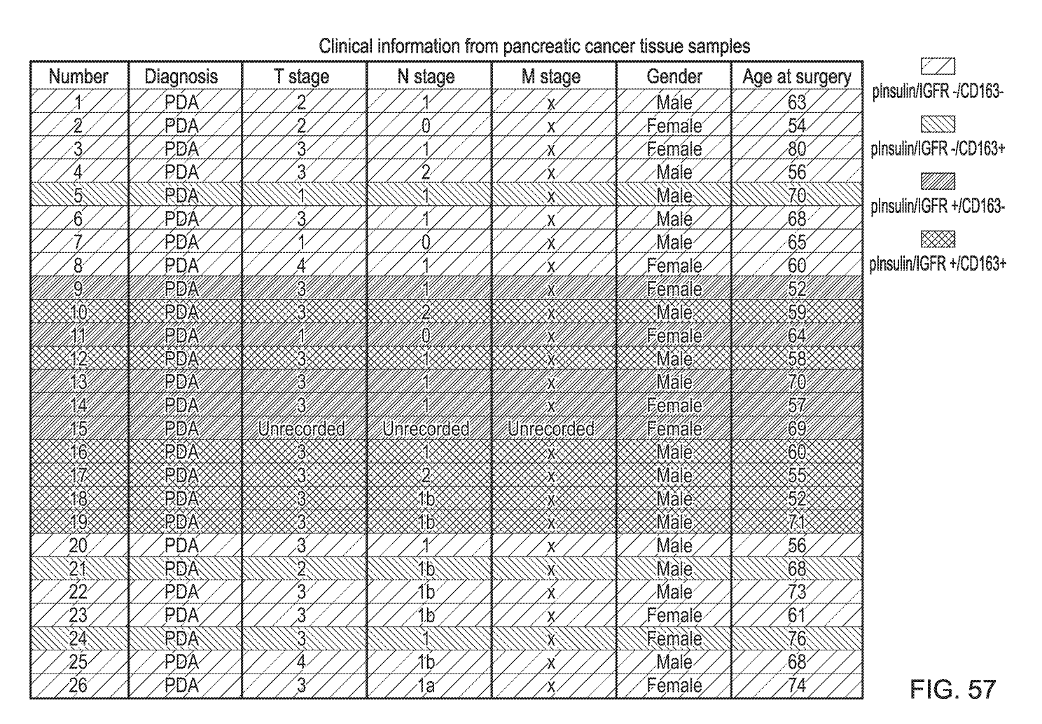

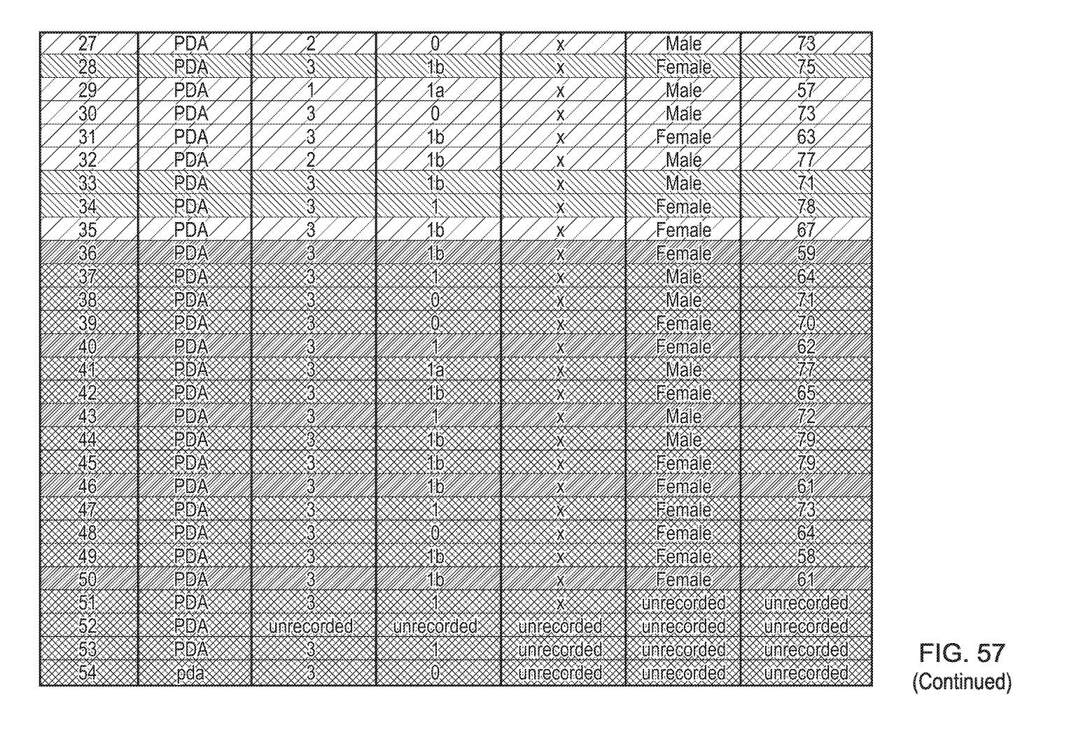

On the basis of these findings, which indicate an important role for M2-like macrophage-derived IGF in activating the insulin/IGF receptor survival signaling pathway in cancer cells, we evaluated whether the Insulin receptor is activated in biopsies from pancreatic cancer patients and whether this correlates with increased macrophage infiltration. Immunofluorescent and Immunohistochemical staining of phopho-lnsulin/IGFR in frozen and paraffin embedded human PDA samples revealed that, indeed Insulin/IGFR was activated on the ductal epithelial pancreatic cancer cells in 30 out of 54 (˜56%) consented patients (FIGS. 34-36 and FIG. 54). Similarly, immunofluorescent and immunohistochemical staining of CD68 and CD163 in the same frozen and paraffin embedded human PDA samples showed that pancreatic tumors are infiltrated by macrophages that surround the ductal epithelial pancreatic cancer cells (FIGS. 37, 38, 55 and 56). Since we previously found that M2-like (but not M1-like) macrophages enhance chemoresistance by activating Insulin/IGFR on the tumor cells, we subsequently analyzed by immunohistochemistry serial sections from the consented 54 PDA patients for the co-presence of M2-like macrophages (CD163+) and phospho-lnsulin/IGFR+ tumor cells. Importantly, we found that activation of Insulin/IGF receptor positively correlates with increased infiltration of M2-like macrophages (Chi-square=9.272; p=0.002) (FIGS. 39, 40 and FIG. 57) and that co-expression of both phospho-lnsulin/IGFR+ tumor cells and CD163+ M2-like macrophages positively correlates with tumor stage (p=0.018) (FIG. 41).

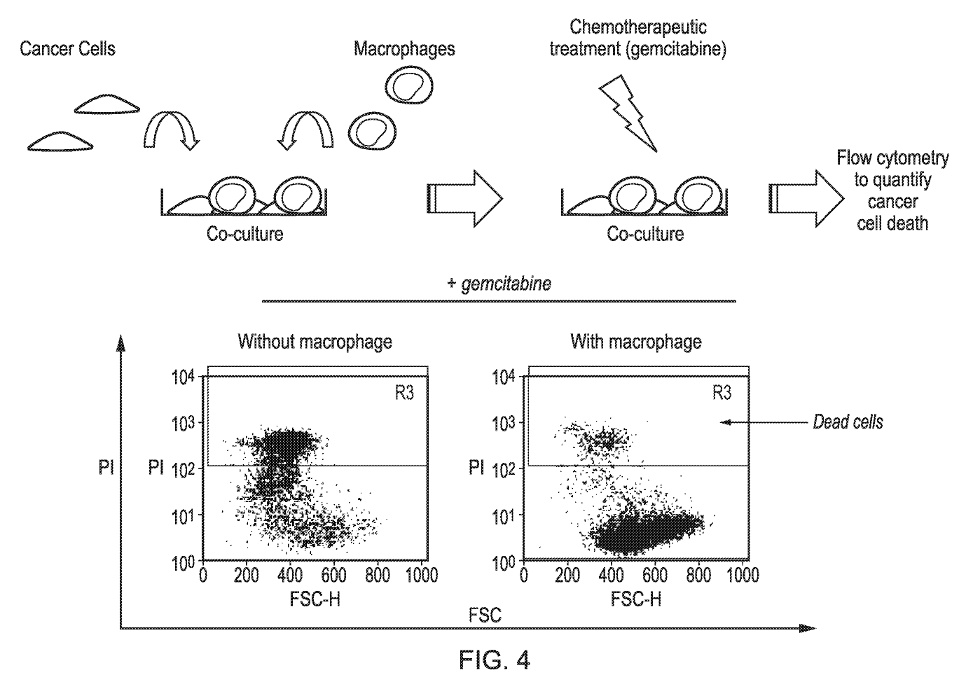

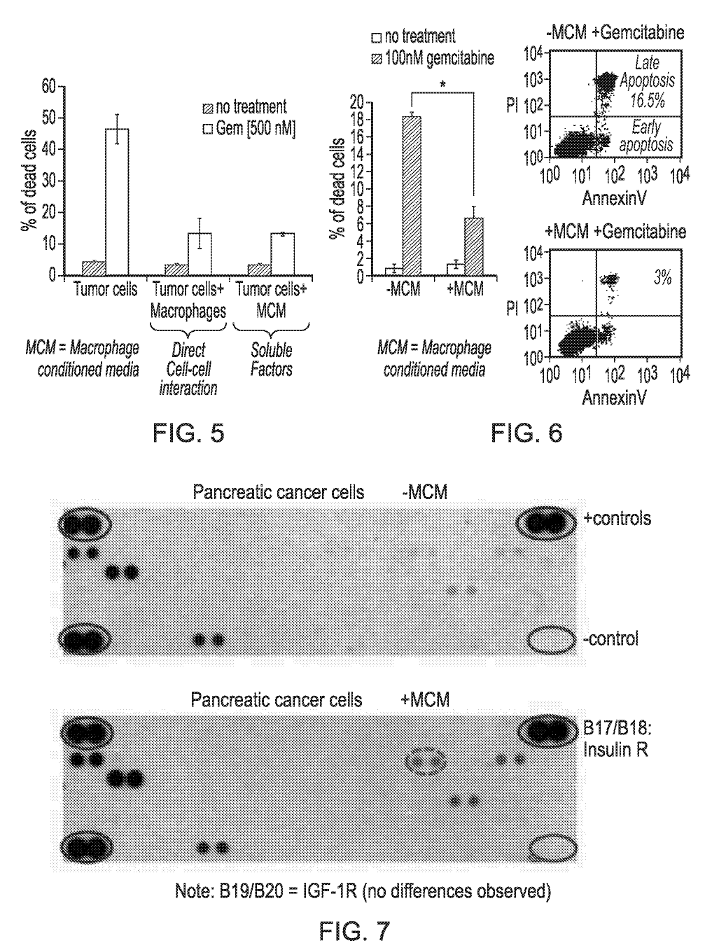

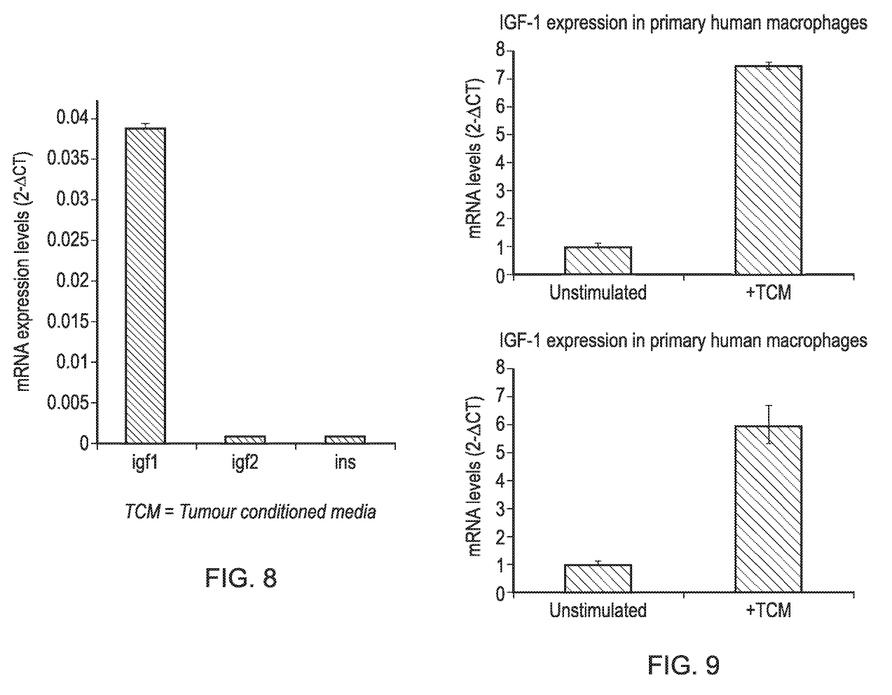

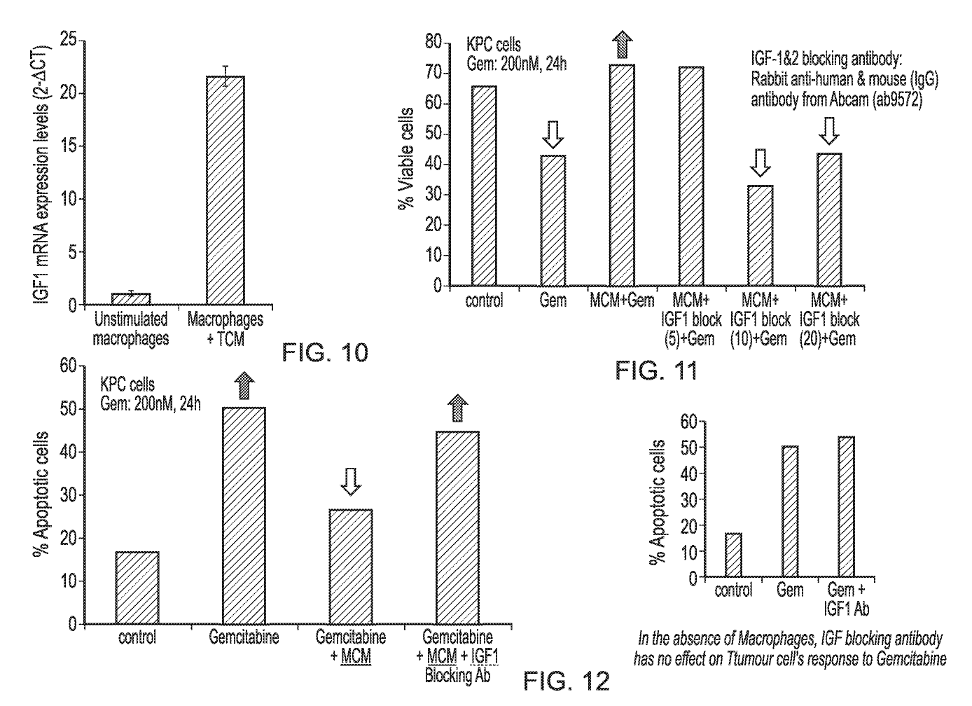

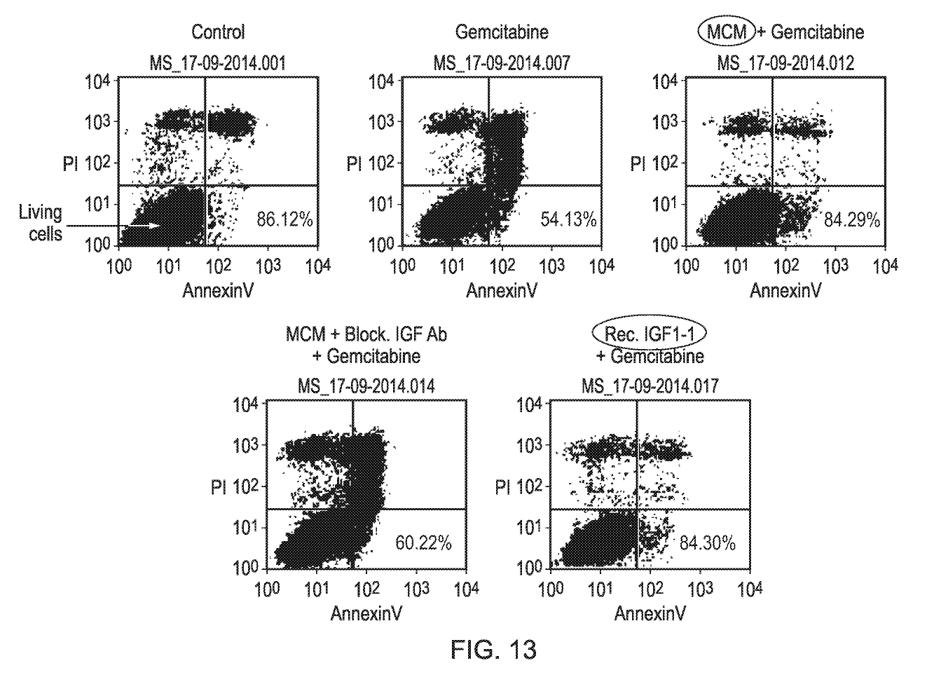

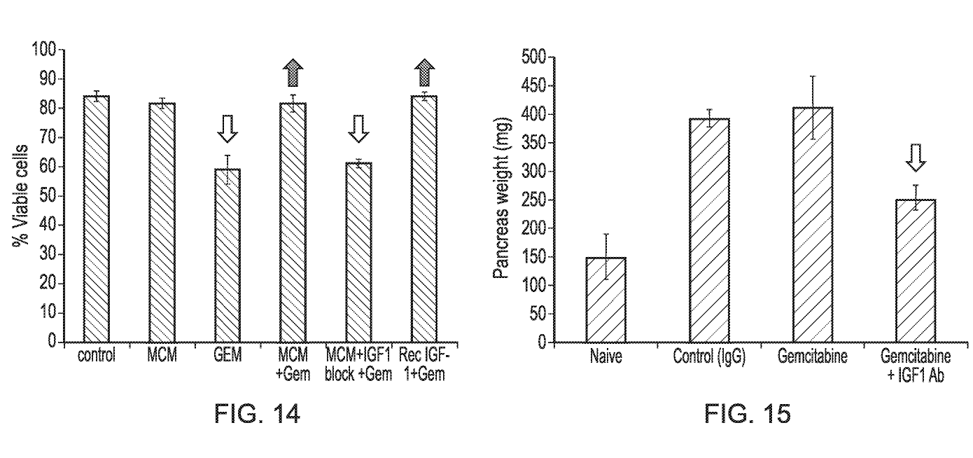

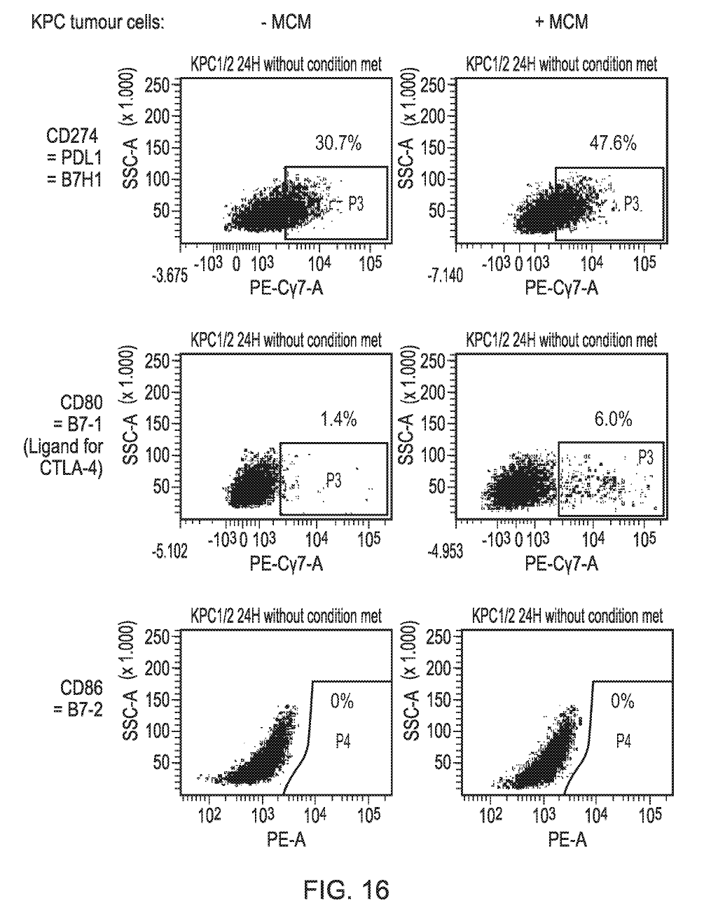

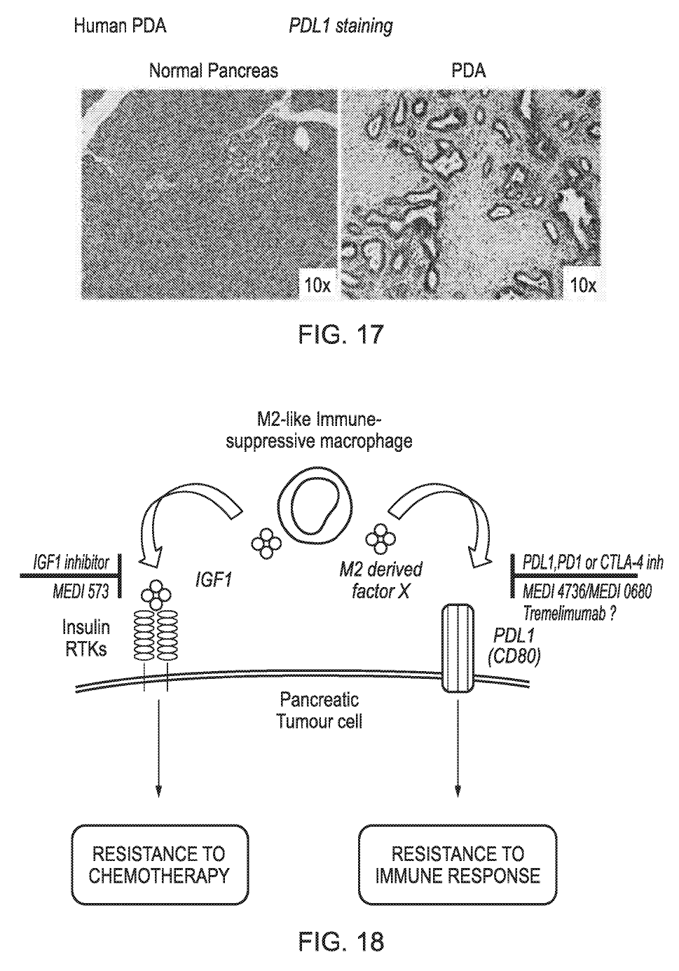

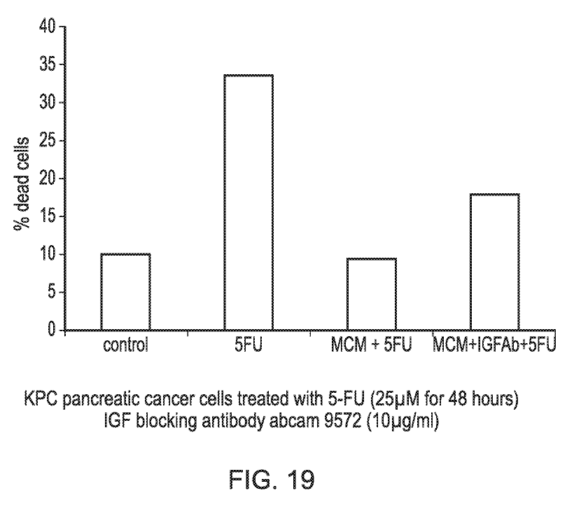

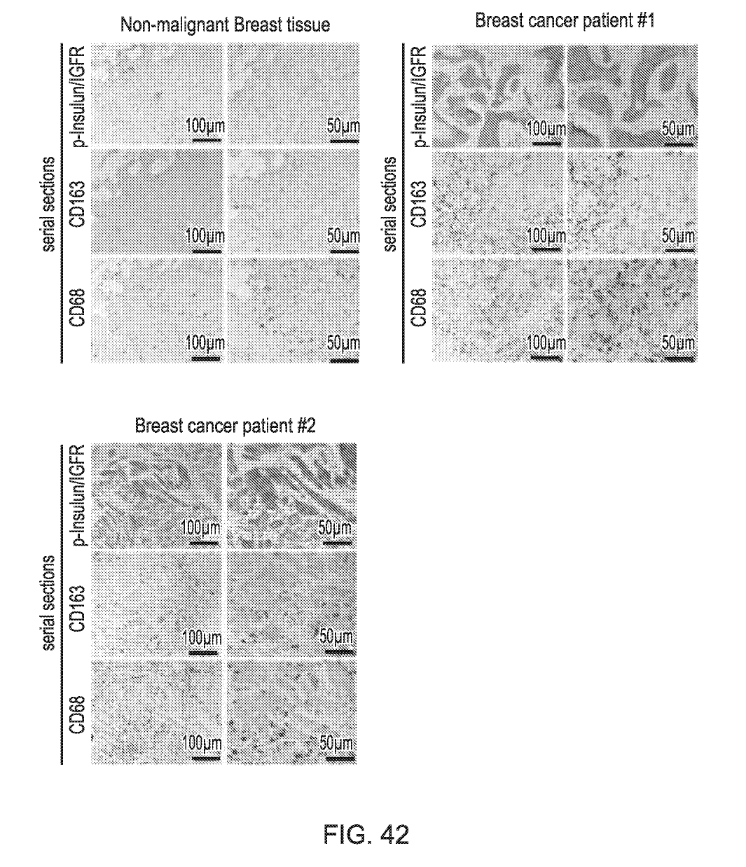

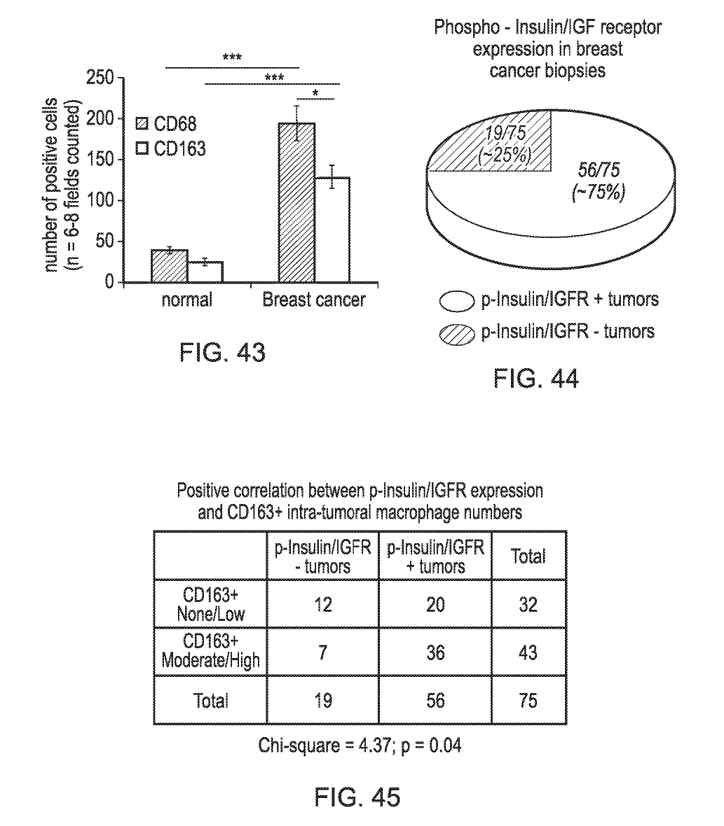

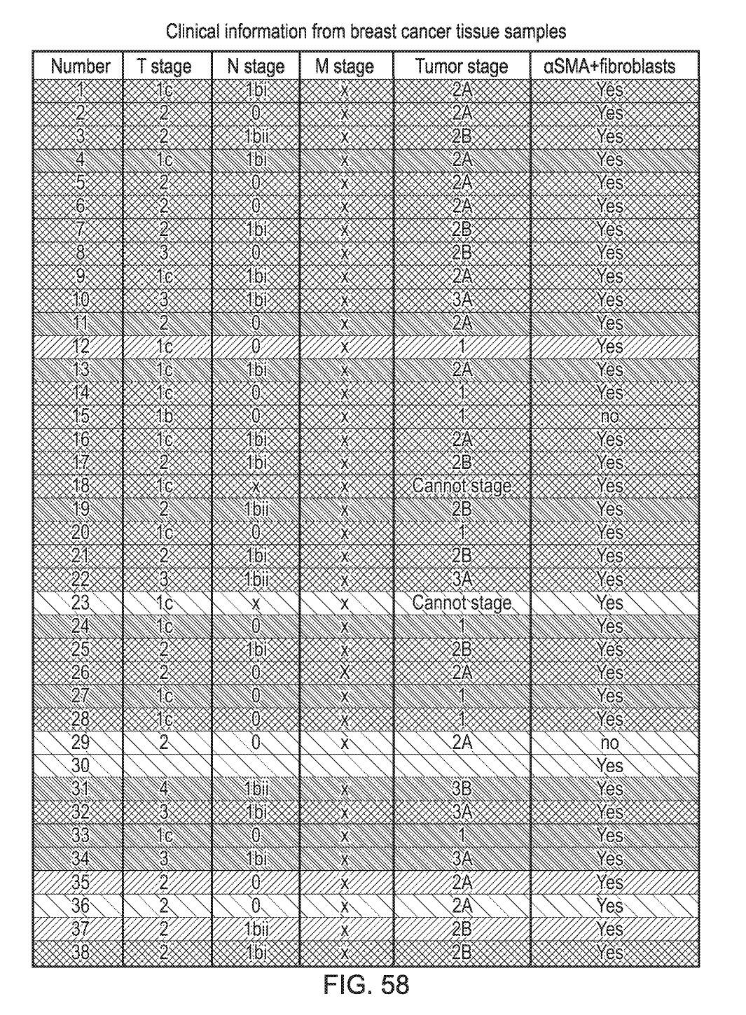

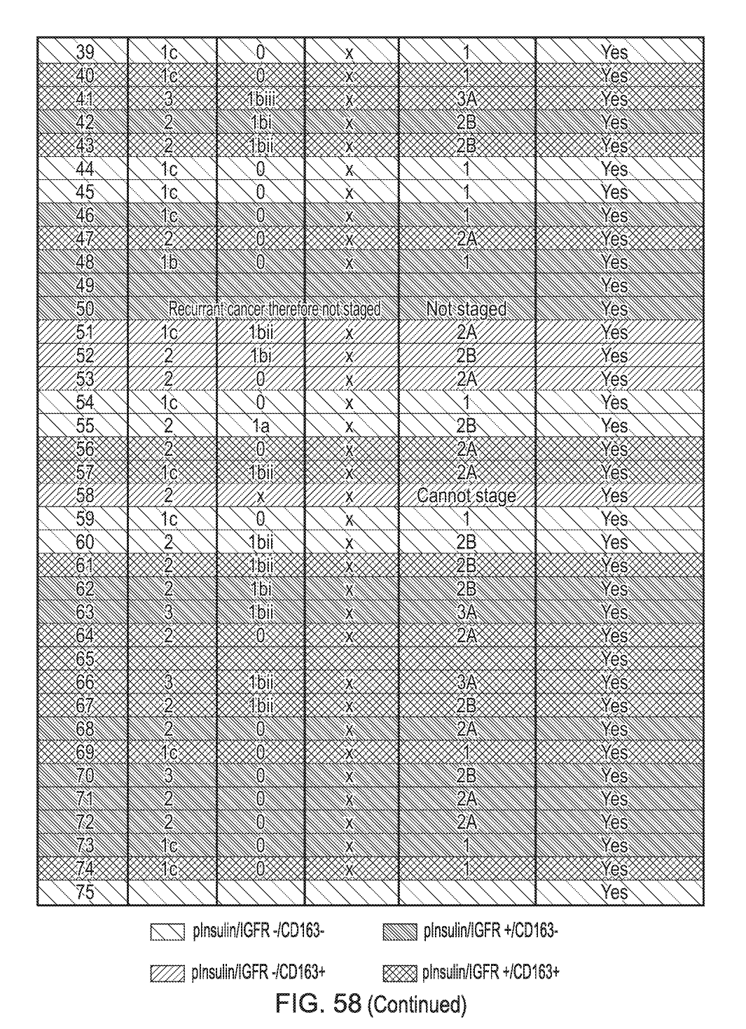

Since we previously found that M2-like macrophages promote chemoresistance of not only pancreatic cancer cells, but also breast cancer cells, by activating the Insulin/IGFR signaling pathway on tumor cells, we analysed serial sections of 75 consented breast cancer patient samples for the co-expression of phospho-lnsulin/IGFR on tumor cells and macrophages (CD68+ and CD163+). Similar to what we observed in PDA patients, we found that breast cancer cells positive for phospho-lnsulin/IGFR were surrounded by macrophages, especially M2-like CD163+macrophages (FIGS. 42 and 43). 56 out of the 75 (˜75%) analysed breast cancer samples were positive for phospho-Insulin/IGFR expression on tumor cells (FIG. 44 and FIG. 58) and activation of the Insulin/IGFR positively correlated with increased M2-like macrophage (CD163+) infiltration (Chi-square=4.37; p=0.04) (FIG. 4f)

Intra-Tumoral M2-like Macrophages and αSMA+ Stromal Cells are the Main Sources of IGF in the Pancreatic Tumor Microenvironment

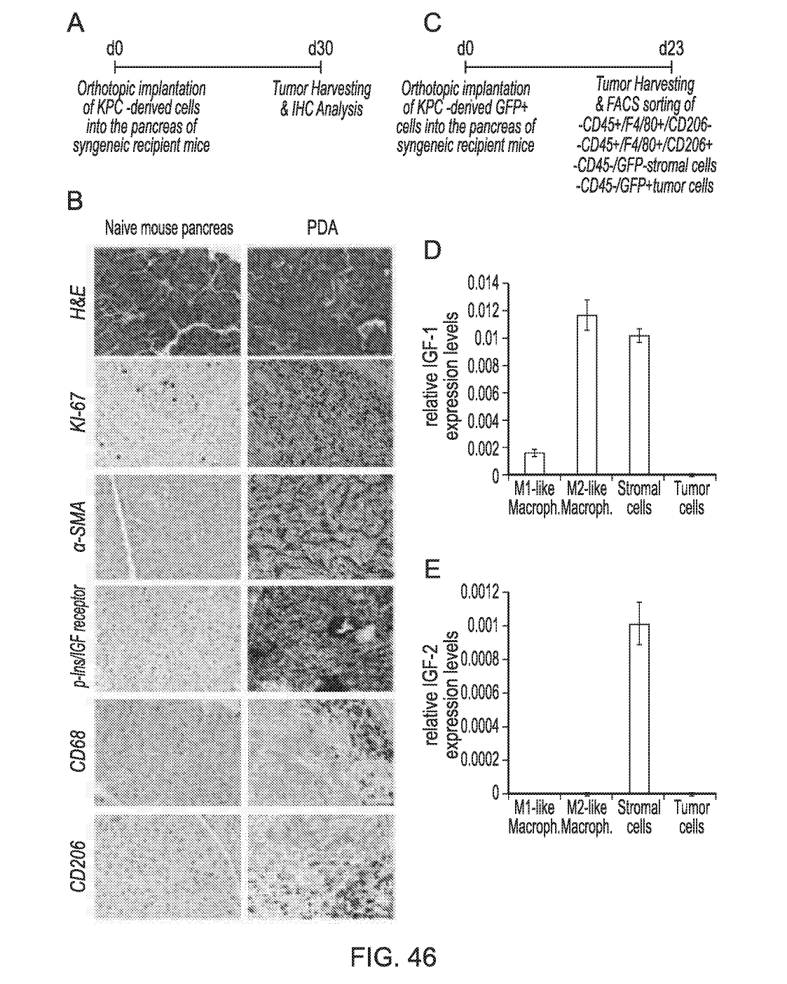

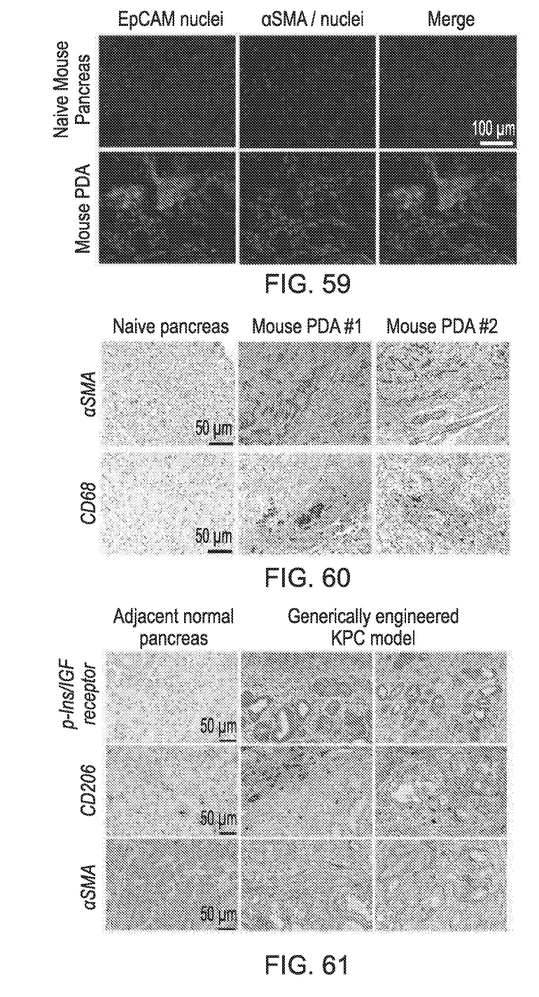

Primary murine PDA cells were isolated from a genetically engineered mouse model of pancreatic cancer Kras^{G12D};Trp53^{R172H};Pdx1-Cre (KPC) mice. KPC-derived cells were maintained on collagen coated plates at a minimal passage number (<p8) to allow initial expansion. KPC cells were then orthotopically implanted into isogenic immune-competent recipient mice (FIG. 46a). H&E and immunohistochemical staining of these tumors with the proliferation marker Ki-67 confirmed that these mice developed PDA (FIG. 46b). Similar to human PDA and to the genetically engineered KPC mouse model, these tumors are rich in αSMA+ myofibroblasts, are infiltrated by CD68+ macrophages and CD206+ M2-like macrophages and show activation of Insulin/IGFR (FIG. 46b and FIGS. 59-62).

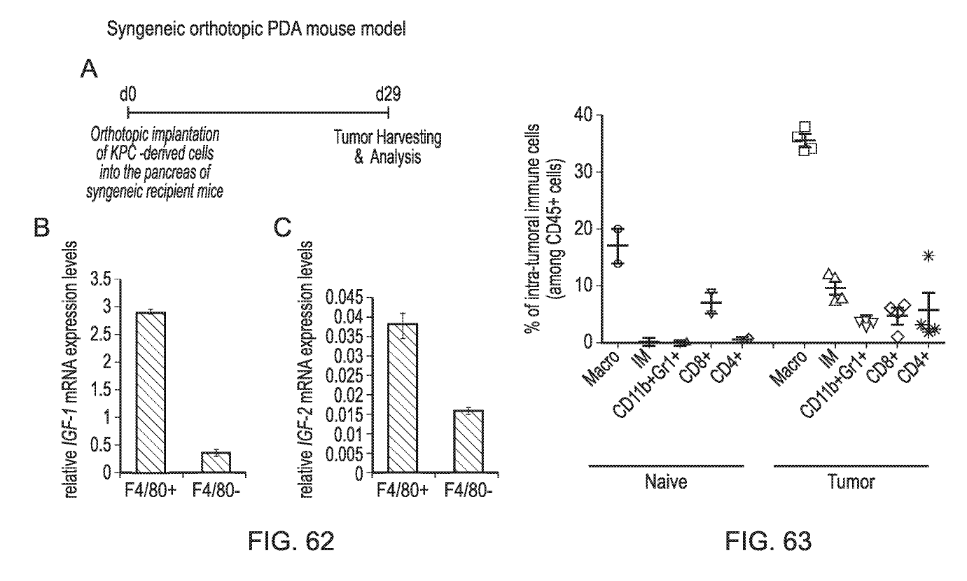

To determine whether in vivo TAMs express IGFs, we first isolated TAMs (F4/80+cells) from established KPC tumors and assessed the expression levels of Insulin, IGF-1 and IGF-2. In agreement with our previous in vitro findings (FIG. 20), we found that intra-tumoral TAMs isolated from murine pancreatic tumors express IGF-1 and IGF-2 and are a major source of these growth factors within the tumor microenvironment, expressing 3-10 times more IGF ligands compared to other (F4/80−) cells within the tumor microenvironment (FIG. 62b, c).

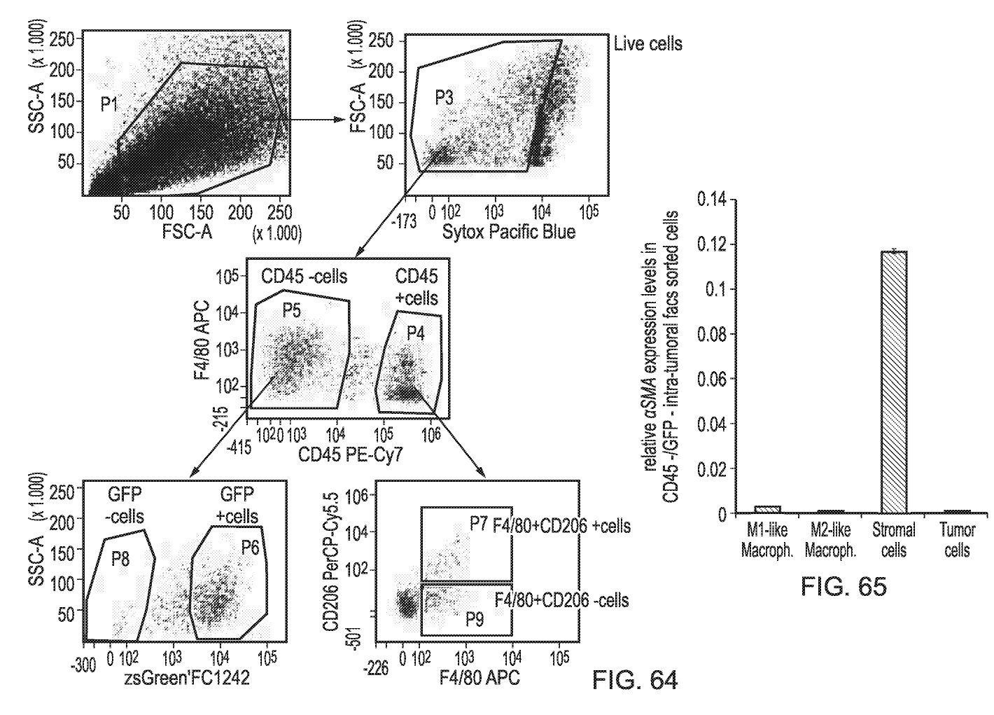

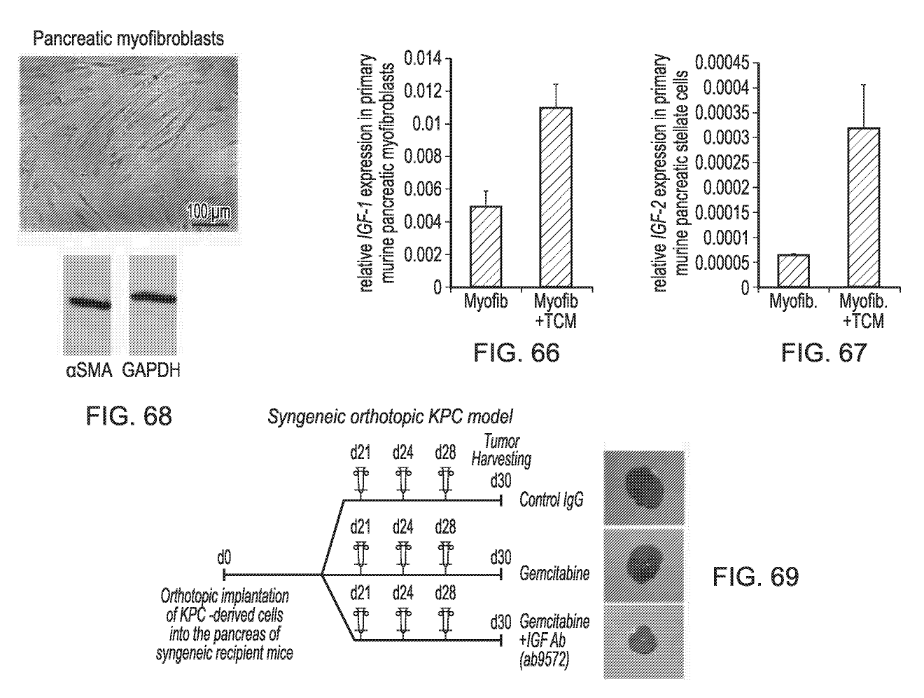

To further investigate which cell population(s) within the tumor are the main producers of IGF, we used an alternative mouse model in which we orthotopically implanted into the pancreas of syngeneic recipient mice KPC-derived GFP+ cells (FIG. 46c). Tumors were harvested at day 23 and CD45−/GFP+ tumor cells, CD45−/GFP− non-immune stromal cells, CD45+/F4/80+/CD206− M1-like macrophages and CD45+/F4/80+/CD206+M2-like macrophages were sorted by flow cytometry (FIG. 64) and analysed for the expression of IGF1 and IGF2 (FIG. 46c,d). This model further confirmed that M2-like (but not M1-like) macrophages express IGF, but interestingly, also revealed non-immune αSMA+ stromal cells (also known as myofibroblasts) as an additional source of IGF production (FIG. 46d, e and FIG. 65). To further confirm myofibroblasts as an additional source of IGF production, we isolated primary pancreatic stellate cells from naïve mice pancreas. Pancreatic stellate cells when activated become myofibroblasts and express αSMA (FIG. 66). We found that primary pancreatic myofibroblasts expressed IGF-1 and 2 and the expression of both IGF ligands further increased when myofibroblasts were exposed to KPC-derived TCM (FIGS. 67-68).

Blockade of IGFs Improves Response to Chemotherapy in a Pre-Clinical Tumor Model of Pancreatic Cancer

αSMA+ stromal cells together with macrophages are the most abundant non-malignant stromal cells within the tumor microenvironment. The fact that these two stromal cell types are the main sources of IGF in the tumor microenvironment and that IGF signaling protects tumor cells from response to chemotherapy suggests that inhibiting IGF, in vivo, could increase tumor response to chemotherapy.

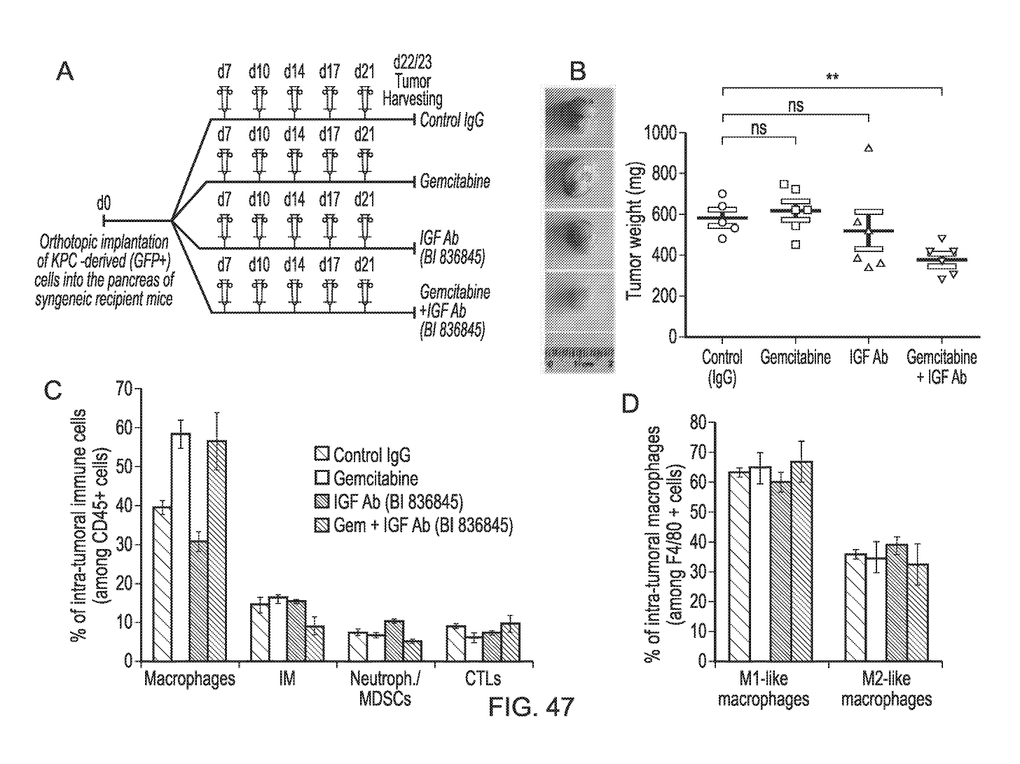

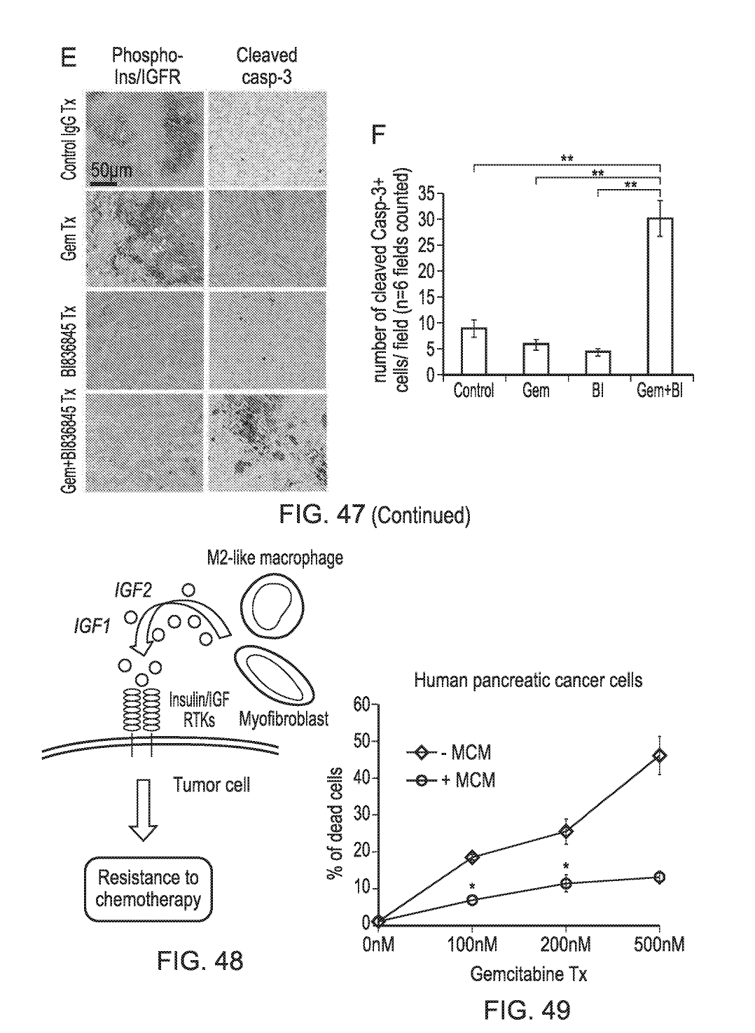

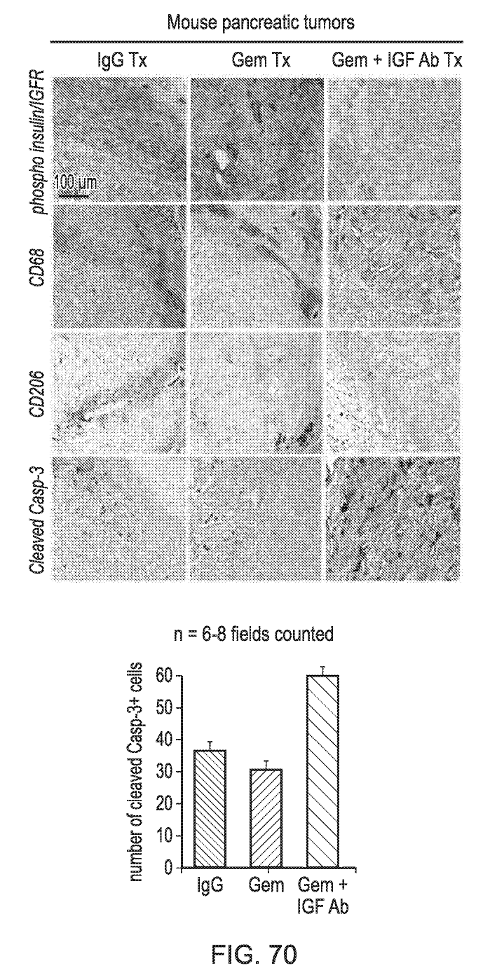

To test this hypothesis, we treated mice bearing established orthotopic pancreatic tumors with control IgG antibody, gemcitabine alone, IGF blocking antibody (BI 836845) alone or gemcitabine with IGF blocking antibody. Treatments were administered intraperitoneally, twice a week for two weeks (FIG. 47a). As previously shown (Mitchem et al., 2013; Torres et al., 2013; Zhu et al., 2014) and similar to what is observed in PDA patients, gemcitabine alone had no effect on tumor growth (FIG. 47b). Treatment with IGF blocking antibody alone only show a modest effect on tumor growth. In contrast, combination of gemcitabine with IGF blocking antibody significantly reduced tumor growth (FIG. 47b). Analysis of immune cells populations within the different treatment groups showed an increase in F4/80+ macrophages in gemcitabine and gemcitabine+IGF antibody treated tumors (FIG. 47c), however the ratio of M1-like (CD206-) and M2-like (CD206+) macrophages within tumors remained the same in all treatment groups with approximately ⅓ of the intra-tumoral macrophages being M1-like macrophages and ⅔ being M2-like macrophages (FIG. 47d). These findings suggest that while gemcitabine seems to trigger an increase in macrophage infiltration, none of the treatments seems to alter macrophage polarization. In addition, the percentage of inflammatory monocytes (Ly6C+/Ly6G−/CD11b+), Neutrophils/Myeloid derived suppressor cells (Gr1+, CD11b+) and cytotoxic CD8+ T cells (CTLs) remained the same in all treatment groups (FIG. 47c). Immunohistochemical analysis of tumors, showed that phospho-lnsulin/IGFR expression was decreased in tumors treated with IGF blocking antibody. Importantly, immunohistochemical staining of cleaved caspase-3 revealed higher levels of cell death in tumors treated with the combination of gemcitabine and the IGF blocking antibody, compared to those treated with an IgG irrelevant antibody, gemcitabine or IGF antibody alone. A similar in vivo experiment, using a different KPC-derived cell line and a different IGF blocking antibody (ab9572) yielded very similar results (FIGS. 69, 70).

Discussion

The data presented herein describe, for the first time, intra-tumoral stromal-derived IGF as a critical inducer of chemoresistance in pancreatic and breast cancer and provide pre-clinical data that support the rational of using IGF neutralizing antibodies with chemotherapy for the treatment of pancreatic cancer (FIG. 48). We report a direct role for M2-like macrophages on chemoresistance of pancreatic and breast cancer cells and a paracrine signaling loop in which macrophage-derived IGF activates the Insulin/IGFR survival signalling pathway and blunts response of pancreatic and breast cancer cells to gemcitabine and paclitaxel. While IGFR inhibitors failed in the clinics (Guha, 2013; Pollak, 2012), two IGF blocking antibodies, MEDI-573 and BI836845, are currently being evaluated in phase 2 and phase 1 clinical trials for metastatic breast cancer and various solid cancers, respectively. Our mouse tumor studies described here provide the proof-of-principle for the use of IGF-blocking antibodies in combination with chemotherapeutic agents such as gemcitabine for the treatment of patients with pancreatic cancer, and suggest that inhibiting IGF ligands, and thereby preventing signaling through both Insulin and IGF receptors, may be a better therapeutic strategy. In addition, the fact that IGF is also able to increase resistance of breast and pancreatic cancer cells to paclitaxel and nab-paclitaxel, suggests that IGFs blockade may also increase efficacy of other cytotoxic agents in a variety of cancer types associated with a dominant presence of TAMs. In agreement with this, autocrine IGF1-signaling was recently found to mediate chemoresistance in melanoma (Obenauf et al., 2015).

In addition, we found that in humans, the Insulin/IGFR signaling pathway is activated in 57% and 75% of pancreatic and breast cancer patient samples respectively, and this correlates with a rich stromal compartment with increased M2-like macrophage infiltration and with advanced tumor stage. These observations suggest that combining chemotherapy with IGF blockade could be beneficial for patients in which the Insulin/IGF receptor is activated, and that Insulin/IGFR activation together with increased M2-like macrophage infiltration could be used in the future as a predictive biomarker for response to therapy enabling patient stratification for treatment with anti-IGF therapy in combination with chemotherapy.

Despite extensive efforts invested in the clinical development of therapies against PDA, current standard treatments only exert a modest efficacy and targeting only the tumor cells has not resulted in a significant improvement of PDA treatment. Similarly, triple negative breast cancer has a very poor survival rate and better strategic therapies are urgently needed. The rich stromal compartment present in both PDA and breast cancer is not just a bystander but is a source of a variety of non-malignant cells and extracellular matrix proteins, which support tumor progression, resistance to cytotoxic agents and metastasis. Thus, therapies that target both the neoplastic cells and the tumor microenvironment, will achieve better therapeutic response (Bailey and Leach, 2012; Costello et al., 2012; Feig et al., 2012; Hidalgo et al., 2015; Paulson et al., 2013; Werner et al., 2013). Macrophages and myofibroblasts are the two main stromal cell types within the tumor microenvironment in solid cancers. Interestingly, we found that in tumors, both M2-like macrophages and myofibroblasts are the main sources of IGF production and that inhibiting IGF increased the response of tumor cells to chemotherapy in a pre-clinical model of PDA. Thus, these studies provide a new combinatorial therapeutic opportunity to treat these devastating diseases.

TAMs can enhance or limit the efficacy of chemotherapy depending on the tumor model and/or the chemotherapeutic agent used (De Palma and Lewis, 2013; Mantovani and Allavena, 2015; Mielgo and Schmid, 2013; Noy and Pollard, 2014; Ruffell and Coussens, 2015). In fact, chemoresistance is increased when cytotoxic agents increase M2-like macrophage infiltration via CCL2 (Nakasone et al., 2012) or CSF1 (DeNardo et al., 2011). Macrophages can also impair host responses to chemotherapy by expressing cathepsins that activate chemoprotective T cells (Bruchard et al., 2013; Shree et al., 2011) or by inducing the upregulation of cytidine deaminase, an enzyme that metabolizes nucleoside analogs (Weizman et al., 2014). TAMs demonstrate a high degree of plasticity, and can be polarized into M1-like anti-tumorigenic and M2-like pro-tumorigenic macrophages. Previous studies targeting signaling pathways necessary for the recruitment of macrophages or specific chemotactic factors (CCL2/PI3K⊐/CSF1) have provided proof of concept that macrophages represent an attractive target to reduce tumor growth (DeNardo et al., 2011; Mitchem et al., 2013; Pyonteck et al., 2013; Ruffell and Coussens, 2015; Schmid et al.,201 la; Schmid et al., 2011b; Schmid et al., 2013; Zhu et al., 2014). However, dependent on the microenvironmental cytokine milieu, macrophages are not only promoting tumor growth, but they can also critically orchestrate an anti-tumor immune response (Mantovani and Allavena, 2015). Thus, therapies that aim to specifically inhibit the pro-tumorigenic functions of macrophages while sparing their tumoricidal activity could act as an alternative, and perhaps, more efficient approach than therapies that completely block macrophage recruitment to the tumor (Bronte and Murray, 2015; Quail and Joyce, 2013). In this regard, our in vivo studies indicate that IGF blockade induces tumors' response to gemcitabine without affecting immune cell infiltration, including macrophage infiltration or macrophage polarization. In conclusion, our studies suggest that M2-like macrophages and myofibroblasts can blunt host responses to chemotherapy via a IGFs-Insulin/IGFR paracrine signaling axis, and that IGF blockade improves gemcitabine efficacy in a pancreatic cancer model providing the rationale for moving this type of combinatorial treatment forward in the clinic.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### REFERENCES

1. Basu, B., Olmos, D., and de Bono, J. S. (2011). Targeting IGF-1R: throwing out the baby with the bathwater? British journal of cancer 104, 1-3.
2. Gore, J., and Korc, M. (2014). Pancreatic cancer stroma: friend or foe? Cancer cell 25, 711-712.
3. Guha, M. (2013). Anticancer IGF1R classes take more knocks. Nature reviews Drug discovery 12, 250.
4. Ino, Y., Yamazaki-ltoh, R., Shimada, K., Iwasaki, M., Kosuge, T., Kanai, Y., and Hiraoka, N. (2013). Immune cell infiltration as an indicator of the immune microenvironment of pancreatic cancer. British journal of cancer 108, 914-923.
5. Kurahara, H., Shinchi, H., Mataki, Y., Maemura, K., Noma, H., Kubo, F., Sakoda, M., Ueno, S., Natsugoe, S., and Takao, S. (2011). Significance of M2-polarized tumor-associated macrophage in pancreatic cancer. The Journal of surgical research 167, e211-219.
6. Li, D., Xie, K., Wolff, R., and Abbruzzese, J. L. (2004). Pancreatic cancer. The Lancet 363, 1049-1057.
7. Lunardi, S., Muschel, R. J., and Brunner, T. B. (2014). The stromal compartments in pancreatic cancer: are there any therapeutic targets? Cancer letters 343, 147-155.
8. McMillin, D. W., Negri, J. M., and Mitsiades, C. S. (2013). The role of tumour-stromal interactions in modifying drug response: challenges and opportunities. Nature reviews Drug discovery 12, 217-228.
9. Mielgo, A., and Schmid, M. C. (2013). Impact of tumour associated macrophages in pancreatic cancer. BMB reports 46, 131-138.
10. Noy, R., and Pollard, J. W. (2014). Tumor-associated macrophages: from mechanisms to therapy. Immunity 41, 49-61.
11. Pollak, M. (2012). The insulin and insulin-like growth factor receptor family in neoplasia: an update. Nature reviews Cancer 12, 159-169.
12. Ruffell, B., Affara, N. I., and Coussens, L. M. (2012). Differential macrophage programming in the tumor microenvironment. Trends in immunology 33, 119-126.
13. Schmid, M. C., Avraamides, C. J., Dippold, H. C., Franco, I., Foubert, P., Ellies, L. G., Acevedo, L. M., Manglicmot, J. R., Song, X., Wrasidlo, W., et al. (2011a). Receptor tyrosine kinases and TLR/IL1Rs unexpectedly activate myeloid cell PI3kgamma, a single convergent point promoting tumor inflammation and progression. Cancer cell 19, 715-727.
14. Schmid, M. C., Franco, I., Kang, S. W., Hirsch, E., Quilliam, L. A., and Varner, J. A. (2013). PI3-kinase gamma promotes Rap1a-mediated activation of myeloid cell integrin alpha4beta1, leading to tumor inflammation and growth. PloS one 8, e60226.
15. Zahreddine, H., and Borden, K. L. (2013). Mechanisms and insights into drug resistance in cancer. Frontiers in pharmacology 4, 28.
16. Myets and Whittaker (2002). Structural Biology of Insulin and IGF1 receptors: Implications for Drug design. Nature reviews drug discovery Vol 1, October 2002, 769-783.
17. Bailey, J. M., and Leach, S. D. (2012). Signaling pathways mediating epithelial-mesenchymal crosstalk in pancreatic cancer: Hedgehog, Notch and TGFbeta. In Pancreatic Cancer and Tumor Microenvironment, P. J. Grippo, and H. G. Munshi, eds. (Trivandrum (India)).
18. Biswas, T., Gu, X., Yang, J., Ellies, L. G., and Sun, L. Z. (2014). Attenuation of TGF-beta signaling supports tumor progression of a mesenchymal-like mammary tumor cell line in a syngeneic murine model. Cancer Lett 346, 129-138.

19. Bronte, V., and Murray, P. J. (2015). Understanding local macrophage phenotypes in disease: modulating macrophage function to treat cancer. Nat Med 21, 117-119. Bruchard, M., Mignot, G., Derangere, V., Chalmin, F., Chevriaux, A., Vegran, F., Boireau,
20. W., Simon, B., Ryffel, B., Connat, J. L., et al. (2013). Chemotherapy-triggered cathepsin B release in myeloid-derived suppressor cells activates the Nlrp3 inflammasome and promotes tumor growth. Nat Med 19, 57-64.
21. Costello, E., Greenhalf, W., and Neoptolemos, J. P. (2012). New biomarkers and targets in pancreatic cancer and their application to treatment. Nat Rev Gastroenterol Hepatol 9, 435-444.
22. De Palma, M., and Lewis, C. E. (2013). Macrophage regulation of tumor responses to anticancer therapies. Cancer Cell 23, 277-286.
23. DeNardo, D. G., Brennan, D. J., Rexhepaj, E., Ruffell, B., Shiao, S. L., Madden, S. F., Gallagher, W. M., Wadhwani, N., Keil, S. D., Junaid, S. A., et al. (2011). Leukocyte complexity predicts breast cancer survival and functionally regulates response to chemotherapy. Cancer Discov 1, 54-67.
24. Feig, C., Gopinathan, A., Neesse, A., Chan, D. S., Cook, N., and Tuveson, D. A. (2012). The pancreas cancer microenvironment. Clin Cancer Res 18, 4266-4276.
25. Gibby, K., You, W. K., Kadoya, K., Helgadottir, H., Young, L. J., Ellies, L. G., Chang, Y., Cardiff, R. D., and Stallcup, W. B. (2012). Early vascular deficits are correlated with delayed mammary tumorigenesis in the MMTV-PyMT transgenic mouse following genetic ablation of the NG2 proteoglycan. Breast Cancer Res 14, R67.
26. Guha, M. (2013). Anticancer IGF1R classes take more knocks. Nat Rev Drug Discov 12, 250.
27. Hidalgo, M., Cascinu, S., Kleeff, J., Labianca, R., Lohr, J. M., Neoptolemos, J., Real, F. X., Van Laethem, J. L., and Heinemann, V. (2015). Addressing the challenges of pancreatic cancer: future directions for improving outcomes. Pancreatology 15, 8-18.

28. Holohan, C., Van Schaeybroeck, S., Longley, D. B., and Johnston, P. G. (2013). Cancer drug resistance: an evolving paradigm. Nat Rev Cancer 13, 714-726.
29. Junttila, M. R., and de Sauvage, F. J. (2013). Influence of tumour micro-environment heterogeneity on therapeutic response. Nature 501, 346-354.
30. Mantovani, A., and Allavena, P. (2015). The interaction of anticancer therapies with tumor-associated macrophages. J Exp Med 212, 435-445.
31. McMillin, D. W., Negri, J. M., and Mitsiades, C. S. (2013). The role of tumour-stromal interactions in modifying drug response: challenges and opportunities. Nat Rev Drug Discov 12, 217-228.
32. Mielgo, A., and Schmid, M. C. (2013). Impact of tumour associated macrophages in pancreatic cancer. BMB reports 46, 131-138.
33. Mitchem, J. B., Brennan, D. J., Knolhoff, B. L., Belt, B. A., Zhu, Y., Sanford, D. E., Belaygorod, L., Carpenter, D., Collins, L., Piwnica-Worms, D., et al. (2013). Targeting tumor-infiltrating macrophages decreases tumor-initiating cells, relieves immunosuppression, and improves chemotherapeutic responses. Cancer Res 73, 1128-1141.
34. Murray, P. J., and Wynn, T. A. (2011). Protective and pathogenic functions of macrophage subsets. Nat Rev Immunol 11, 723-737.
35. Nakasone, Y., Fujimoto, M., Matsushita, T., Hamaguchi, Y., Huu, D. L., Yanaba, M., Sato, S., Takehara, K., and Hasegawa, M. (2012). Host-derived MCP-1 and MIP-1alpha regulate protective anti-tumor immunity to localized and metastatic B16 melanoma. Am J Pathol 180, 365-374.
36. Noy, R., and Pollard, J. W. (2014). Tumor-associated macrophages: from mechanisms to therapy. Immunity 41, 49-61.
37. Obenauf, A. C., Zou, Y., Ji, A. L., Vanharanta, S., Shu, W., Shi, H., Kong, X., Bosenberg, M. C., Wiesner, T., Rosen, N., et al. (2015). Therapy-induced tumour secretomes promote resistance and tumour progression. Nature.
38. Paulson, A. S., Tran Cao, H. S., Tempero, M. A., and Lowy, A. M. (2013). Therapeutic advances in pancreatic cancer. Gastroenterology 144, 1316-1326.
39. Pollak, M. (2012). The insulin and insulin-like growth factor receptor family in neoplasia: an update. Nat Rev Cancer 12, 159-169.
40. Pyonteck, S. M., Akkari, L., Schuhmacher, A. J., Bowman, R. L., Sevenich, L., Quail, D. F., Olson, O. C., Quick, M. L., Huse, J. T., Teijeiro, V., et al. (2013). CSF-1R inhibition alters macrophage polarization and blocks glioma progression. Nat Med 19, 1264-1272.
41. Qian, B. Z., and Pollard, J. W. (2010). Macrophage diversity enhances tumor progression and metastasis. Cell 141, 39-51.
42. Quail, D. F., and Joyce, J. A. (2013). Microenvironmental regulation of tumor progression and metastasis. Nat Med 19, 1423-1437.
43. Ruffell, B., Affara, N. I., and Coussens, L. M. (2012). Differential macrophage programming in the tumor microenvironment. Trends Immunol 33, 119-126.
44. Ruffell, B., and Coussens, L. M. (2015). Macrophages and Therapeutic Resistance in Cancer. Cancer Cell.
45. Schmid, M. C., Avraamides, C. J., Dippold, H. C., Franco, I., Foubert, P., Ellies, L. G., Acevedo, L. M., Manglicmot, J. R., Song, X., Wrasidlo, W., et al. (2011a). Receptor tyrosine kinases and TLR/IL1Rs unexpectedly activate myeloid cell PI3kgamma, a single convergent point promoting tumor inflammation and progression. Cancer Cell 19, 715-727.
46. Schmid, M. C., Avraamides, C. J., Foubert, P., Shaked, Y., Kang, S. W., Kerbel, R. S., and Varner, J. A. (2011b). Combined blockade of integrin-alpha4betal plus cytokines SDF-1alpha or IL-1beta potently inhibits tumor inflammation and growth. Cancer Res 71, 6965-6975.
47. Schmid, M. C., Franco, I., Kang, S. W., Hirsch, E., Quilliam, L. A., and Varner, J. A. (2013). PI3-kinase gamma promotes Rap1a-mediated activation of myeloid cell integrin alpha4beta1, leading to tumor inflammation and growth. PLoS One 8, e60226.
48. Schmittgen, T. D., and Livak, K. J. (2008). Analyzing real-time PCR data by the comparative C(T) method. Nat Protoc 3, 1101-1108.
49. Shree, T., Olson, 0. C., Elie, B. T., Kester, J. C., Garfall, A. L., Simpson, K., Bell-McGuinn, K. M., Zabor, E. C., Brogi, E., and Joyce, J. A. (2011). Macrophages and cathepsin proteases blunt chemotherapeutic response in breast cancer. Genes Dev 25, 2465-2479.
50. Sica, A., and Mantovani, A. (2012). Macrophage plasticity and polarization: in vivo veritas. J Clin Invest 122, 787-795.
51. Torres, M. P., Rachagani, S., Souchek, J. J., Mallya, K., Johansson, S. L., and Batra, S. K. (2013). Novel pancreatic cancer cell lines derived from genetically engineered mouse models of spontaneous pancreatic adenocarcinoma: applications in diagnosis and therapy. PLoS One 8, e80580.
52. Weizman, N., Krelin, Y., Shabtay-Orbach, A., Amit, M., Binenbaum, Y., Wong, R. J., and Gil, Z. (2014). Macrophages mediate gemcitabine resistance of pancreatic adenocarcinoma by upregulating cytidine deaminase. Oncogene 33, 3812-3819.
53. Werner, J., Combs, S. E., Springfeld, C., Hartwig, W., Hackert, T., and Buchler, M. W. (2013). Advanced-stage pancreatic cancer: therapy options. Nat Rev Clin Oncol 10, 323-333.
54. Wynn, T. A., Chawla, A., and Pollard, J. W. (2013). Macrophage biology in development, homeostasis and disease. Nature 496, 445-455.
55. Zhu, Y., Knolhoff, B. L., Meyer, M. A., Nywening, T. M., West, B. L., Luo, J., Wang-Gillam, A., Goedegebuure, S. P., Linehan, D. C., and DeNardo, D. G. (2014). CSF1/CSF1R blockade reprograms tumor-infiltrating macrophages and improves response to T-cell checkpoint immunotherapy in pancreatic cancer models. Cancer Res 74, 5057-5069.

1. A pharmaceutical composition comprising a first composition comprising an IGF inhibitor and a pharmaceutically acceptable excipient, adjuvant, diluent or carrier, and a second composition comprising a chemotherapeutic agent and a pharmaceutically-acceptable excipient, adjuvant, diluent or carrier, wherein the IGF inhibitor directly inhibits the biological activity of IGF.

2. The pharmaceutical composition according to claim 1, wherein the first and second compositions are provided in a form which is suitable for sequential, separate and/or simultaneous administration.

3. The pharmaceutical composition according to claim 1, further comprising a PDL-1 and/or CD80 inhibitor.

4. A method of treating a proliferative disorder in a subject, comprising administration to the subject of:
i) the pharmaceutical composition according to claim 1; or
ii) a combination comprising an IGF inhibitor and a chemotherapeutic agent, wherein the IGF inhibitor directly inhibits the biological activity of IGF.

5-6. (canceled)

7. The method according to claim 4, wherein the combination comprising an IGF inhibitor and a chemotherapeutic agent further comprises a PDL-1 inhibitor and/or a CD80 inhibitor.

8-11. (canceled)

12. The method according to, wherein the proliferative disorder is cancer.

13. The method according to claim 6, wherein the cancer is selected from pancreatic cancer, lung cancer, breast cancer, melanoma, colorectal cancer, ovarian cancer, gastric cancer, thyroid cancer, liver cancer, and prostate cancer, preferably wherein the proliferative disorder is pancreatic cancer.

14. The method according to claim 4, wherein a tumor sample is isolated from the patient, and wherein the tumor sample has increased levels of M2-like macrophages compared to a control sample or compared to a predetermined reference level.

15. (canceled)

16. The method according to claim 4, wherein said subject is susceptible to developing IGF-induced resistance to said chemotherapeutic agent.

17. The pharmaceutical composition according to claim 1, wherein the IGF inhibitor:
i) inhibits at least one IGF selected from IGF-1 and IGF-2;
ii) inhibits binding of at least one IGF to the insulin receptor, IGFR or hybrid receptors;
iii) is an anti-IGF antibody or an antigen binding fragment thereof; or
iv) any combination thereof.

18-20. (canceled)

21. The pharmaceutical composition according to claim 1, wherein the chemotherapeutic agent is selected from the group consisting of: a nucleoside analogue, a topoisomerase inhibitor, a platinum complex, and combinations thereof, optionally wherein the nucleoside analogue is gemcitabine or fluorouracil.

22-23. (canceled)

24. The pharmaceutical composition according to claim 3, wherein the PDL-1 inhibitor inhibits binding of PDL-1 to a PD-1 receptor; wherein the PDL-1 inhibitor is an anti-PDL-1 antibody or an antigen binding fragment thereof; or wherein the PDL-1 inhibitor is an anti-PDL-1 antibody or an antigen binding fragment thereof which inhibits binding of PDL-1 to a PD-1 receptor.

25. (canceled)

26. The pharmaceutical composition according to claim 3, wherein the CD80 inhibitor inhibits binding of CD80 to a CTLA-4 receptor; wherein the CD80 inhibitor is an anti-CD80 antibody or an antigen binding fragment thereof; or wherein the CD80 inhibitor is an anti-CD80 antibody or an antigen binding fragment thereof which inhibits binding of CD80 to a CTLA-4 receptor.

27-29. (canceled)

30. A method of increasing the sensitivity rate (efficacy rate) of a combination of an IGF inhibitor and a chemotherapeutic agent to treat cancer in a patient population, said method comprising selecting a sub population having a M2-like macrophage biomarker.

31-32. (canceled)e

33. A method of treating a subject having cancer comprising:
a. determining the level of M2-like macrophages in a tumor sample isolated from the subject;
b. comparing the level of M2-like macrophages in the tumor sample with the level of M2-like macrophages in a control sample or with a predetermined reference level for M2-like macrophages; and
c. administering a therapeutically effective amount of a chemotherapeutic agent and an IGF inhibitor when there is an increased level of M2-like macrophages in the tumor sample compared to the control sample or compared to the predetermined reference level.

34. The method according to claim 15, wherein the M2-like macrophage is CD163+.

35. The method according to claim 15, wherein the predetermined reference level of M2-like macrophages is at least 20 M2-like macrophages in the tumour core sample.

36. The method according to claim 15, wherein the cancer is pancreatic cancer or breast cancer.

37. The method according to claim 15, wherein the chemotherapeutic agent is gemcitabine or fluorouracil.

38-45. (canceled)
